(19) United States
(12) Patent Application Publication
(10) Pub. No.: US 2010/0029740 A1
(43) Pub. Date: Feb. 4, 2010

(54) AZABICYCLIC COMPOUNDS AS SEROTONIN, DOPAMINE AND NOREPINEPHRINE RE-UPTAKE INHIBITORS

(76) Inventors: Giorgio Bonanomi, Verona (IT); Fabrizio Micheli, Verona (IT); Silvia Terreni, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/519,438

(22) PCT Filed: Dec. 12, 2007
(86) PCT No.: PCT/EP2007/063841

§ 371 Date: Jun. 16, 2009

(30) Foreign Application Priority Data
Dec. 18, 2006 (GB) 0625198.7

(51) Int. Cl. A61K 31/403; C07D 209/52; A61P 25/00

(52) U.S. Cl. 514/412; 548/515

(57) ABSTRACT

Novel compounds of formula (I)′ and pharmaceutically acceptable salts, solvates and prodrugs thereof:

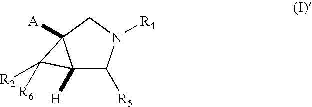

wherein
A is:

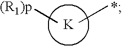

K is a mono or bicyclic aryl group;
R₁ is selected from halogen, C₁₋₄alkyl and C₁₋₄alkoxy (R₁ may assume different meanings on the basis of p value);
p is an integer from 0 to 5;
R₂ is a group P:

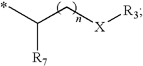

R₃ is hydrogen, C₁₋₄alkyl, C₃-₆cycloalkyl, C₃-₆cycloalkylC₁₋₃alkyl, haloC₁₋₂alkyl or an optionally substituted phenyl group;
X is oxygen, —NR₈— or sulphur;
n is 0 or 1;
R₇ is hydrogen or methyl;
R₈ is hydrogen or C₁₋₄alkyl;
R₄ is hydrogen or methyl;
R₅ is hydrogen or C₁₋₄alkyl; and
R₆ is hydrogen or C₁₋₄alkyl;
   processes for their preparation, intermediates used in these processes, pharmaceutical compositions containing them and their use in therapy, as serotonin (5-HT), dopamine (DA) and norepinephrine (NE), re-uptake inhibitors.

The present invention relates to novel compounds, processes for their preparation, intermediates used in these processes, pharmaceutical compositions containing them and their use in therapy, as serotonin (5-HT), dopamine (DA) and norepinephrine (NE), re-uptake inhibitors.

Brain tissue is constituted of neuronal cells which are able to communicate with each other thanks to specific cellular structures named synapses. The exchange of signals between neurons in the synapses happens through neurochemical messengers named neurotransmitters, acting on specific target protein molecules, both post and pre-synaptic, being referred to as receptors. Monoamines represent a family of small neurotransmitter molecules sharing common chemical features, and include serotonin (5-HT), dopamine (DA) and norepinephrine (NE).

Monoamine neurotransmitters are released into the synaptic cleft between neurons and interact with receptors present on the membrane of the target cells. The switch of the neurochemical signal occurs mainly by removal of the neurotransmitter molecules through other protein molecules referred to as monoamine transporters (SERT for 5-HT, DAT for DA and NET for NE). Transporters are able to bind neurotransmitter molecules and moving them into the presynaptic terminals, being this cellular mechanism referred to as re-uptake. The pharmacological inhibition of the re-uptake process can cause an increase of monoamine at synaptic level and as a consequence an enhancement of the physiological activity of neurotransmitter tone.

The serotonergic neurotransmission in the brain is mediated by a large family of receptors belonging to both the G-protein coupled receptors and ligand-gated ion channels including 14 subtypes, and it is involved in a vast variety of physiologic functions. Compounds endowed of inhibitory properties at the SERT are predicted to have the ability to treat in mammals, including humans, a variety of disorders associated with this neural system, for example eating disorders, major depression and mood disorders, obsessive compulsive disorders, panic disorders, alcoholism, pain, memory deficits and anxiety. Included among these disorders are disorders related to depression, such as pseudodementia or Ganser's syndrome, migraine pain, bulimia, obesity, pre-menstrual syndrome or late luteal phase syndrome, tobacco abuse, panic disorder, post-traumatic syndrome, memory loss, dementia of ageing, acquired immunodeficiency syndrome dementia complex, memory dysfunction in ageing, social phobia, attention deficit hyperactivity disorder, chronic fatigue syndrome, premature ejaculation, erectile difficulty, anorexia nervosa, disorders of sleep, autism, mutism or trichotillomania.

Major depression is an affective disorder, or disorder of mood, characterized by several symptoms including feeling of profound sadness, worthlessness, despair and loss of interest in all pleasures (anhedonia), recurrent thoughts of death, mental slowing, loss of energy an inability to take decision, often associated with anxiety and agitation. These symptoms are persistent and can range from mild to severe.

The pathophysiology of major depression is poorly understood being a multifactorial syndrome and, due to this, several neurotransmitter systems have been implicated. However, it is generally believed that the disorder stems from a decrease in the synaptic concentration of monoamine neurotransmitters, mainly NE and 5-HT, in critical brain areas, leading to the “monoamine theory” of depression.

Several lines of preclinical and clinical evidence indicate that an enhancement of serotonin-mediated neurotransmission might be effective in the treatment of major depression and actually the selective serotonin re-uptake inhibitors (SSRIs) have come to dominate the therapy of depression over the last two decades. Fluoxetine, the first SSRI to be introduced, is the prototype of this group. Other members include Paroxetine, Sertraline, Fluvoxamine, Citalopram.

However, it is not clear exactly how these agents act to relieve depression. As with other classes of antidepressant, there is a lag of several weeks before the onset of the mood-elevating effect, despite the rapid blockade of the serotonin re-uptake. It is presumed that secondary adaptive changes must occur at serotonergic synapses after chronic administration of SSRIs i.e. down-regulation of release-regulating autoreceptors and increased neurotransmitter release. The delayed onset of anti-depressant effect is considered to be a serious drawback to currently used SSRIs. Moreover, although a generally good tolerability of SSRIs, the elevation of 5-HT levels at central and peripheral synapses leas to stimulation of receptor subtypes like 5-HT_{2C} and 5-HT₃, which contributes to agitation and restless, along with gastrointestinal and sexual side-effects.

The success of the SSRIs rekindled interest in the development of selective norepinephrine re-uptake inhibitors (SNRIs) as potential antidepressant. A number of such compounds have been synthesized, e.g. Nisoxetine, Maprotiline, Tomoxetine and Reboxetine. Furthermore, many compounds, including old tricyclic antidepressant, have a mixed NET and SERT inhibition profile, like Imipramine and Amitriptyline (with SERT potency>NET) and Desipramine, Nortriptyline, and Protriptyline (NET>SERT blockade).

The pharmacological manipulation of the DAT can in principle have the ability to elevate DA levels in the mesolimbic system, reversing the anhedonia that is a core symptom of major depression. A DAT inhibition component, in combination to a blockade of SERT and NET, can also have the ability to improve the lack of motivation and attention and enhance cognitive deficits seen in depressed patients. On the other hand, blockade of DAT has to be carefully managed in order to avoid potential reinforcing effects and abuse liability. However compounds with DAT inhibition in their pharmacology, such as Dexmethylphenidate, Methylphenidate and Bupropion, have been successfully marketed. Clinical studies indicate that patients with poor response to SSRIs benefit from combination therapy with agents that enhance dopaminergic tone. As a result, compounds with a strong SERT inhibiting activity combined with a well balanced NET blockade and moderate DAT inhibiting activity may therefore provide a replace for current combination therapies for treating unresponsive patients, providing greater efficacy and therapeutic flexibility with a more rapid onset of anti-depressant effect.

Due to their valuable DAT inhibition, the compounds of the present invention are considered useful for the treatment of Parkinsonism, depression, obesity, narcolepsy, drug addiction or misuse, including cocaine abuse, attention-deficit hyperactivity disorders, Gilles de la Tourettes disease and senile dementia. Dopamine re-uptake inhibitors enhances indirectly via the dopamine neurones the release of acetylcholin and are therefore also useful for the treatment of memory deficits, e.g. in Alzheimers disease, presenile dementia, memory dysfunction in ageing, and chronic fatigue syndrome. Noradrenaline re-uptake inhibitors are considered useful for enhancing attention, alertness, arousal, vigilance and for treating depression.

The object of the present invention is to provide novel compounds which are serotonin (5-HT), dopamine (DA) and norepinephrine (NE), re-uptake inhibitors.

In a first aspect, the present invention provides a compound of formula (I) or a pharmaceutically acceptable salt, solvate or prodrug thereof:

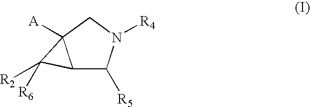

wherein
A is

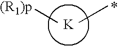

and
K is a mono or bicyclic aryl group;
R₁ is selected from a group consisting of: halogen, C₁₋₄alkyl and C₁₋₄alkoxy, and such R₁ may assume different meanings on the basis of p value;
p is an integer from 0 to 5;
R₂ is a group P
wherein P is

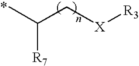

and R₃ is hydrogen, C₁₋₄alkyl, C₃-₆cycloalkyl, C₃-₆cycloalkylC₁₋₃alkyl, haloC₁₋₂alkyl or an optionally substituted phenyl group;
   X is oxygen, —NR₈— or sulphur;
   n is 0 or 1;
   R₇ is hydrogen or methyl;
R₄ is hydrogen or methyl;
R₅ is hydrogen or C₁₋₄alkyl;
R₆ is hydrogen or C₁₋₄alkyl;
and R₈ is hydrogen or C₁₋₄alkyl.

In another embodiment, the present invention provides a compound of formula (IA) or a pharmaceutically acceptable salt, solvate or prodrug thereof:

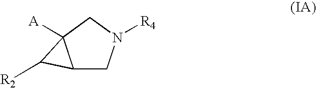

wherein
A is

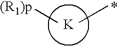

and
K is a mono or bicyclic aryl group;
R₁ is selected from a group consisting of: halogen, C₁₋₄alkyl and C₁₋₄alkoxy, and such R₁ may assume different meanings on the basis of p value;
p is an integer from 0 to 5;
R₂ is a group P or P₁
wherein
P is

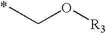

P₁ is

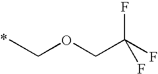

R₃ is hydrogen, C₁₋₄alkyl, C₃-₆cycloalkyl or C₃-₆cycloalkylC₁₋₃alkyl;
and R₄ is hydrogen or methyl.

The term ‘C₃-C₆ cycloalkyl group’ as used herein means a non aromatic monocyclic hydrocarbon ring of 3 to 6 carbon atom such as, for example, cyclopropyl, cyclobutyl, cyclopentyl, or cyclohexyl; while unsaturated cycloalkyls include cyclopentenyl and cyclohexenyl, and the like.

The term ‘C₃-₆cycloalkylC₁₋₃alkyl’ as used herein means an alkyl having from one to three carbon atoms and wherein one hydrogen atom is replaced with a C₃-C₆ cycloalkyl group as above defined, for example methylcyclopropane.

The term “C₁₋₄alkoxy” refers to a straight chain or branched chain alkoxy (or “alkyloxy”) group having from one to four carbon atoms, such as methoxy, ethoxy, propoxy, isopropoxy, butoxy, isobutoxy, sec-butoxy and tert-butoxy.

The term “halogen” and its abbreviation “halo” refer to fluorine (F), chlorine (Cl), bromine (Br) or iodine (I). Where the term “halo” is used before another group, it indicates that the group is substituted by one, two or three halogen atoms

The term ‘aryl’ as used herein means an aromatic carbocyclic moiety; such as phenyl, if monocyclic moiety, biphenyl or naphtyl, if bicyclic moiety.

The term ‘halo C₁₋₂ alkyl group’ as used herein may be a C₁₋₂ alkyl group as defined before substituted with at least one halogen, preferably fluorine, such as —CH₂CF₃, —CHF₂, or —CF₃.

Any of these groups may be attached to the rest of the molecule at any suitable position.

With regard to stereoisomers, the compounds of structure (I) may have at least three or more asymmetric carbon atoms and may occur as racemates, racemic mixtures, enantiomers and as individual diastereoisomers. All such isomeric forms are included within the present invention, including mixtures thereof.

When a specific enantiomer or diastereoisomer of a compound of formula (I) or salts thereof, is required, this may be obtained for example by resolution of a corresponding enantiomeric or diastereoisomeric mixture using conventional methods.

Thus, for example, specific enantiomers or diastereoisomers of the compounds may be obtained from the corresponding enantiomeric or diastereoisomeric mixture using chiral chromatographic methods such as for example chiral HPLC.

Furthermore a specific stereoisomer, enantiomer or diastereoisomer, of a compound of the invention may be synthesized from the appropriate optically active intermediate using any of the general processes described herein.

Optically active intermediates or stereochemically enriched intermediates, may be generated by resolution of a corresponding enantiomeric or diastereoisomeric mixtures using conventional methods, or by performance of stereoselective reactions or by combining different resolution techniques.

Also specific enantiomers or diastereoisomers of the compounds may be obtained by combining conventional methods above described.

The absolute configuration of the optical isomers of some compounds of the present invention was assigned using ab initio VCD (vibrational circular dichroism).

Chiral molecules exhibit vibrational circular dichroism (VCD). Vibrational circular dichroism (VCD) is the differential interaction of a chiral molecule with left and right circularly polarized infrared radiation during vibrational excitation.

The VCD spectrum of a chiral molecule is dependent on its three-dimensional structure. Most importantly, the VCD spectrum of a chiral molecule is a sensitive function of its absolute configuration and, in the case of flexible molecules, of its conformation. In principle, therefore, VCD permits the determination of the structure of a chiral molecule. VCD spectra were first measured in the 1970s. Subsequently, VCD instrumentation has developed enormously in spectral range and in sensitivity. Currently, VCD spectra of liquids and solutions can be measured over the majority of the fundamental infrared (IR) spectral range (v≧650 cm-1) with high sensitivity at acceptable resolution (1-5 cm-1) using both dispersive and Fourier Transform (FT) VCD instrumentation. Very recently, commercial FT VCD instrumentation has become available, greatly enhancing the accessibility of VCD spectra.

The use of VCD as a reliable method for the determination of absolute configuration of chiral molecules is now well established (see for example Shah R D, et al., Curr Opin Drug Disc Dev 2001;4:764-774; Freedman T B, et al., Helv Chim Acta 2002; 85:1160-1165; Dyatkin A B, et al. Chirality 2002;14:215-219; Solladie-Cavallo A, Balaz Met al., Tetrahedron Assym 2001;12:2605-2611; Nafie L A, et al. Circular dichroism, principles and applications, 2nd ed. New York: John Wiley & Sons; 2000. p 97-131; Nafie L A, et al. in: Yan B, Gremlish H-U, editors. Infrared and Raman spectroscopy of biological materials. New York: Marcel Dekker; 2001. p 15-54; Polavarapu P L, et al., J Anal Chem 2000;366:727-734; Stephens P J, et al., Chirality 2000;12:172-179; Solladie-Cavallo A, et al., Eur J Org Chem 2002: 1788-1796).

The method entails comparison of observed IR and VCD spectra with calculations of the spectra for a specific configuration and provides information both on the absolute configuration and on the solution conformation.

Given an experimental spectrum of a chiral molecule whose absolute configuration and/or conformation are unknown and to be determined, the general procedure is as follows: 1) all possible structures are defined; 2) the spectra of these structures are predicted; and 3) predicted spectra are compared to the experimental spectrum. The correct structure will give a spectrum in agreement with experiment; incorrect structures will give spectra in disagreement with experiment.

VCD spectra are always measured simultaneously with vibrational unpolarized absorption spectra (“infrared (IR) spectra”) and the two vibrational spectra together provide more information than does the VCD spectrum alone. In addition, vibrational unpolarized absorption spectra are automatically predicted simultaneously with VCD spectra.

For ab initio assignments, VCD and unpolarized IR spectra were calculated using the Gaussian 98 software package.

It will be appreciated by a person skilled in the art that compounds of formula (I) possess at least three chiral centres, namely at position 1, 5 and 6 in the 1-azabicyclo[3.1.0]hexane portion of the molecule. Because of the presence of the fused cyclopropane, compounds of formula (I) are believed to have a “cis” disposition of the substituents (both groups linked to the bicyclic ring system are on the same face of this bicyclic ring system) as shown for compounds of formula (I)′.

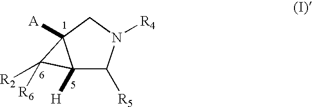

Thus, in one embodiment of the present invention compounds of formula (I)′ are provided, or pharmaceutically acceptable salts, solvates or prodrugs thereof, having “cis” disposition, represented by the bold highlight of the two bonds near the cyclopropyl moiety:

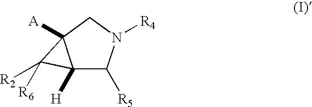

wherein R₄, R₂, R₅, R₆ and A, are defined as above for compounds of formula (I).

It will be appreciated by a person skilled in the art, that compounds of formula (I)′ may have relative exo or endo stereochemistry generated by the relative disposition in the space of the group R₂ with respect to the group A and the hydrogen atom on the cis junction.

The structures below show the relative exo/endo stereochemistry for compounds of formula endo-(I)′ and exo-(I)′:

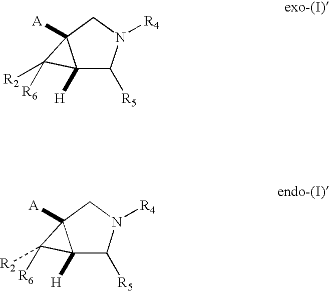

The bold highlight of the bonds in compound of formula exo-(I)′ indicates that the group R₂, the group A and the hydrogen on the cis junction are located on the same face of the cyclopropane ring.

The bold/dotted highlight of the bonds in compound of formula endo-(I)′ indicates that the group R₂, the group A and the hydrogen on the cis junction are located on the opposite face of the cyclopropane ring.

Thus, it will be understood by the person skilled in the art that the compounds of formula (I)′ may exist in at least two couple of stereoisomers of formula (IB) and (IC), namely enantiomers at position 1 and 5 of the bicyclic ring, as shown below:

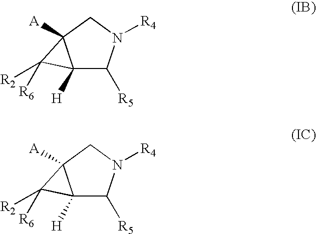

In one embodiment of the present invention, in compounds of formula (I)′ the bold highlight of the two bonds near the cyclopropyl moiety bearing group A and H indicate, mixtures (including but not limited to racemic mixtures) of cis isomers (IB) and (IC).

In another embodiment of the present invention, in compounds of formula (I)′ the bold highlight of the two bonds near the cyclopropyl moiety bearing group A and H, indicate a cis stereoisomer of formula (IB) or (IC) enriched in a single absolute configuration at stereogenic centers named 1 and 5.

It is intended in the context of the present invention that stereochemical isomers of formula (IB) or (IC) are enriched in one configuration at centers named 1 and 5. In one embodiment, the isomers correspond to at least 90% e.e. (enantiomeric excess). In another embodiment the isomers correspond to at least 95% e.e. In another embodiment the isomers correspond to at least 99% e.e.

It will also be appreciated by a person skilled in the art that the compounds of formula (I)′ may exist at least in four stereoisomers of formula (ID), (IE) [exo stereochemistry, generated by the relative disposition in the space of the group R₂ with respect to the group A and the hydrogen atom on the cis junction], (IF) and (IG) [endo stereochemistry, generated by the relative disposition in the space of the group R₂ with respect to the group A and the hydrogen atom on the cis junction], as shown below:

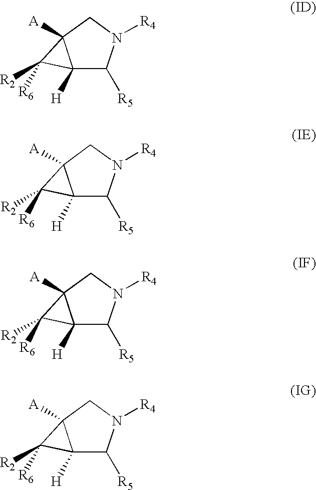

In one embodiment of the present invention, the bold highlight of the bonds in compounds of formula exo-(1)′ is intended to represent mixtures of stereoisomers of formula (ID) and (IE).

In one embodiment of the present invention, the bold highlight of the bonds in compounds of formula exo-(I)′ is intended to represent a stereoisomer of formula (ID) or (IE) enriched in a single absolute configuration at stereogenic centers named 1,5 and 6.

In one embodiment of the present invention, the bold/dotted highlight of the bonds in compounds of formula endo-(I)′ is intended to represent mixtures of stereoisomers of formula (IF) and (IG).

In one embodiment of the present invention, the bold/dotted highlight of the bonds in compounds of formula endo-(I)′ is intended to represent a stereoisomer of formula (IF) or (IG) enriched in a single absolute configuration at stereogenic centers named 1,5 and 6.

It is intended in the context of the present invention that stereochemical isomers of formula (ID), (IE), (IF) and (IG) are enriched in one configuration at centers named 1, 5 and 6. In one embodiment, the isomers correspond to at least 90% e.e. (enantiomeric excess). In another embodiment the isomers correspond to at least 95% e.e. In another embodiment the isomers correspond to at least 99% e.e.

It will be appreciated by the person skilled in the art that in compounds of formula endo-(I)′ and exo-(1)′, when R₅ is not hydrogen, this substituent may adopt syn or anti configuration with respect to group A, leading to an increased number of stereoisomers.

The structures below show the relative exo/endo stereochemistry for compounds of formula endo-(IL), endo-(IM), exo-(IN) and exo-(IO), wherein R₅ is not hydrogen:

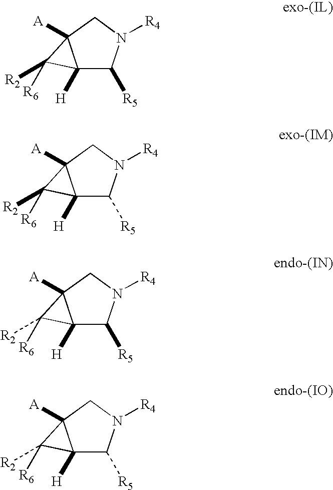

The bold highlight of the bonds in compounds of formula exo-(IL) indicates that the group R₂, the group A and the hydrogen on the cis junction are located on the same face of the cyclopropane ring and that the group R₅ and the group A are on the same face of the pyrrolidine ring.

The bold and dotted highlight of the bonds in compounds of formula endo-(IN) indicates that the group R₂ is located on the opposite face of the cyclopropane ring with respect to the group A and the hydrogen on the cis junction and that the group R₅ and the group A are on the same face of the pyrrolidine ring.

The bold and dotted highlight of the bonds in compounds of formula exo-(IM) indicates that the group R₂, the group A and the hydrogen on the cis junction are located on the same face of the cyclopropane ring and that the group R₅ and the group A are on the opposite face of the pyrrolidine ring.

The bold and dotted highlight of the bonds in compounds of formula endo-(IO) indicates that the group R₂ is located on the opposite face of the cyclopropane ring with respect to the group A and the hydrogen on the cis junction and that the group R₅ and the group A are on the opposite face of the pyrrolidine ring.

It will also be appreciated by a person skilled in the art that the compounds of formula exo-(I)′, when R₅ is not hydrogen, may exist at least in four stereoisomers of formula (IP), (IQ), (IR) and (IS) as shown below:

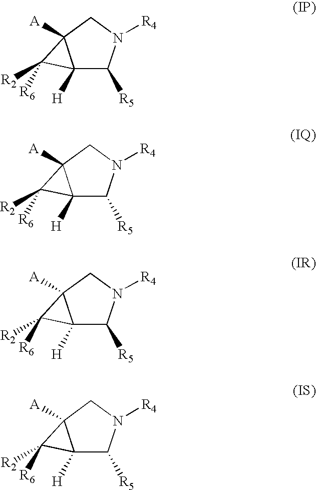

In one embodiment of the present invention, the bold highlight of the bonds in compounds of formula exo-(IL) or exo-(IM) is intended to represent, respectively, mixtures of stereoisomers of formula (IP) and (IS) or mixtures of stereoisomers of formula (IQ) and (IR).

All features and embodiments of compounds of formula (I) apply to compounds of formula (I)′, (IA), (IB), (ID), (IE), (IF), (IG), (IH), (IL), (IM), (IN), (IO), (IP), (IQ), (IR), (IS) mutatis mutandis.

It will also be appreciated, in common with most biologically active molecules that the level of biological activity may vary between the individual stereoisomers of a given molecule. It is intended that the scope of the invention includes all individual stereoisomers (diastereoisomers and enantiomers) and all mixtures thereof, including but not limited to racemic mixtures, which demonstrate appropriate biological activity with reference to the procedures described herein.

In one embodiment, K is a phenyl group or a naphtyl group. In another embodiment, K is a phenyl group. In a further embodiment K is a naphtyl group.

In one embodiment, R, is halogen. In another embodiment R₁ is chloro.

In one embodiment, p is 0, 1 or 2. In another embodiment p is 0 or 2. In a further embodiment p is 0. In a still further embodiment, p is 2.

In one embodiment, n is 0 or 1. In another embodiment, n is 0. In a further embodiment, n is 1.

In one embodiment, X is oxygen, —NR₈— or sulphur. In another embodiment, X is oxygen. In a further embodiment, X is —NR₈— or sulphur. In a still further embodiment, X is sulphur.

In one embodiment, R₃ is hydrogen, C₁₋₄alkyl, C₃-₆cycloalkyl, C₃-₆cycloalkylC₁₋₃alkyl, haloC₁₋₂alkyl or an optionally substituted phenyl group. In another embodiment, R₃ is hydrogen. In a further embodiment, R₃ is C₁₋₄alkyl. In a still further embodiment, R₃ is C₃-₆cycloalkyl, C₃-₆cycloalkylC₁₋₃alkyl, haloC₁₋₂alkyl or an optionally substituted phenyl group. In an additional embodiment, R₃ is C₃-₆cycloalkyl, C₃-₆cycloalkylC₁₋₃alkyl or haloC₁₋₂alkyl.

In one embodiment, R₄ is hydrogen or methyl. In another embodiment R₄ is hydrogen.

In one embodiment, R₆ is hydrogen or methyl. In another embodiment R₆ is hydrogen.

In one embodiment, R₅ is hydrogen or methyl. In another embodiment R₅ is hydrogen.

In one embodiment, R₇ is hydrogen or methyl. In another embodiment R₇ is hydrogen.

In one embodiment, R₈ is hydrogen or methyl.

In one embodiment, K is a naphtyl group and p is 0.

In another embodiment, K is a phenyl group, p is 2 and R₁ is chloro.

In another embodiment of the present invention compounds of formula (IH), salts, solvates or prodrugs thereof are provided, which correspond to the compounds of formula (I) having “cis” disposition, represented by the bold highlight of the two bonds near the cyclopropyl moiety:

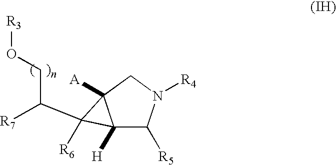

wherein R₃, R₄, R₆, R₇, R₅, n and A, are defined as above for compounds of formula (I).

In Formula (IH), in one embodiment, R₄ is hydrogen or methyl, R₅ is hydrogen, R₇ is hydrogen or methyl, n is 0 or 1, R₆ is hydrogen or methyl and A is 3,4-dichlorohenyl or a naphtyl group.

In Formula (IH), in another embodiment, R₄is hydrogen, R₅is hydrogen, R₇ is hydrogen, n is 0, R₆ is hydrogen and A is 3,4-dichlorohenyl or a naphtyl group.

In Formula (IH), in a further embodiment, R₄ is hydrogen, R₅ is hydrogen, R₇ is hydrogen, n is 0, R₆ is hydrogen, R₃ is C₁₋₄alkyl and A is 3,4-dichlorohenyl.

Certain groups/substituents included in the present invention may be present as isomers. The present invention includes within its scope all such isomers, including racemates, enantiomers, tautomers and mixtures thereof.

Certain groups in compounds of formula (I) or in intermediates used to prepare them may exist in one or more tautomeric forms. The present invention includes within its scope all such tautomeric forms, including mixtures.

As used herein, the term “salt” refers to any salt of a compound according to the present invention prepared from an inorganic or organic acid or base, quaternary ammonium salts and internally formed salts and also includes pharmaceutically acceptable salts. Pharmaceutically acceptable salts are particularly suitable for medical applications because of their greater aqueous solubility relative to the parent compounds. Such salts must clearly have a physiologically acceptable anion or cation.

Salts of compounds of formula (I) may be prepared through conventional methods and are included within the scope of the present invention.

Certain of the compounds of the invention may form acid or base addition salts with one or more equivalents of the acid or of the base. The present invention includes within its scope all possible stoichiometric and non-stoichiometric forms.

Pharmaceutically acceptable salts may also be prepared from other salts, including other pharmaceutically acceptable salts, of the compound of formula (I) using conventional methods.

Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, naphtoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; and internally formed salts. Salts having a non-pharmaceutically acceptable anion are within the scope of the invention as useful intermediates for the preparation of pharmaceutically acceptable salts and/or for use in non-therapeutic, for example, in vitro, situations.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The compounds of formula (I) may readily be isolated in association with solvent molecules by crystallization or evaporation of an appropriate solvent to give the corresponding solvates.

In addition, prodrugs are also included within the context of this invention. As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130.

Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I).

Hereinafter, compounds of formula (I) and their pharmaceutically acceptable salts, solvates and prodrugs defined in any aspect of the invention (except intermediate compounds in chemical processes) are referred to as “compounds of the invention”.

Furthermore, some of the crystalline forms of the compounds of the present invention, may exist as polymorphs, which are included in the present invention.

Those skilled in the art will appreciate that in the preparation of the compounds of the invention, it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl). Examples of suitable oxygen protecting groups may include for example alky silyl groups, such as trimethylsilyl or tert-butyldimethylsilyl; alkyl ethers such as tetrahydropyranyl or tert-butyl; or esters such as acetate.

The present invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, 35S, ¹⁸F, ³⁶Cl, ¹²³I and ¹²⁵I.

Compounds of the present invention and non-pharmaceutically acceptable salts thereof that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and non-pharmaceutically acceptable salts thereof can generally be prepared by carrying out the procedures disclosed in the Schemes and/or in the Examples below, by substituting a readily available isotopically labelled reagent for a non-isotopically labelled reagent.

Example compounds of the present invention include:

(1S,5S,6S/1R,5R,6R)-1-(3,4-dichlorophenyl)-3-azabicyclo[3.1.0]hex-6-yl]methanol;

(1S,5S,6R/1R,5R,6S)-1-(3,4-dichlorophenyl)-3-azabicyclo[3.1.0]hex-6-yl]methanol;

(1S,5S,6S/1R,5R,6R)-1-(3,4-dichlorophenyl)-6-[(methyloxy)methyl]-3-azabicyclo[3.1.0]hexane;

(1S,5S,6R/1R,5R,6S)-1-(3,4-dichlorophenyl)-6-[(methyloxy)methyl]-3-azabicyclo[3.1.0]hexane;

(1S,5S,6S/1R,5R,6R)-6-{[(cyclopropylmethyl)oxy]methyl}-1-(3,4-dichlorophenyl)-3-azabicyclo[3.1.0]hexane;

(1S,5S,6R/1R,5R,6S)-6-{[(cyclopropylmethyl)oxy]methyl}-1-(3,4-dichlorophenyl)-3-azabicyclo[3.1.0]hexane;

(1S,5S,6S/1R,5R,6R)-1-(3,4-dichlorophenyl)-6-[(ethyloxy)methyl]-3-azabicyclo[3.1.0]hexane;

(1S,5S,6S/1R,5R,6R)-1-(3,4-dichlorophenyl)-6-[(propyloxy)methyl]-3-azabicyclo[3.1.0]hexane;

(1S,5S,6S/1R,5R,6R)-1-(3,4-dichlorophenyl)-6-{[(2,2,2-trifluoroethyl)oxy]methyl}-3-azabicyclo[3.1.0]hexane;

[(1S,5S,6S/1R,5R,6R)]-6-[(methyloxy)methyl]-1-(2-naphthalenyl)-3-azabicyclo[3.1.0]hexane;

or a pharmaceutically acceptable salts thereof.

Example compounds of the present invention include:

[(1S,5S,6R/1R,5R,6S)-1-(3,4-dichlorophenyl)-3-azabicyclo[3.1.0]hex-6-yl]methanol;

(1S,5S,6S/1R,5R,6R)-1-(3,4-dichlorophenyl)-6-[(methyloxy)methyl]-3-azabicyclo[3.1.0]hexane;

(1S,5S,6S/1R,5R,6R)-1-(3,4-dichlorophenyl)-6-[(methyloxy)methyl]-3-azabicyclo[3.1.0]hexane;

(1S,5S,6R/1R,5R,6S)-1-(3,4-dichlorophenyl)-6-[(methyloxy)methyl]-3-azabicyclo[3.1.0]hexane;

(1S,5S,6S/1R,5R,6R)-6-{[(cyclopropylmethyl)oxy]methyl}-1-(3,4-dichlorophenyl)-3-azabicyclo[3.1.0]hexane;

(1S,5S,6S/1R,5R,6R)-6-{[(cyclopropylmethyl)oxy]methyl}-1-(3,4-dichlorophenyl)-3-azabicyclo[3.1.0]hexane;

(1S,5S,6R/1R,5R,6S)-6-{[(cyclopropylmethyl)oxy]methyl}-1-(3,4-dichlorophenyl)-3-azabicyclo[3.1.0]hexane;

(1S,5S,6S)-1-(3,4-dichlorophenyl)-6-[(ethyloxy)methyl]-3-azabicyclo[3.1.0]hexane;

(1R,5R,6R)-1-(3,4-dichlorophenyl)-6-[(ethyloxy)methyl]-3-azabicyclo[3.1.0]hexane;

(1S,5S,6S/1R,5R,6R)-1-(3,4-dichlorophenyl)-6-[(ethyloxy)methyl]-3-azabicyclo[3.1.0]hexane;

(1S,5S,6S/1R,5R,6R)-1-(3,4-dichlorophenyl)-6-[(propyloxy)methyl]-3-azabicyclo[3.1.0]hexane;

(1S,5S,6S/1R,5R,6R)-1-(3,4-dichlorophenyl)-6-{[(2,2,2-trifluoroethyl)oxy]methyl}-3-azabicyclo[3.1.0]hexane;

([(1S,5S,6S/1R,5R,6R)]-6-[(methyloxy)methyl]-1-(2-naphthalenyl)-3-azabicyclo[3.1.0]hexane;

(1S,5S,6S or 1R,5R,6R)-1-(3,4-dichlorophenyl)-6-[(ethyloxy)methyl]-3-methyl-3-azabicyclo[3.1.0]hexane;

(1R,5R,6R/1S,5S,6S)-1-(3,4-dichlorophenyl)-6-{[(1-methylethyl)oxy]methyl}-3-azabicyclo[3.1.0]hexane;

(1R,5R,6R or 1S,5S,6S)-1-(3,4-dichlorophenyl)-6-{[(1-methylethyl)oxy]methyl}-3-azabicyclo[3.1.0]hexane;

(1R,5R,6R or 1S,5S,6S)-1-(3,4-dichlorophenyl)-6-{[(1-methylethyl)oxy]methyl}-3-azabicyclo[3.1.0]hexane;

(1R,5R,6R/1S,5S,6S)-1-(3,4-dichlorophenyl)-6-{[(1-methylethyl)oxy]methyl}-3-azabicyclo[3.1.0]hexane;

(1R,5R,6R/1S,5S,6S)-6-[(cyclobutyloxy)methyl]-1-(3,4-dichlorophenyl)-3-azabicyclo[3.1.0]hexane;

(1R,5R,6R or 1S,5S,6S)-6-[(cyclobutyloxy)methyl]-1-(3,4-dichlorophenyl)-3-azabicyclo[3.1.0]hexane;

(1R,5R,6R or 1S,5S,6S)-6-[(cyclobutyloxy)methyl]-1-(3,4-dichlorophenyl)-3-azabicyclo[3.1.0]hexane;

(1R,5R,6R/1S,5S,6S)-6-[(cyclopentyloxy)methyl]-1-(3,4-dichlorophenyl)-3-azabicyclo[3.1.0]hexane;

(1R,5R,6R or 1S,5S,6S)-6-[(cyclopentyloxy)methyl]-1-(3,4-dichlorophenyl)-3-azabicyclo[3.1.0]hexane;

(1R,5R,6R or 1S,5S,6S)-6-[(cyclopentyloxy)methyl]-1-(3,4-dichlorophenyl)-3-azabicyclo[3.1.0]hexane;

(1R,5R,6R/1S,5S,6S)-6-[(cyclohexyloxy)methyl]-1-(3,4-dichlorophenyl)-3-azabicyclo[3.1.0]hexane trifluoroacetate;

(1S,5R,6R/1R,5S,6S)-1-(3,4-dichlorophenyl)-6-propyl-3-azabicyclo[3.1.0]hexane trifluoroacetate;

[(1S,2S,5S,6S/1R,2R,5R,6R)-1-(3,4-dichlorophenyl)-6-[(ethyloxy)methyl]-2-methyl-3-azabicyclo[3.1.0]hexane;

[(1S,2R,5S,6S/1R,2S,5R,6R)-1-(3,4-dichlorophenyl)-6-[(ethyloxy)methyl]-2-methyl-3-azabicyclo[3.1.0]hexane;

[(1S,2R,5S,6S or 1R,2S,5R,6R)-1-(3,4-dichlorophenyl)-6-[(ethyloxy)methyl]-2-methyl-3-azabicyclo[3.1.0]hexane;

[(1S,2R,5S,6S or 1R,2S,5R,6R)-1-(3,4-dichlorophenyl)-6-[(ethyloxy)methyl]-2-methyl-3-azabicyclo[3.1.0]hexane;

(1S,5S/1R,5R)-1-(3,4-dichlorophenyl)-6-[(1R/1S)-1-(methyloxy)ethyl]-3-azabicyclo[3.1.0]hexane;

(1R,5R,6R/1S,5S,6S) and (1R,5R,6S/1S,5S,6R)-1-(3,4-dichlorophenyl)-6-[(ethyloxy)methyl]-6-methyl-3-azabicyclo[3.1.0]hexane;

[(1R,5S,6S/1S,5R,6R)-1-(3,4-dichlorophenyl)-3-azabicyclo[3.1.0]hex-6-yl]ethanol;

(1R,5S,6S/1S,5R,6R)-1-(3,4-dichlorophenyl)-6-[2-(methyloxy)ethyl]-3-azabicyclo[3.1.0]hexane;

(1R,5S,6S or 1S,5R,6R)-1-(3,4-dichlorophenyl)-6-[2-(methyloxy)ethyl]-3-azabicyclo[3.1.0]hexane;

(1R,5S,6S or 1S,5R,6R)-1-(3,4-dichlorophenyl)-6-[2-(methyloxy)ethyl]-3-azabicyclo[3.1.0]hexane;

(1R,5S,6S/1S,5R,6R)-1-(3,4-dichlorophenyl)-6-[2-(ethyloxy)ethyl]-3-azabicyclo[3.1.0]hexane(1S,5S,6S or 1R,5R,6R)-1-(3,4-dichlorophenyl)-6-[(methyloxy)methyl]-3-azabicyclo[3.1.0]hexane;

(1R,5R,6R or 1S,5S,6S)-1-(3,4-dichlorophenyl)-6-[(methyloxy)methyl]-3-azabicyclo[3.1.0]hexane;

(1S,5S,6S or 1R,5R,6R)-6-{[(cyclopropylmethyl)oxy]methyl}-1-(3,4-dichlorophenyl)-3-azabicyclo[3.1.0];

(1R,5R,6R or 1S,5S,6S)-6-{[(cyclopropylmethyl)oxy]methyl}-1-(3,4-dichlorophenyl)-3-azabicyclo[3.1.0];

([(1S,5S,6S or 1R,5R,6R)]-6-[(methyloxy)methyl]-1-(2-naphthalenyl)-3-azabicyclo[3.1.0]hexane;

([(1R,5R,6R or 1S,5S,6S)]-6-[(methyloxy)methyl]-1-(2-naphthalenyl)-3-azabicyclo[3.1.0]hexane; (1S,5S,6S/1R,5R,6R)-1-(3,4-dichlorophenyl)-6-{[(4-fluorophenyl)oxy]methyl}-3-azabicyclo[3.1.0]hexane;

(1S,5S,6S/1R,5R,6R)-1-(3,4-dichlorophenyl)-6-{[(4-fluorophenyl)oxy]methyl}-3-azabicyclo[3.1.0]hexane;

{[(1S,5S,6S/1R,5R,6R)-1-(3,4-dichlorophenyl)-3-azabicyclo[3.1.0]hex-6-yl]methyl}dimethylamine;

(1S,5S,6S/1R,5R,6R)-1-(3,4-dichlorophenyl)-6-[(methylthio)methyl]-3-azabicyclo[3.1.0]hexane;

(1S,5S,6S or 1R,5R,6R)-1-(3,4-dichlorophenyl)-6-[(methylthio)methyl]-3-azabicyclo[3.1.0]hexane;

(1R,5R,6R or 1S,5S,6S)-1-(3,4-dichlorophenyl)-6-[(methylthio)methyl]-3-azabicyclo[3.1.0]hexane;

(1S,5S,6S or 1R,5R,6R)-1-(3,4-dichlorophenyl)-6-[(methylthio)methyl]-3-azabicyclo[3.1.0]hexane;

(1R,5R,6R or 1S,5S,6S)-1-(3,4-dichlorophenyl)-6-[(methylthio)methyl]-3-azabicyclo[3.1.0]hexane;

or a pharmaceutically acceptable salts, solvates or prodrugs thereof.

In another embodiment, example compounds of the present invention include:

(1S,5S,6S/1R,5R,6R)-6-{[(cyclopropylmethyl)oxy]methyl}-1-(3,4-dichlorophenyl)-3-azabicyclo[3.1.0]hexane;

(1R,5R,6R or 1S,5S,6S)-6-{[(cyclopropylmethyl)oxy]methyl}-1-(3,4-dichlorophenyl)-3-azabicyclo[3.1.0]hexane;

(1S,5S,6S or 1R,5R,6R)-1-(3,4-dichlorophenyl)-6-[(propyloxy)methyl]-3-azabicyclo[3.1.0]hexane;

(1S,5S,6S)-1-(3,4-dichlorophenyl)-6-[(ethyloxy)methyl]-3-azabicyclo[3.1.0]hexane;

(1S,5S,6S/1R,5R,6R)-1-(3,4-dichlorophenyl)-6-{[(2,2,2-trifluoroethyl)oxy]methyl}-3-azabicyclo[3.1.0]hexane;

(1S,5S,6S or 1R,5R,6R)-1-(3,4-dichlorophenyl)-6-{[(2,2,2-trifluoroethyl)oxy]methyl}-3-azabicyclo[3.1.0]hexane;

([(1S,5S,6S/1R,5R,6R)]-6-[(methyloxy)methyl]-1-(2-naphthalenyl)-3-azabicyclo[3.1.0]hexane;

([(1R,5R,6R or 1S,5S,6S)]-6-[(methyloxy)methyl]-1-(2-naphthalenyl)-3-azabicyclo[3.1.0]hexane;

(1R,5R,6R/1S,5S,6S)-1-(3,4-dichlorophenyl)-6-{[(1-methylethyl)oxy]methyl}-3-azabicyclo[3.1.0]hexane;

(1R,5R,6R or 1S,5S,6S)-1-(3,4-dichlorophenyl)-6-{[(1-methylethyl)oxy]methyl}-3-azabicyclo[3.1.0]hexane;

(1R,5R,6R/1S,5S,6S)-6-[(cyclobutyloxy)methyl]-1-(3,4-dichlorophenyl)-3-azabicyclo[3.1.0]hexane;

(1R,5R,6R or 1S,5S,6S)-6-[(cyclobutyloxy)methyl]-1-(3,4-dichlorophenyl)-3-azabicyclo[3.1.0]hexane;

(1S,5S,6S/1R,5R,6R)-1-(3,4-dichlorophenyl)-6-[(methylthio)methyl]-3-azabicyclo[3.1.0]hexane;

(1S,5S,6S or 1R,5R,6R)-1-(3,4-dichlorophenyl)-6-[(methylthio)methyl]-3-azabicyclo[3.1.0]hexane;

(1R,5R,6R/1S,5S,6S)-6-[(cyclopentyloxy)methyl]-1-(3,4-dichlorophenyl)-3-azabicyclo[3.1.0]hexane;

(1R,5R,6R or 1S,5S,6S)-6-[(cyclopentyloxy)methyl]-1-(3,4-dichlorophenyl)-3-azabicyclo[3.1.0]hexane;

(1R,5R,6R/1S,5S,6S)-6-[(cyclohexyloxy)methyl]-1-(3,4-dichlorophenyl)-3-azabicyclo[3.1.0]hexane;

or a pharmaceutically acceptable salts, solvates or prodrugs thereof.

The present invention also provides a process for preparing a compound of formula (I) as above defined.

In the following reaction schemes and hereafter, unless otherwise stated R₁ to R₈, A, K, p, P and n are as defined in the first aspect.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Compounds of formula (Ia), that are compounds of formula (I) wherein R₄ is a methyl group, may be obtained according to Scheme 1, reacting compounds of formula (Ib), i.e. compounds of formula (I) wherein R₄ is hydrogen, through standard reductive amination procedures, for example using formaldehyde and an appropriate reducing agent, such as sodium triacethoxyboronhydride, in protic solvents (such as methanol) at room temperature.

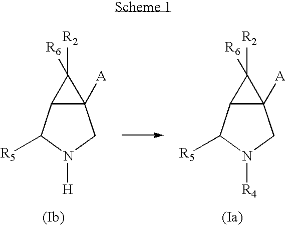

Compounds of formula (Ib), as above defined, may be obtained from compounds of formula (XVI), wherein Pg is a suitable N-protecting group (typically Boc), through deprotection of N-Pg group, (such as BOC using TFA in DCM at temperature between 0° C. and room temperature) according to Scheme 2.

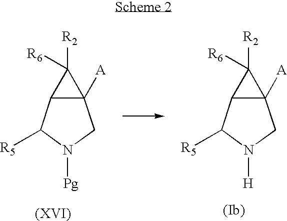

Compounds of formula (XVIa), wherein R₂ is a group P, X is oxygen or sulphur and Pg is a suitable N-protecting group (typically Boc), may be obtained according to Scheme 3 reacting compounds of formula (XVIb), i.e. compounds of formula (XVIa) wherein R₂ is a group P, X is oxygen and R₃ is hydrogen, with methanesulphonyl chloride, in the presence of a trialkylamine base (such as triethylamine), in an aprotic solvent (such as dichloromethane) at temperature comprised between 0° C. and room temperature, followed by displacement of the formed mesyl group, which may be performed by using an alcoholate, such as sodium 2,2,2-trifluoroethanolate (prepared as described in the experimental part) or a thioalcoholate, such as methanethiolate. Alternatively compounds of formula (XVIa), wherein X is oxygen may be obtained according to Scheme 2, starting from compounds of formula (XVIb), as above defined, through standard alkylation procedures, for example using a R₅Y alkylating agent (wherein Y is a leaving group such as an halogen atom), in the presence of a strong base (such as NaH), in an aprotic solvent, (such as DMF), at temperature comprised between 0° C. and room temperature.

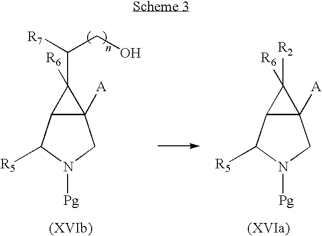

Compounds of formula (XVIc), that are compounds of formula (XVIa) wherein R₇ is methyl and n=0, may be obtained according to Scheme 4, from compounds of formula (II) through oxymercuration-reduction reaction performed with mercuric (II) acetate in THF followed by addition of NaOH and NaBH₄.

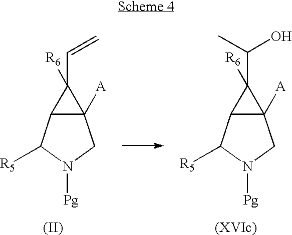

Compounds of formula (II) may be obtained according to Scheme 5 from compounds of formula (III), through a Wittig reaction using methylenetriphenylphosphorane (triphenylphosphine methylide) in THF at room temperature.

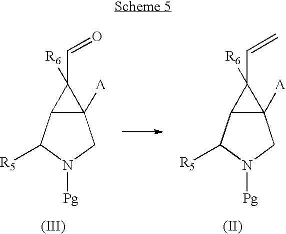

Compounds of formula (III), as described above, may be obtained according to Scheme 6, by oxidation procedure of compounds of formula (XVId), i.e. compounds of formula (XVIb) wherein n is zero and R₇ is hydrogen, for example using Dess-Martin periodinane in DCM at temperature comprised between 0° C. and room temperature.

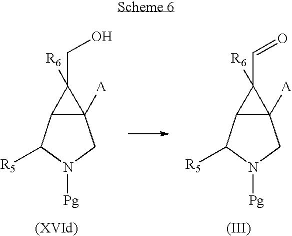

Compounds of formula (XVIe), that are compounds of formula (XVI) wherein R₂ is a group P with n=0, X is nitrogen, R₇ is hydrogen and Pg is a suitable N-protecting group (typically Boc), may be obtained according to Scheme 7, reacting compounds of formula (III), through standard reductive amination procedures, for example using a primary or secondary amine and an appropriate reducing agent, such as sodium triacethoxyboronhydride, in an organic solvent (such as THF) at room temperature.

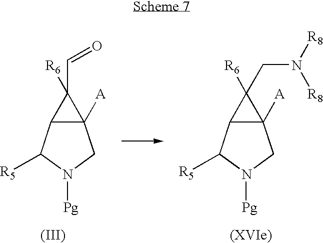

Compounds of formula (XVIf), that are compounds of formula (XVIb) wherein R₂ is a group P with n=1, R₇ is hydrogen, X is oxygen and Pg is a suitable N-protecting group (typically Boc), may be obtained from compounds of formula (II), according to Scheme 8, by hydroboration of the alkene with borane-THF complex in THF at temperature comprised between 0° C. and room temperature followed by oxidation with hydrogen peroxide and NaOH 3.0M at 0° C.

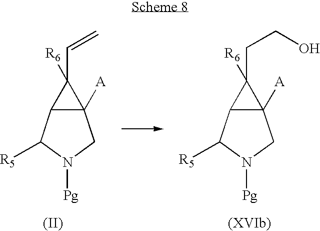

Compounds of formula (XVId), as above defined, may be obtained from compounds of formula (Im), i.e. compounds of formula (I) wherein R₂ is a group P, R₇ si hydrogen, n is zero, X is oxygen and R₃ is hydrogen, according to Scheme 9, through a suitable protecting agent, such as reaction with Boc anhydride, in DCM at temperature between 0° C. and room temperature.

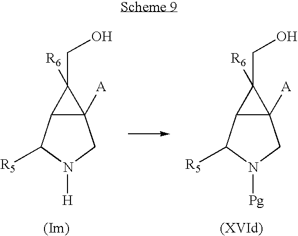

Compounds of formula (In), that are compounds of formula (Im) wherein R5 is hydrogen, may be obtained, according to Scheme 10, starting from compound of formula (V), through an exhaustive reduction procedure using borane-THF complex, in an aprotic solvent (such as THF), at reflux temperature, for the appropriate time, typically comprised between 8 and 12 hours.

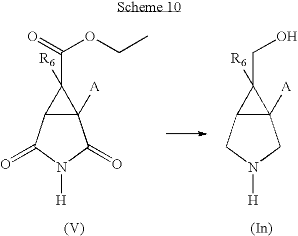

Compounds of formula (Va), i.e. compounds of formula (V) as above defined wherein R₆ is C₁₋₄alkyl, may be obtained, according to Scheme 11 from compounds of formula (VI) through a cycloaddition reaction carried out with the appropriate diazo-derivative (prepared according to the literature procedures, i.e. Org. Biomol. Chem., 2004, 2, 3044-3049) of formula (VII) in an aprotic solvent (such as toluene) heating at reflux temperature for the appropriate time, typically comprised between 2 and 6 hours.

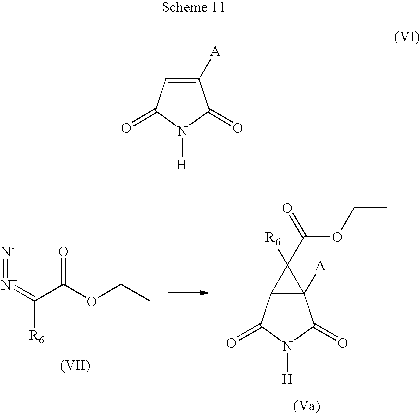

Compounds of formula (Vb), i.e. compounds of formula (V) as above defined wherein R₆ is hydrogen, may be obtained, according to Scheme 12, starting From compound (VIII), wherein Pg is an appropiate protecting group, such as 4-methoxy or 2,4-dimethoxy benzyl. Removal of the methoxy-benzylic protecting group may be carried out through an oxidative cleavage using ceric ammonium nitrate in a mixture of aprotic/protic solvents, such as acetonitrile and water, at room temperature for 12-24 hours.

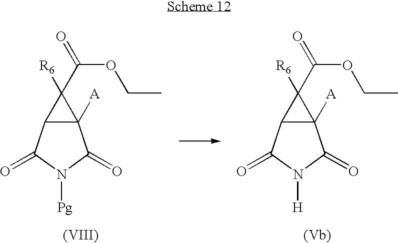

Compounds of formula (VIII), wherein R₆ is hydrogen, may be obtained, according to Scheme 13, starting from compound (IX), through a thermolysis reaction, which may be carried out without any solvent at the appropriate temperature, typically comprised between 180 and 200° C., for 24 h.

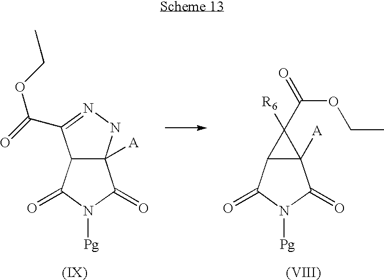

Compounds of formula (IX) may be obtained, according to Scheme 14, starting from compounds of formula (X), wherein Pg is a appropriate protective group as above defined, through a cycloaddition reaction carried out with ethyl diazoacetate, in an aprotic solvent (such as ether or DCM), at room temperature for a period comprised between 5 and 7 days. Alternatively, the reaction can be carried out in toluene, at 100° C. for 24 h.

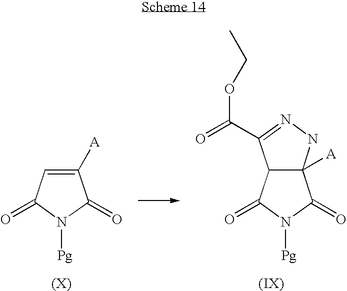

Compounds of formula (X), as above defined, may be obtained, according to Scheme 15, starting from compounds (XI), wherein Pg is a appropriate protective group as defined above, through a modified Suzuki coupling procedure using the appropriate aryl boronic acid, in the presence of a catalyst (such as Pd(PPh₃)₂Cl₂), of a weak base (such as cesium fluoride) and of a promoter of phase transfer (such as benzyltriethylammonium chloride) in a mixture of solvents (for example toluene and water) at a temperature comprised between 60 and 100° C.

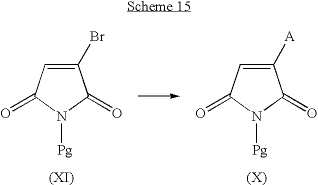

Compounds of formula (XI), as defined above, may be obtained, according to Scheme 16, reacting bromo maleic anhydride (XII) with the appropriate substituted benzyl amine, in protic solvents (for example acetic acid), at reflux temperature for a period between 12 and 16 hours.

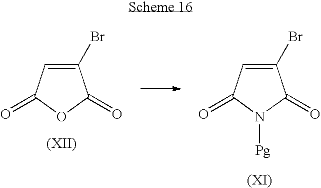

Alternatively, compounds of formula (Vb) wherein R₆ is hydrogen, may be obtained, according to Scheme 17, starting from compounds of formula (VI) through a modified cyclopropanation Corey's procedure, in the presence of a strong base (such as NaH) and in the presence of (ethoxycarbonylmethyl)-dimethylsulfonium in an aprotic solvent (such as DMF), at temperature comprised between 0° C. and room temperature.

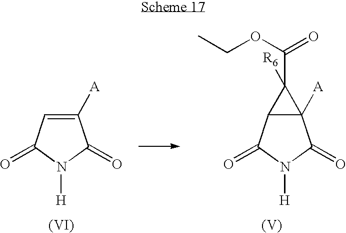

Compounds of formula (VI) may be obtained, according to Scheme 18, starting from maleimide (XIII) through a modified Meerwein arylation carried out in an organic solvent (such as MeCN), in the presence of an agent for the diazotisation (such as tert-butyl nitrite), of substituted aniline and of a radical promoter (such as CuCl₂) in analogy to the method reported in Journal of the American Chemical Society (1956), 78, 6115-20.

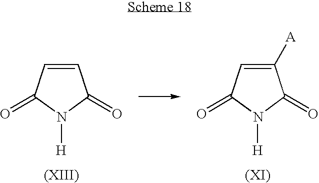

Alternatively, compounds of formula (XVId), as above defined, may be obtained, according to Scheme 19, starting from compound of formula (XIV), through an exhaustive reduction procedure using borane-THF complex, in an aprotic solvent (such as THF), at reflux temperature, for the appropriate time, typically comprised between 8 and 12 hours.

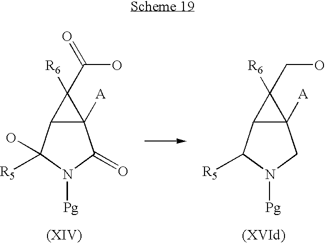

Compounds of formula (XIV) may be obtained, according to Scheme 20, by addition of a Grignard reagent in ethereal solvent at −78° C. to compounds of formula (XV).

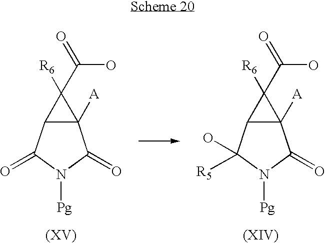

Compounds of formula (XV), as above defined, may be obtained, according to Scheme 21, starting from compound of formula (VIII), through hydrolysis performed under acidic conditions (i.e. HCl 6.0N in acetic acid) and heating to 90° C. for the appropriate time, typically comprised between 12 and 24 hours.

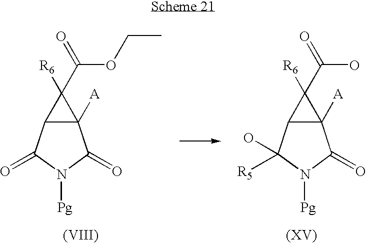

The compounds of the present invention are useful in the treatment of disorders or diseases responsive to the monoamine neurotransmitter re-uptake inhibiting activity of the compounds. This activity of the compounds of the invention may make them useful in the treatment of Parkinsonism, depression, eating disorders, sleep disorders, substance related disorders, attention-deficit hyperactivity disorders, anxiety disorders, cognition impairment, sexual dysfunctions, obsessive compulsive spectrum disorders, Gilles de la Tourettes disease and senile dementia, as well as other disorders sensitive to the monoamine neurotransmitter re-uptake-inhibiting activity of the compounds.

Within the context of the present invention, the terms describing some indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

The term “depression” includes:

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90): Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80);

The term “anxiety disorders” includes:

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):

The term “substance related disorder” includes:

Substance-related disorders including Substance Use Disorders such as Substance Dependence, Substance Craving and Substance Abuse; Substance-induced Disorders such as Substance Intoxication, Substance Withdrawal, Substance-induced Delirium, Substance-Induced Persisting Dementia, Substance-Induced Persisting Amnestic Disorder, Substance-induced Psychotic Disorder, Substance-induced Mood Disorder, Substance-Induced Anxiety Disorder, Substance-induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-induced Mood Disorder, Amphetamine-Induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-Induced Mood Disorder, Cocaine-induced Anxiety Disorder, Cocaine-induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide;

The term “Sleep disorder” includes:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type;

The term “eating disorder” include:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):

The term “Attention-Deficit/Hyperactivity Disorder” includes:

Attention-Deficit/Hyperactivity Disorder including the subtypes Attention-Deficit /Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23);

The term “Cognition impairment” includes:

Cognition impairment including cognition impairment in other diseases such as schizophrenia, bipolar disorder, depression, other psychiatric disorders and psychotic conditions associated with cognitive impairment, e.g. Alzheimer's disease;

The term “Sexual dysfunctions” includes:

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9);

The term “Obsessive compulsive spectrum disorder” includes:

Obsessive compulsive spectrum disorder including Obsessive compulsive disorders (300.3), somatoform disorders including body dysmorphic disorder (300.7) and hyperchondriasis (300.7), bulimia nervosa (307.51), anorexia nervosa (307.1), eating disorders not elsewhere classified (307.50) such as binge eating, impulse control disorders not elsewhere classified (including intermitted explosive disorder (312.34), compulsive buying or shopping, repetitive self-mutilation, onychophagia, psychogenic excoriation, kleptomania (312.32), pathological gambling (312.31), trichotillomania (312.39) and internet addiction), paraphilia (302.70) and nonparaphilic sexual addictions, Sydeham's chorea, torticollis, autistic disorders (299.0), compulsive hoarding, and movement disorders, including Tourette's syndrome (307.23).

All of the various forms and sub-forms of the disorders mentioned herein are contemplated as part of the present invention.

In an embodiment, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis); musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the amelioration of inflammatory disorders, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastroesophageal reflux disease); other conditions with an inflammatory component such as migraine, multiple sclerosis, myocardial ischemia.

In one embodiment, compounds of the invention are useful in the treatment of depression and anxiety disorders.

In another embodiment, compounds of the invention are useful in the treatment of depression.

“Treatment” includes prophylaxis, where this is appropriate for the relevant condition(s).

In an alternative or further aspect there is provided a method for the treatment of a mammal, including man, in particular in the treatment of disorders or diseases responsive to the monoamine neurotransmitter re-uptake inhibiting activity of the compounds, comprising administration of an effective amount of a compound of the invention.

In one embodiment, the invention provides a method of treating a condition for which inhibition of serotonin (5-HT), dopamine (DA) and norepinephrine (NE), is beneficial, which comprises administering to a mammal (e.g. human) in need thereof an effective amount of a compound of the invention.

In another aspect, the invention provides a compound of the invention for use in therapy.

In a further embodiment, the invention provides a compound of the invention for use in the treatment of a condition in a mammal for which inhibition of serotonin (5-HT), dopamine (DA) and norepinephrine (NE) is beneficial.

In one aspect, the invention provides the use of compounds of the invention, for the manufacture of a medicament for the treatment of disorders or diseases responsive to monoamine neurotransmitter re-uptake inhibiting activity.

In one embodiment, the invention provides the use of a compound of a compound of the invention in the manufacture of a medicament for the treatment of a condition in a mammal for which inhibition of serotonin (5-HT), dopamine (DA) and norepinephrine (NE) is beneficial.

The compounds of the invention may also be used in combination with other therapeutic agents. The invention thus provides, in a further aspect, a combination comprising a compound of the invention together with a further therapeutic agent.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilizers; ii) antipsychotics; and iii) antidepressants.

The compounds of the invention may be used in combination with the following agents to treat or prevent anxiety disorders: i) anxiolytics; and ii) antidepressants.

The compounds of the invention may be used in combination with the following agents to improve nicotine withdrawal and reduce nicotine craving: i) nicotine replacement therapy for example a sublingual formulation of nicotine beta-cyclodextrin and nicotine patches; and ii) bupropion.

The compounds of the invention may be used in combination with the following agents to improve alcohol withdrawal and reduce alcohol craving: i) NMDA receptor antagonists for example acamprosate; ii) GABA receptor agonists for example tetrabamate; and iii) Opioid receptor antagonists for example naltrexone.

The compounds of the invention may be used in combination with the following agents to improve opiate withdrawal and reduce opiate craving: i) opioid mu receptor agonist/opioid kappa receptor antagonist for example buprenorphine; ii) opioid receptor antagonists for example naltrexone; and iii) vasodilatory antihypertensives for example lofexidine.

The compounds of the invention may be used in combination with the following agents to treat or prevent sleeping disorders: i) benzodiazepines for example temazepam, lormetazepam, estazolam and triazolam; ii) non-benzodiazepine hypnotics for example zolpidem, zopiclone, zaleplon and indiplon; iii) barbiturates for example aprobarbital, butabarbital, pentobarbital, secobarbita and phenobarbital; iv) antidepressants; v) other sedative-hypnotics for example chloral hydrate and chlormethiazole.

The compounds of the invention may be used in combination with the following agents to treat anorexia: i) appetite stimulants for example cyproheptidine; ii) antidepressants; iii) antipsychotics; iv) zinc; and v) premenstral agents for example pyridoxine and progesterones.

The compounds of the invention may be used in combination with the following agents to treat or prevent bulimia: i) antidepressants; ii) opioid receptor antagonists; iii) antiemetics for example ondansetron; iv) testosterone receptor antagonists for example flutamide; v) mood stabilizers; vi) zinc; and vii) premenstral agents.

The compounds of the invention may be used in combination with the following agents to treat or prevent autism: i) antipsychotics; ii) antidepressants; iii) anxiolytics; and iv) stimulants for example methylphenidate, amphetamine formulations and pemoline.

The compounds of the invention may be used in combination with the following agents to treat or prevent ADHD: i) stimulants for example methylphenidate, amphetamine formulations and pemoline; and ii) non-stimulants for example norepinephrine reuptake inhibitors (such as atomoxetine), alpha 2 adrenoceptor agonists (such as clonidine), antidepressants, modafinil, and cholinesterase inhibitors (such as galantamine and donezepil).

The compounds of the invention may be used in combination with the following agents to treat personality disorders: i) antipsychotics; ii) antidepressants; iii) mood stabilizers; and iv) anxiolytics.

The compounds of the invention may be used in combination with the following agents to treat or prevent male sexual dysfunction: i) phosphodiesterase V inhibitors, for example vardenafil and sildenafil; ii) dopamine agonists/dopamine transport inhibitors for example apomorphine and buproprion; iii) alpha adrenoceptor antagonists for example phentolamine; iv) prostaglandin agonists for example alprostadil; v) testosterone agonists such as testosterone; vi) serotonin transport inhibitors for example serotonin reuptake inhibitors; v) noradrenaline transport inhibitors for example reboxetine and vii) 5-HT1A agonists, for example flibanserine.

The compounds of the invention may be used in combination with the same agents specified for male sexual dysfunction to treat or prevent female sexual dysfunction, and in addition an estrogen agonist such as estradiol.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine and sertraline); dual serotonin/noradrenaline reuptake inhibitors (such as venlafaxine, duloxetine and milnacipran); Noradrenaline reuptake inhibitors (such as reboxetine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabilizer drugs include lithium, sodium valproate/valproic acid/divalproex, carbamazepine, lamotrigine, gabapentin, topiramate and tiagabine.

Anxiolytics include benzodiazepines such as alprazolam and lorazepam.

For use in medicine, the compounds of the present invention are usually administered as a standard pharmaceutical composition. The present invention therefore provides in a further aspect a pharmaceutical composition comprising a compound of the invention and a pharmaceutically (i.e physiologically) acceptable carrier. The pharmaceutical composition can be for use in the treatment of any of the conditions described herein.

The compounds of the invention may be administered by any convenient method, for example by oral, parenteral (e.g. intravenous), buccal, sublingual, nasal, rectal or transdermal administration and the pharmaceutical compositions adapted accordingly.

The compounds of the invention which are active when given orally can be formulated as liquids or solids, for example syrups, suspensions or emulsions, tablets, capsules and lozenges.

A liquid formulation will generally consist of a suspension or solution of the compound or salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or salt in a sterile aqueous carrier or parenterally acceptable oil, for example polyethylene glycol, polyvinyl pyrrolidone, lecithin, arachis oil or sesame oil. Alternatively, the solution can be lyophilized and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluoro-chlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment, the composition is in unit dose form such as a tablet, capsule or ampoule.

Each dosage unit for oral administration contains for example from 0.5 to 250 mg (and for parenteral administration contains for example from 0.05 to 25 mg) of a compound of the invention calculated as the free base.

The pharmaceutically acceptable compounds of the invention will normally be administered in a daily dosage regimen (for an adult patient) of, for example, an oral dose of between 1 mg and 500 mg, for example between 1 mg and 400 mg, e.g. between 10 and 250 mg or an intravenous, subcutaneous, or intramuscular dose of between 0.1 mg and 100 mg, for example between 0.1 mg and 50 mg, e.g. between 1 and 25 mg of the compound of the formula (I) or a salt thereof calculated as the free base, the compound being administered 1 to 4 times per day, for example 1 to 2 time a day. In one embodiment, the compound of the invention may be administered once a day. Suitably the compounds will be administered for a period of continuous therapy, for example for a week or more.

For oral administration a typical dose may be in the range of 1 to 200 mg per day, for example 60 to 200 mg per day.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

The invention is also directed to a novel kit-of-parts that is suitable for use in the treatment of disorders as above defined comprising a first dosage form comprising a compound of the invention and a second dosage form comprising another therapeutic agent, for simultaneous, separate or sequential administration.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

Biological Assays

Cell Biology

a) Generation of BacMam Viruses for the Expression of hSERT, hNET, and hDAT in Mammalian Cells

Membranes for the SPA-binding assays are produced by HEK-293F cell infection with BacMam viruses generated for each single human SERT, NET, and DAT transporter. hSERT and hDAT are cloned into pFBMRfA vector whereas hNET is cloned into pFASTBacMam1 vector. The generation and use of BacMam viruses is described in Condreay J P et al, Proc. Natl. Acad. Sci. USA, 1999, 96:127-132 and Hassan N J et al, Protein Expression and Purification, 47(2): 591-598, 2006.

Affinity to the Human Transporters SERT, NET and DAT

The affinities of the compounds of the invention for the human serotonin transporter (SERT), human norepinephrine transporter (NET) and for the human dopamine transporter (DAT) may be determined by the assays described below. Such affinity is typically calculated from the IC₅₀ obtained in competition experiments as the concentration of a compound necessary to displace 50% of the radiolabeled ligand from the transporter, and is reported as a “Kᵢ” value calculated by the following equation:

$${K}_{i} = \frac{{IC}_{50}}{1 + L / {K}_{D}}$$

where L=radioligand and K_{D}=affinity of radioligand for transporter (Cheng and Prusoff, Biochem. Pharmacol. 22:3099, 1973). In the context of the present invention pKi values (corresponding to the antilogarithm of Ki) are used instead of Ki; pKi results are only estimated to be accurate to about 0.3-0.5.

a) Scintillation Proximity Assay (SPA) for Human DAT, NET and SERT Binding

Transduction of HEK-293F cells with hSERT/hDAT/hNET BacMam viruses

The HEK-293F suspension cell line (Invitrogen) is routinely grown in 293_Freestyle Expression media (Invitrogen) in shake flask suspension culture. The culture is transduced with the appropriate transporter BacMam at a MOI (multiplicity of infection) of 100 virus particles per cell and incubated for 48 hrs at 37° C., 5% CO₂ in air, shaken at 90 rpm in a humidified shaker incubator. The culture is then harvested by centrifugation at 1000 g, 4° C., for 10 minutes and the cell pellet stored at −80° C. until required.

Preparation of BacMam hSERT/hDAT/hNET-HEL293F Cell Membranes

Transduced cell pellets are re-suspended to 10× volume with buffer-A (50 mM HEPES, 1 mM EDTA, 1 mM leupeptin, 25 ug/mL bacitracin, 1 mM phenylmethylsulfonylfluoride, PMSF, 2 μM pepstatin A, pH 7.7) and homogenized with 2×15 second bursts in a glass Waring blender. The homogenate is then centrifuged for 20 minutes at 500 g. Following this, the supernatant is pooled and centrifuged at 13,000 g for 30 minutes. Pellets are then re-suspended to 4× original pellet volume with buffer-B (50 mM TRIS pH 7.4, 130 mM NaCl) and forced through a 0.8 mm needle to give a homogeneous suspension. Membrane aliquots are stored at −80° C. until required. The protein concentration is quantified by Bradford assay.

SPA-Binding Assay Protocol for hSERT, hNET, and hDAT

The affinity of the compounds of the invention to the hSERT, hNET or hDAT can be also assessed by using the [³H]citalopram, [³H]nisoxetine or [³H]WIN-35,428 binding assays with the SPA technology on BacMam-recombinant human SERT, NET and DAT membranes produced as described before. With the SPA technology (GE Healthcare, Amersham) only transporter-bound radioactivity can elicit bead excitation thus no separation of the bound/unbound radioligand is required.

The protocol for hSERT binding SPA is based on Trilux beta-counter (Wallac, Perkin-Elmer). Briefly, 0.5 μL of test compound in neat DMSO (or 1 μM fluoxetine as positive control) is added by 50 μL of the SPA mixture, containing 2 mg/mL SPA beads (Amersham RPNQ0001), 4 μg/mL hSERT Bacmam membranes, 0.01% pluronic F-127, 2.5 nM [³H]citalopram in the assay buffer (20 mM HEPES, 145 mM NaCl, 5 mM KCl, pH 7.3). Incubation are performed at room temperature for at least 2 hours. Counts are stable and could be read up to 3 days.

Alternatively, hDAT hNET and hSERT SPA-binding assays are performed by using a Viewlux beta-counter (Wallac, Perkin-Elmer) with imaging PS-WGA beads (Amersham RPNQ0260) in a final assay volume of 30 μL and in a 384-well plate format (Greiner 781075). Briefly, 0.3 μL of test compound in neat DMSO and 0% and 100% effect controls (DMSO for total binding and 10 or 1 μM indatraline as positive control) are added to the wells by using a Hummingbird (Genomic Solutions), followed by the addition of 30 μL of the SPA mixture, containing 1 mg/mL SPA beads (hSERT) or 2 mg/ml SPA beads (hDAT and hNET), 40 μg/ml or 20 μg/ml or 6 μg/ml of hDAT or hNET or hSERT BacMam membranes, 0.02% pluronic F-127, 10 nM [³H]WIN-35,428 or 10 nM [³H]nisoxetine or 3 nM [³H]citalopram for hDAT or hNET or hSERT binding SPA in the assay buffer (20 mM HEPES, 145 mM NaCl, 5 mM KCl, pH 7.3-7.4). Incubation is performed at room temperature for at least 2 hours, best overnight in the dark. Bound radioactivity is recorded by using a 600 s 6× binning and 613 nm emission filter with the Viewlux instrument.

Compound Affinity Range for Human Transporters SERT, NET, and DAT

The compounds of formula (I)′ typically show pKi greater than 4.5 towards each of the three transporters SERT, NET and DAT. In one embodiment, the compounds of formula (I)′ typically show pKi greater than 5.5 for each of the three transporters. In another embodiment, the compounds of formula (I)′ typically show pKi greater than 6.5 for each of the three transporters. In a further embodiment, the compounds of formula (I)′ typically show pKi greater than 7.5 for each of the three transporters.

In one embodiment, the present invention provides compounds of formula (I)′ having a hSERT pKi comprised between 7 and 8.5. In another embodiment, the present invention provides compounds of formula (I)′ having a hSERT pKi comprised between 8.5 and 10.

In one embodiment, the present invention provides compounds of formula (I) having a hDAT pKi comprised between 6.5 and 7.5. In another embodiment, the present invention provides compounds of formula (I)′ having a hDAT pKi comprised between 7.5 and 9.

In one embodiment, the present invention provides compounds of formula (I)′ having a hNET pKi comprised between 6.5 and 7.5. In another embodiment, the present invention provides compounds of formula (I)′ having a hNET pKi comprised between 7.5 and 10.

In one embodiment, the present invention provides compounds of formula (I)′ having a a hSERT pKi comprised between 8.5 and 10, a hNET pKi comprised between 7.5 and 20 and a hDAT pKi comprised between 7.5 and 9.

In one embodiment, the present invention provides compounds of formula (I)′ having a hSERT pKi comprised between 9 and 10, a hNET pKi comprised between 8.0 and 8.5 and a hDAT pKi comprised between 7.5 and 8.0.

### EXAMPLES

The invention is further illustrated by the following non-limiting examples.

In the procedures that follow, after each starting material, reference to a Description or Example by number is typically provided. This is provided merely for assistance to the skilled chemist. The starting material may not necessarily have been prepared from the batch referred to.

Where reference is made to the use of a “similar” or “analogous” procedure, as will be appreciated by those skilled in the art, such a procedure may involve minor variation, for example reaction temperature, reagent/solvent amount, reaction time, work-up conditions or chromatographic purification conditions.

In the procedures that follow, the use of dotted or bold bond in the graphical representation of molecules is not intended to provide any indication on the absolute configuration of stereogenic centers but to provide the relative disposition in the space of substituents attached to the [3.1.0]azabicyclic ring.

For example, in Example 2 shown below, the C-5 hydrogen atom and the C1-3,4.dichlorophenyl ring are intended to be on opposite faces of the cyclopropane ring with respect to the C-6 —CH₂OH group (generating stereochemistry ENDO), while in Example 1 shown below they are intended to be on the same face of the cyclopropane ring (generating stereochemistry EXO).

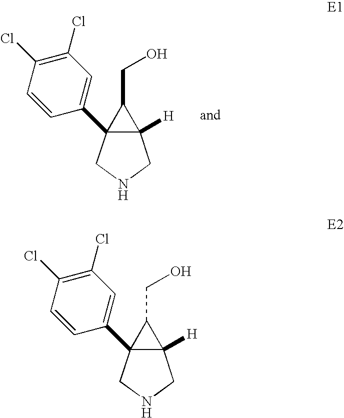

Absolute stereochemistry, if available, is provided by absolute configuration of stereogenic centers indicated in names of compounds.

Wherein the compounds' name only refers to absolute configuration of stereogenic centers by quoting opposite configurations divided by a slash sign, these are to be intended as 1:1 mixtures of the corresponding stereosiomers, actually being a racemic mixture [for example, (1S,5S,6S/1R,5R,6R) represents a mixture of (1S,5S,6S) and (1R,5R,6R) stereoisomers)].

Proton Magnetic Resonance (NMR) spectra are typically recorded either on Varian instruments at 300, 400 or 500 MHz, or on a Bruker instrument at 300 and 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is reported.

Mass spectra (MS) are typically taken on a 4 II triple quadrupole Mass Spectrometer (Micromass UK) or on a Agilent MSD 1100 Mass Spectrometer, operating in ES (+) and ES (−) ionization mode or on an Agilent LC/MSD 1100 Mass Spectrometer, operating in ES (+) and ES (−) ionization mode coupled with HPLC instrument Agilent 1100 Series [LC/MS-ES (+):analysis performed on a Supelcosil ABZ+Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [CH₃CN ] in 5 min, finally under these conditions for 2 min; T=40° C.; flux=1 mL/min; LC/MS-ES (−):analysis performed on a Supelcosil ABZ+Plus (33×4.6 mm, 3 μm) (mobile phase: 100% [water+0.05% NH₃] for 1 min, then from 100% [water+0.05% NH₃ to 5% [water+0.05% NH₃] and 95% [CH₃CN ] in 5 min, finally under these conditions for 2 min; T=40° C.; flux=1 mL/min]; in the mass spectra only one peak in the molecular ion cluster is reported.

DAD chromatographic traces, mass chromatograms and mass spectrums may be taken on a on a UPLC/MS Acquity™ system coupled with a Micromass ZQ™ mass spectrometer operating in ESI positive or negative. The phases used are: A) H2O/ACN 95/5+0.1% TFA; B) H2O/ACN 5/95+0.1% TFA. The gradient is: t=0 min) 95% A 5% B, t=0.25) 95% A 5% B, t=3.30) 100% B, t=4.0) 100% B, followed by 1 min of reconditioning. Compounds are named using ACD/Name PRO 6.02 chemical naming software (Advanced Chemistry Development Inc., Toronto, Ontario, M5H2L3, Canada).

Flash silica gel chromatography was carried out on silica gel 230-400 mesh (supplied by Merck AG Darmstadt, Germany) or over Varian Mega Be—Si pre-packed cartridges or over pre-packed Biotage silica cartridges.

SPE-SCX cartridges are ion exchange solid phase extraction columns supplied by Varian. The eluent used with SPE-SCX cartridges is methanol followed by 2N ammonia solution in methanol.

In a number of preparations, purification was performed using either Biotage manual flash chromatography (Flash+) or automatic flash chromatography (Horizon, SP1) systems. All these instruments work with Biotage Silica cartridges.

SPE-Si cartridges are silica solid phase extraction columns supplied by Varian.

The following abbreviations are used in the text: AcOH=Acetic acid, EtOAc=ethyl acetate, DCM=dichloromethane, Et₂O=dietyl ether, THF=tetrahydrofuran, TFA=trifluoroacetic acid, MeOH=Methanol, DMSO=dimethylsulfoxide, DMF=N,N-dimethylformamide, TEA=triethylamine, Boc2O=di-t-butyldicarbonate, SCX=strong cation exchanger, and dried refers to a solution dried over anhydrous sodium sulphate, r.t. (RT) refers to room temperature, Rt=retention time; h=hour, FC=flash chromatography, NH column: secondary amine functionalized silica cartridge.

Enantiomer 1 or Enantiomer 2 refers to a single enantiomer whose absolute stereochemistry was not characterized.

### Preparation 1: 3-(3,4-dichlorophenyl)-1H-pyrrole-2,5-dione (P1)

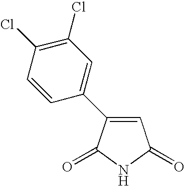

To a stirred slurry of maleimide (10 g), anhydrous CuCl₂ (10 g) and tert-butyl nitrite (11 mL) in CH₃CN (300 mL) at 0° C. a solution of 3,4-dichloro aniline (10 g) in CH₃CN (100 mL) was added dropwise. The reaction mixture was stirred at room temperature for 1 h and 20% aqueous HCl (500 mL) was added. The mixture was extracted with ethyl acetate, the organic layer was washed with saturated aqueous NaCl and dried over Na₂SO₄. To a solution of the crude obtained in isopropanole (100 mL) 2,6-lutidine (7 mL) was added and the mixture was warmed at reflux for 30 minutes. After elimination of the solvent under vacuum, the crude was dissolved in ethyl acetate and the organic phase washed with water and dried over sodium sulphate. The organic phase was concentrated under vacuum and the crude treated with diethyl ether. The solid was filtrated and dried under vacuum to give the title compound in 2.59 g yield as brown solid.

MS (m/z): 241 [M-H]⁻.

### Preparation 2: ethyl[(1S,5S,6S/1R,5R,6R) and (1S,5S,6R/1R,5R,6S)]-1-(3,4-dichlorophenyl)-2,4-dioxo-3-azabicyclo[3.1.0]hexane-6-carboxylate (P2)

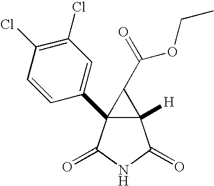

Sodium hydroxide 60% in mineral oil (0.66 g) was added in small portions to a stirred solution of (ethoxycarbonylmethyl)-dimethylsulfonium bromide (5 g) in anhydrous DMSO (20 mL). The resulting mixture was allowed to stir at room temperature for 1.5 h then 3-(3,4-dichlorophenyl)-1H-pyrrole-2,5-dione (P1, 2 g) dissolved in DMSO (20 mL) was added dropwise and the resulting mixture was stirred at room temperature for 1 minutes. Reaction temperature was brought to 0° C. and aqueous saturated NH₄Cl (80 mL) was slowly added, followed by Et₂O (100 mL). After separation of the two phases, the organic layer was washed twice with water (2×60 mL), brine (1×60) and dried over Na₂SO₄. Evaporation of the solvent under vacuum gave a crude compound which was purified by flash chromatography (eluting with ethyl acetate/cycloesane 20:80) to give 780 mg of the title compound.

MS (m/z): 326 [M-H]⁻. Stereochemistry complex, not assigned.

### Preparation 3: 3-bromo-1-{[4-(methyloxy)phenyl]methyl}-1H-pyrrole-2,5-dione (P3)

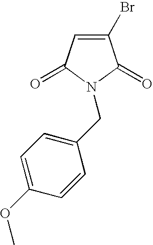

A mixture of 3-bromo-2,5-furandione (6 g), 1-[4-(methyloxy)phenyl]methanamine (4.44 mL), and AcOH (80 mL) was heated at 100° C. overnight. The solution was then concentrated in vacuo. AcOH (70 mL) and AcONa (2 g) were added to the crude product and the mixture was reflux for 2 hours. Water was then added and the aqueous phase was extracted with DMC. The organic phase was dried and evaporated in vacuo. The crude was purified by flash chromatography eluting with cyclohexane/ethyl acetate from 9/1 to 7/3 to give the title compound (8.2 g).

NMR (¹H, CDCl₃) δ 7.32 (d, 2H), 6.86 (m, 3H), 4.66 (s, 2H), 3.80 (s, 3H).

### Preparation 4: 3-(3,4-dichlorophenyl)-1-{[4-(methyloxy)phenyl]methyl}-1H-pyrrole-2,5-dione (P4)

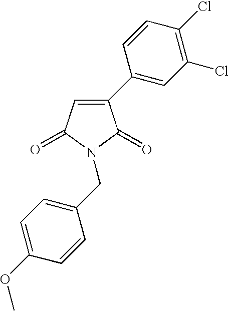

A solution of 3-bromo-1-{[4-(methyloxy)phenyl]methyl}-1H-pyrrole-2,5-dione (P3, 4 g), (3,4-dichlorophenyl)boronic acid (5.16 g), Pd(PPh₃)₂Cl₂ (1.028 g), cesium fluoride (5.54 g) and benzyltriethylammonium chloride (307 mg), in a mixture of solvents such as toluene/H₂O 1:1, was heated at 90° C. for 19 h. The solution was then concentrated in vacuo. Dichloromethane was added and the organic phase was washed with aqueous saturated NH₄Cl solution. The organic layer was dried and evaporated in vacuo. Et₂O was added to the crude and the precipitate was filtrated and then the filtrate evaporated in vacuo. The crude product was purified by flash chromatography eluting with cyclohexane/ethyl acetate from 9/1 to 8/2 to give the title compound (1.93 g).

NMR (¹H, CDCl₃) δ 8.09 (s, 1H), 7.77 (d, 1H), 7.54 (d, 1H), 7.36 (d, 2H), 6.88 (d, 2H), 6.77 (s, 1H), 4.69 (s, 2H), 3.81 (s, 3H).

### Preparation 5: ethyl 6a-(3,4-dichlorophenyl)-5-{[4-(methyloxy)phenyl]methyl}-4,6-dioxo-1,3a,4,5,6,6a-hexahydropyrrolo[3,4-c]pyrazole-3-carboxylate (P5)

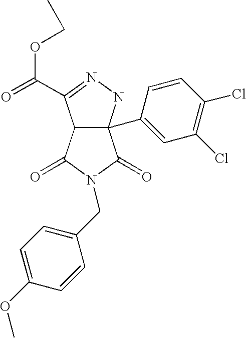

A solution of 3-(3,4-dichlorophenyl)-1-{[4-(methyloxy)phenyl]methyl}-1H-pyrrole-2,5-dione (P4, 1.93 g) and ethyldiazoacetate (0.61 mL) in DCM (15 mL) was stirred at room temperature for 4 days. The solvent was removed under reduced pressure and the residue was purified by flash chromatography eluting with cyclohexane/ethyl acetate from 9/1 to 8/2 to give the title compound (1.84 g).

NMR (¹H, CDCl₃) δ 7.45 (d, 1H), 7.34 (d, 2H), 7.17 (s, 1H), 7.01 (d, 1H), 6.87 (d, 2H), 4.72 (s, 2H), 4.39 (m, 2H), 3.82 (s, 3H), 1.39 (t, 3H). MS (m/z): 476 [MH]⁺.

### Preparation 6: ethyl(1S,5S,6S/1R,5R,6R or 1S,5S,6R/1R,5R,6S )-1-(3,4-dichlorophenyl)-2,4-dioxo-3-azabicyclo[3.1.0]hexane-6-carboxylate (P6)

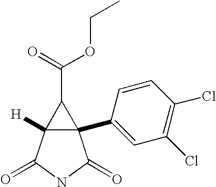

Ethyl-6a-(3,4-dichlorophenyl)-5-{[4-(methyloxy)phenyl]methyl}-4,6-dioxo-1,3a,4,5,6,6a-hexahydropyrrolo[3,4-c]pyrazole-3-carboxylate (P5, 1.84 g) was heated at 200° C. for 20 h. The crude product was purified by flash chromatography eluting with cyclohexane/ethyl acetate from 9/1 to 8/2 to give ethyl 1-(3,4-dichlorophenyl)-2,4-dioxo-3-azabicyclo[3.1.0]hexane-6-carboxylate (1.03 g).

NMR (¹H, CDCl₃) δ 7.55 (s, 1H), 7.46 (d, 1H), 7.27 (m, 3H), 6.85 (d, 2H), 4.53 (dd, 2H), 3.98 (q, 2H), 3.80 (s, 3H), 3.41 (m 1H), 2.59 (s, 1H), 1.06 (t, 3H).

A mixture of this material (0.98 g) and ceric ammonium nitrate (2.64 g), in CH₃CN/H₂O 1:1 was stirred at r.t. overnight. The solution was concentrated in vacuo and then the ethyl acetate was added to the mixture. The organic phase was separated and washed with aqueous saturated solution NaCl. The organic layer was dried and evaporated in vacuo. The crude was purified by flash chromatography eluting with cyclohexane/ethyl acetate from 9/1 to 8/2 to give the title compound (380 mg).

NMR (¹H, CDCl₃) δ 7.56 (s, 1H), 7.48 (d, 1H), 7.32 (m, 1H), 4.02 (q, 2H), 3.42 (s, 1H), 2.92 (s, 1H), 1.10 (t, 3H).

The Title product was submitted to chromatography analysis by UPLC (ultra performance liquid chromatography)

UPLC Waters Acquity™ system,coupled with Waters ZQ™ single quadrupole MS detector. The system acquire UV (DAD 210-350 nm Wavelength range) and Total Ion Current (TIC) MS chromatographic traces using ES⁺ (100-1000 amu) or ES⁻ (100-800 amu) ionisation modes.

Column: Acquity™ BEH C18 2.1×50 mm 1.7 μm. (column temperature 40° C.)

Flow rate 1 ml/min.

Mobile phase: A=H2O+0.1% formic acid and B=MeCN+0.06% formic acid.

Gradient timetable: time 0 min 3% B to 6% B in 0.05 min, to 70% B in 0.52 min and to 99% in further 0.49 min lasting for 0.39 min. At time 1.45 min the gradient composition become 97% A/3% B (initial conditions) and last for 0.05 min (stop time=1.50 min).

Rt=0.72 min; MS (m/z): 326 [M]⁻.

### Preparation 7: 1-{[2,4-bis(methyloxy)phenyl]methyl}-3-bromo-1H-pyrrole-2,5-dione (P7)

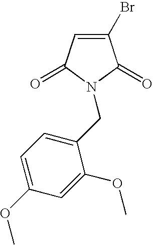

The title compound was prepared as yellow oil in 2.7 g yield from 1-[2,4-bis(methyloxy)phenyl]methanamine (2.58 mL) using a similar procedure as set out earlier described in Preparation 3.

MS (m/z): 327 [MH]⁺.

### Preparation 8: 1-{[2,4-bis(methyloxy)phenyl]methyl}-3-(2-naphthalenyl)-1H-pyrrole-2,5-dione (P8)

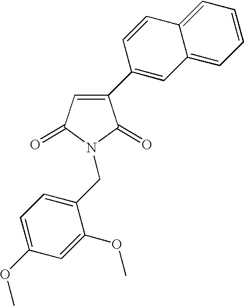

The title compound was prepared as yellow oil in 0.78 g yield from 1-{[2,4-bis(methyloxy)phenyl]methyl}-3-bromo-1H-pyrrole-2,5-dione (P7) and 2-naphthalenyl boronic acid (2.13 g) using a similar procedure as set out earlier described in Preparation 4.

MS (m/z): 374 [MH]⁺.

### Preparation 9: ethyl(1S,5S,6S/1R,5R,6R or 1S,5S,6R/1R,5R,6S )-1-(2-naphthalenyl)-2,4-dioxo-3-azabicyclo[3.1.0]hexane-6-carboxylate (P9)

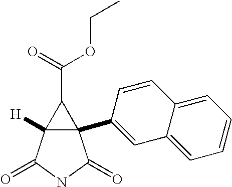

A solution of 3-(2-naphthalenyl)-1-{[2,4-bis(methyloxy)phenyl]methyl}-1H-pyrrole-2,5-dione (P8, 0.78 g) and ethyldiazoacetate (1.1 mL) in toluene (15 mL) was stirred at 100° C. for 18 h. The solvent was removed under reduced pressure and the residue was heated at 200° C. for 20 h. The crude product was purified by flash chromatography eluting with cyclohexane/ethyl acetate from 9/1 to give ethyl(1S,5S,6S/1R,5R,6R or 1S,5S,6R/1R,5R,6S)-3-{[2,5-bis(methyloxy)phenyl]methyl}-1-(2-naphthalenyl)-2,4-dioxo-3-azabicyclo[3.1.0]hexane-6-carboxylate (200 mg).

NMR (¹H, CDCl₃) δ 7.91 (d, 1H), 7.84 (m, 3H), 7.53-7.50 (m, 3H), 7.18 (d, 1H), 6.46 (m, 2H), 4.68 (d, 1H), 4.528 (d, 1H), 3.88-3.82 (m, 5H), 3.54 (d, 1H), 2.71 (d, 1H), 0.90 (t, 3H). MS (m/z): 460 [MH]⁺.

To a mixture of this material (0.20 g) in acetonitrile (3 mL) ceric ammonium nitrate (0.54 g), in water (3 mL) was added and the mixture was stirred at room temperature.t. overnight. After that a solution of ceric ammonium nitrate (0.12 g) in water (0.5 mL) was added to the reaction mixture and the solution was stirred at room temperature for 4 h. The solution was concentrated in vacuo and dichloromethane was added to the mixture. The organic phase was separated, dried over sodium sulphate and evaporated in vacuo. The crude was purified by flash chromatography eluting with cyclohexane/ethyl acetate 8/2 to give the title compound (105 mg).

NMR (¹H, CDCl₃) δ 7.91-7.85 (ms, 4H), 7.54-7.52 (m, 3H), 3.93-3.88 (q, 2H), 3.56 (d, 1H), 3.01 (d, 1H), 0.94 (t, 3H).

### Preparation 10: [(1S,5S,6S/1R,5R,6R or 1S,5S,6R/1R,5R,6S )-1-(2-naphthalenyl)-3-azabicyclo[3.1.0]hex-6-yl]methanol (P10)

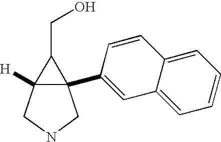

To a stirred solution of ethyl(1S,5S,6S/1R,5R,6R or 1S,5S,6R/1R,5R,6S )-1-(2-naphthalenyl)-2,4-dioxo-3-azabicyclo[3.1.0]hexane-6-carboxylate (P9, 0.1 g) in 0.5 mL of dry THF, BH₃-THF complex in THF (1M, 2.6 mL) was slowly added at 0° C. under N₂. The reaction mixture was refluxed for 6 h then cooled to 0° C. and first methanol (1 mL) and than HCl (1M in ether, 5 mL) were added cautiously and the reaction mixture stirred for 2 h. The solvent was partially removed under vacuum and the residue was loaded on SCX column eluting with NH₃/MeOH (2M). The methanolic phase was evaporated under vacuum and the crude affording the title product compound (70 mg).

MS (m/z): 239 [MH]⁺.

### Preparation 11: trifluoromethyl(1R,5R,6R/1S,5S,6S)-1-(3,4-dichlorophenyl)-6-(hydroxymethyl)-3-azabicyclo[3.1.0]hexane-3-carboxylate (P11)

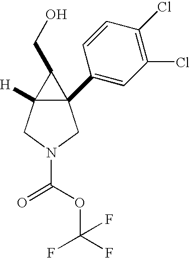

To a stirred solution of (1S,5S,6S/1R,5R,6R)-1-(3,4-dichlorophenyl)-3-azabicyclo[3.1.0]hex-6-yl]methanol (obtained following an analogous procedure to that described to obtain E1, 500 mg) in dichloromethane (19 mL) at room temperature, triethylamine (0.33 mL) and trifluoroacetic anhydride (302 μL) were added. Stirring was continued for 12 h; the reaction mixture was then diluted with dichloromethane and quenched with a saturated solution of NH₄Cl. The organic phase was dried and the solvent evaporated under vacuum. The crude product was purified by column chromatography (gradient cyclohexane/ethyl acetate from 90/10 to 70/30) to give the title compound (300 mg).

NMR (¹H, CDCl₃): δ ppm 7.38-7.51 (m, 2H), 7.15-7.23 (m, 1H), 4.22-4.42 (m, 1H), 3.91-4.09 (m, 1H), 3.66-3.81 (m, 1H), 3.30-3.64 (m, 3H), 1.92-2.05 (m, 1H), 1.36-1.43 (m, 1H), 1.24-1.32 (m, 1H).

### Preparation 12: 1,1-dimethylethyl(1R,5R,6R/1S,5S,6S)-1-(3,4-dichlorophenyl)-6-(hydroxymethyl)-3-azabicyclo[3.1.0]hexane-3-carboxylate (P12)

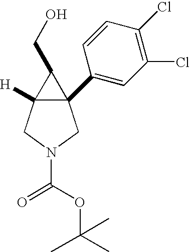

Method A:

To a stirred solution of (1S,5S,6S/1R,5R,6R)-1-(3,4-dichlorophenyl)-3-azabicyclo[3.1.0]hex-6-yl]methanol (obtained following an analogous procedure to that described to obtain E1, 500 mg) in dichloromethane (20 mL) at room temperature, triethylamine (0.4 mL) and bis(1,1-dimethylethyl)dicarbonate (455 mg) were added. Stirring was continued for 6 h then the reaction mixture was diluted with dichloromethane and quenched with a saturated solution of NH₄Cl. The organic phase was dried and the solvent evaporated under vacuum. The crude product was purified by column chromatography on silica (gradient cyclohexane/ethyl acetate from 80/20 to 10/10) to give the desired product (658 mg).

NMR (¹H, CDCl₃): δ ppm 7.33-7.45 (m, 2H), 7.10-7.21 (m, 2H), 3.94-4.13 (m, 10.48 Hz, 1H), 3.69-3.87 (m, 1H), 3.26-3.65 (m, 4H), 1.78-1.87 (m, 1H), 1.51-1.64 (m, 9H), 1.38-1.45 (m, 1H), 1.17 (s, 1H).

Method B:

To a solution of ethyl(1S,5S,6S/1R,5R,6R or 1S,5S,6R/1R,5R,6S )-1-(3,4-dichlorophenyl)-2,4-dioxo-3-azabicyclo[3.1.0]hexane-6-carboxylate (P6, 7.44 g) in tetrahydrofuran (20 mL) was slowly added BH3-THF complex (sol 1M in THF, 180 mL). The pale yellow solution was heated to gentle reflux and stirred at this temperature for 8 hrs, then stirred at room temperature overnight. The reaction mixture was cooled to 0° C., then MeOH (30 mL) and HCl (6N, 20 mL) were added dropwise. The mixture was stirred at room temperature for 30 min. NaOH (3N) was added until pH=10 (˜50 ml). A solution of BOC-Anhydride (5.26 mL) in THF (20 mL) was added and the mixture stirred at room temperature for 2 hrs. Et₂O (500 mL) was added, the organic phase separated, then the aq layer back extracted with Et₂O (350 mL). The combined organic layers were dried over Na2SO4, filtered and concentrated. 9.1 g of a colourless oil were recovered. It was purified by flash chromatography (eluent cyclohexane/ethyl acetate from 85:15 to 60:40). The title compound was obtained as white foam (4.66 g).

NMR (¹H, DMSO-d6): δ ppm 7.56-7.61 (1H, m), 7.52 (1H, d), 7.30 (1H, dd), 4.41 (1H, t), 3.89 (1H, t), 3.53 (1H, d), 3.37-3.48 (1H, m), 3.09-3.21 (2H, m), 2.99-3.11 (1H, m), 1.90-1.95 (1H, m), 1.37 (9H, s), 1.01-1.11 (1H, m); MS (m/z): 343 [MH-CH3]⁺.

### Preparation 13: 1,1-dimethylethyl(1R,5R,6R/1S,5S,6S)-6-(bromomethyl)-1-(3,4-dichlorophenyl)-3-azabicyclo[3.1.0]hexane-3-carboxylate (P13)

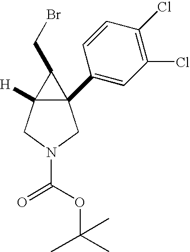

To a solution of 1,1-dimethylethyl(1R,5R,6R/1S,5S,6S)-1-(3,4-dichlorophenyl)-6-(hydroxymethyl)-3-azabicyclo[3.1.0]hexane-3-carboxylate (P12, 170 mg) in CH₂Cl₂ (5 mL), at 0° C., TEA (0.073 mL) and methansulfonyl chloride (0.052 mL) were added. The reaction mixture was stirred at room temperature overnight and then it was quenched with NH₄Cl sat (aq) and diluted with CH₂Cl₂. The organic layer was dried and concentrated in vacuo. The crude material 1,1-dimethylethyl(1R,5R,6R/1S,5S,6S)-1-(3,4-dichlorophenyl)-6-{[(methylsulfonyl)oxy]methyl}-3-azabicyclo[3.1.0]hexane-3-carboxylate (207 mg) was taken forward without further purification.

To a stirred solution of Copper(I) bromide-dimethylsulfide complex (108 mg) in 3 mL of THF was added EtMgBr 3M in diethyl ether (0.380 mL) at −78° C. After 30 min, at −78° C., 1,1-dimethylethyl(1R,5R,6R/1S,5S,6S)-1-(3,4-dichlorophenyl)-6-{[(methylsulfonyl)oxy]methyl}-3-azabicyclo[3.1.0]hexane-3-carboxylate previously obtained (207 mg) in THF (2 mL) was added and the mixture was gradually warmed to room temperature. It was stirred for 3 h before quenching it with aqueous NH₄Cl saturated solution. The mixture was filtered thought a pad of Celite and the filtrate washed with NH₄OH and extracted with EtOAc. The extract was dried and evaporated in vacuo. The residue was purified by chromatography on silica (gradient cyclohexane/EtOAc from 90/10 to 80/20) affording 150 mg of the title compound.

MS (m/z): 364 [MH-56]+.

### Preparation 14: 1,1-dimethylethyl(1R,5R,6R/1S,5S,6S)-1-(3,4-dichlorophenyl)-6-{[(methylsulfonyl)oxy]methyl}-3-azabicyclo[3.1.0]hexane-3-carboxylate (P14)

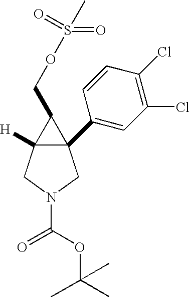

To a solution of 1,1-dimethylethyl(1R,5R,6R/1S,5S,6S)-1-(3,4-dichlorophenyl)-6-(hydroxymethyl)-3-azabicyclo[3.1.0]hexane-3-carboxylate (P12, 300 mg) in DCM (4 mL), at 0° C., TEA (0.128 mL) and methansulphonyl chloride (0.091 mL) were added. The reaction mixture was stirred at room temperature overnight and then it was quenched with NH₄Cl sat (aq) and diluted with CH₂Cl₂. The organic layer was dried and concentrated in vacuo to give the title compound (308 mg).

Rt=0.87 min.

UPLC conditions: Acquity™ UPLC system coupled with a ZQ single quadrupole mass

spectrometer (Waters-Micromass); operated in positive electrospray ionisation (ES+) mode. Column Acquity UPLC™ BEH C18, 1.7 μm 2.1×50 mm; Column temperature 40 C; Mobile phase: A: H2O+0.1% HCOOH; B: CH3CN+0.06% HCOOH

Gradient: t=0 min 3% (B); t=0.05 min 6% (B); t=0.57 min 70% (B); t=1.06 min 99% (B);t=1.44 min 99% (B); t=1.45 min 3% (B); Stop time: 1.50 min.

Flow rate: 1 ml/min; UV range: 210-350 nm; Sampling Rate: 20 points/sec, ZQ/MS

Ionization: ES+; Mass range: 100-1000 amu; Scan/interscan: 0.10 sec/0.01 sec; Polarity mode interscan: 0.025 sec; DAD/MS data offset time: +0.01 min.

### Preparation 15: 1,1-dimethylethyl(1S,5S,6S/1R,5R,6R)-1-(3,4-dichlorophenyl)-6-{[(4-fluorophenyl)oxy]methyl}-3-azabicyclo[3.1.0]hexane-3-carboxylate (P15)

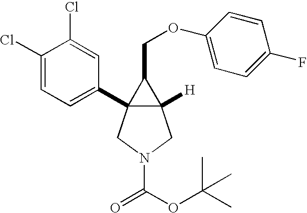

To a solution of 1,1-dimethylethyl(1S,5S,6S/1R,5R,6R)-1-(3,4-dichlorophenyl)-6-(hydroxymethyl)-3-azabicyclo[3.1.0]hexane-3-carboxylate (100 mg, prepared in a similar manner to that described for P12, but using dry THF instead of dichloromethane) in dry dichloromethane (2.8 mL), triethylamine (0.058 mL) and methanesulfonyl chloride (0.024 mL) were added at 0° C. After 5 min the reaction mixture was allowed to warm to room temperature. After 1 h water was added and the mixture was extracted with dichloromethane. The organic phase was washed with an aqueous saturated NH₄Cl solution, dried over Na₂SO₄ and concentrated in vacuo. The crude 1,1-dimethylethyl (1R,5R,6R/1S,5S,6S)-1-(3,4-dichlorophenyl)-6-{[(methylsulfonyl)oxy]methyl}-3-azabicyclo[3.1.0]hexane-3-carboxylate (160 mg) thus obtained was used without further purification.

Sodium hydride (60% dispersion in mineral oil, 34.1 mg) was added to a solution of 4-fluorophenol (92 mg) in dry DMF (1.4 mL) at 0° C. After 20 min the suspension was allowed to warm to room temperature and stirred for an additional 10 min. A solution of 1,1-dimethylethyl(1R,5R,6R/1S,5S,6S)-1-(3,4-dichlorophenyl)-6-{[(methylsulfonyl)oxy]methyl}-3-azabicyclo[3.1.0]hexane-3-carboxylate above prepared (160 mg) in dry DMF (1.4 mL) was then added and the mixture was stirred at room temperature for 1 h, then heated at 220° C. for 1 h 30 min. An aqueous saturated NH₄Cl solution was then added and the mixture was extracted with dichloromethane. The organic phase was washed with brine, dried over Na₂SO₄ and concentrated in vacuo. The residue was purified by flash chromatography (Biotage Si 25S column, gradient cyclohexane/ethyl acetate from 93/7 to 40/60 in 10 CV), to give 185 mg of a pale yellow oil. This was dissolved in dichloromethane and the solution thus obtained was washed with an aqueous 1M NaOH solution and water, dried over Na₂SO₄ and concentrated in vacuo. The compound thus obtained (127 mg) was further purified by a silica cartridge (5 g, gradient cyclohexane/ethyl acetate from 95/5 to 90/10) to give 123 mg of the title compound as pale yellow oil.

MS (m/z): 396 [MH-56]+.

### Preparation 16: 1,1-dimethylethyl(1S,5S,6S/1R,5R,6R)-1-(3,4-dichlorophenyl)-6-formyl-3-azabicyclo[3.1.0]hexane-3-carboxylate (P16)

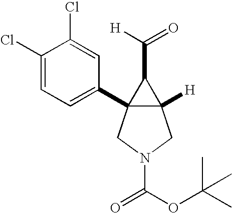

Dess-Martin periodinane (123 mg) was added at 25° C. to a solution of 1,1-dimethylethyl (1R,5R,6R/1S,5S,6S)-1-(3,4-dichlorophenyl)-6-(hydroxymethyl)-3-azabicyclo[3.1.0]hexane-3-carboxylate (P12, 80 mg) in dry dichloromethane (2 mL). After 30 min sodium thiosulfate (280 mg) and a saturated NaHCO₃ aqueous solution (15 mL) were added, and the mixture was stirred at room temperature for 30 min. The mixture was then extracted with dichloromethane; the organic phase was washed with brine, dried over Na₂SO₄ and concentrated in vacuo to give 83 mg of the title compound as a colourless oil.

NMR (¹, CDCl₃): δ ppm 9.07 (bs, 1H), 7.42 (m, 2H), 7.12 (m, 1H), 4.20-3.62 (m, 3H), 3.40 (s, 1H), 2.68 (m, 1H), 2.20 (s, 1H), 1.55 (s, 9H). MS (m/z): 300 [MH-56]+.

### Preparation 17: 1,1-dimethylethyl(1R,5S,6S/1R,5R,6R)-1-(3,4-dichlorophenyl)-6-[(dimethylamino)methyl]-3-azabicyclo[3.1.0]hexane-3-carboxylate (P17)

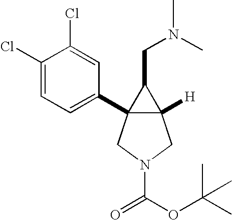

To a solution of 1,1-dimethylethyl(1S,5S,6S/1R,5R,6R)-1-(3,4-dichlorophenyl)-6-formyl-3-azabicyclo[3.1.0]hexane-3-carboxylate (P16, 83 mg) in dry tetrahydrofuran (2.3 mL) dimethylamine (0.349 mL), acetic acid (0.043 mL) and sodium triacetoxyborohydride (173 mg) were added at 25° C. After 1 h stirring at room temperature, the mixture was concentrated in vacuo Dichloromethane was then added and the organic phase was washed with an aqueous saturated NaHCO₃ solution and brine, dried over Na₂SO₄ and concentrated in vacuo. The crude material was purified by flash chromatography (Biotage Si 12S column, gradient cyclohexane/ethyl acetate from 90/10 to 0/100 and then ethyl acetate/methanol 100/1) to give 35 mg of the the title compound as a colourless oil.

NMR (¹H, CDCl₃): δ ppm 7.40-7.30 (m, 2H), 7.09 (m, 1H), 4.05-3.68 (m, 2H), 3.55 (m, 1H), 3.35 (m, 1H), 2.32 (m, 1H), 2.19 (s, 6H), 1.80 (m, 1H), 1.65 (m, 1H), 1.45 (s, 9H), 1.20 (m, 1H). MS (m/z): 385 [MH]+.

### Preparation 18: 1,1-dimethylethyl(1S,5S,6S/1R,5R,6R)-1-(3,4-dichlorophenyl)-6-[(methylthio)methyl]-3-azabicyclo[3.1.0]hexane-3-carboxylate (P18)

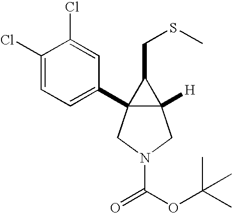

Triethylamine (0.047 mL) and methanesulfonyl chloride (0.019 mL) were added at 0° C. to a solution of 1,1-dimethylethyl(1R,5R,6R/1S,5S,6S)-1-(3,4-dichlorophenyl)-6-(hydroxymethyl)-3-azabicyclo[3.1.0]hexane-3-carboxylate (P12, 80 mg) in dry dichloromethane (2.2 mL). The reaction mixture was stirred at 25° C. for 1 h, then water was added and the mixture was extracted with dichloromethane. The organic phase was washed with an aqueous saturated NH₄Cl solution, dried over Na₂SO₄ and concentrated in vacuo to give 103 mg of crude 1,1-dimethylethyl(1R,5R,6R/1S,5S,6S)-1-(3,4-dichlorophenyl)-6-{[(methylsulfonyl)oxy]methyl}-3-azabicyclo[3.1.0]hexane-3-carboxylate, which was used without further purification. It was dissolved in dry N,N-dimethylformamide (2.5 mL) and sodium thiomethoxide (47.2 mg) was added. The mixture was stirred overnight at room temperature. An aqueous saturated NaHCO₃ solution was then added and the mixture was extracted with dichloromethane. The organic phase was washed with brine, dried over Na₂SO₄ and concentrated in vacuo. The residue was purified by flash chromatography (Biotage Si 25S column, gradient cyclohexane/ethyl acetate from 98/2 to 80/20) to give 21 mg of the title compound.

NMR (¹, CDCl₃): δ ppm 7.42-7.22 (m, 2H), 7.09 (m, 1H), 4.05-3.65 (m, 2H), 3.55 (m, 1H), 3.35 (m, 1H), 2.50-2.30 (m, 1H), 2.19 (s, 3H), 2.19 (m, 1H), 1.80 (m, 1H), 1.45 (s, 9H), 1.20 (m, 1H). MS (m/z): 332 [MH-56]+.

### Preparation 19: 1-{[2,4-bis(methyloxy)phenyl]methyl}-3-(3,4-dichlorophenyl)-1H-pyrrole-2,5-dione (P19)

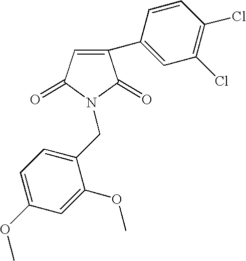

The title compound was prepared in 5.65 g yield from 1-{[2,4-bis(methyloxy)phenyl]methyl}-3-bromo-1H-pyrrole-2,5-dione (45 g, obtained following an analogous procedure to that described to obtain P7) following an analogous procedure to that described in Preparation 4 (P4).

NMR (¹H, CDCl₃) δ 8.1 (s, 1H), 7.8 (d, 1H), 7.5 (d, 1H), 7.17 (d, 1H), 6.8 (d, 1H), 6.45 (m, 2H), 4.82 (s, 2H), 3.85 (s, 3H), 3.80 (s, 3H).

### Preparation 20: ethyl(1S,5S,6S/1R,5R,6R )-3-{[2,4-bis(methyloxy)phenyl]methyl}-1-(3,4-dichlorophenyl)-2,4-dioxo-3-azabicyclo[3.1.0]hexane-6-carboxylate (P20)

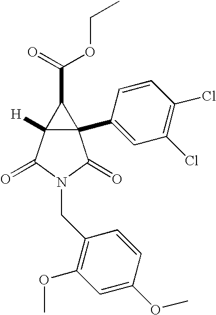

To a solution of 1-{[2,4-bis(methyloxy)phenyl]methyl}-3-(3,4-dichlorophenyl)-1H-pyrrole-2,5-dione (5.6 g, P19) in DCM ethyldiazoacetate was added (1.8 g). The mixture was stirred at room temperature for 24 h. One more equivalent of ethyldiazoacetate was added and the solution was stirred for other 24 h. After this time one further equivalent of ethyldiazoacetate was added and the solution was stirred for further 24 h. The mixture was diluted in DCM and washed with water. The organic phase was dried, filtered and the solvent evaporated under reduced pressure. The crude material thus recovered was triturated with ether, filtered and the solid obtained dried in vacuo to give ethyl 5-{[2,4-bis(methyloxy)phenyl]methyl}-6a-(3,4-dichlorophenyl)-4,6-dioxo-1,3a,4,5,6,6a-hexahydropyrrolo[3,4-c]pyrazole-3-carboxylate (5.28 g). The compound thus obtained (5.28 g) was heated at 200° C. for 36 h. The reaction mixture was then cooled down to room temperature and purify by flash chromatography (eluent cyclohexane/ethyl acetate=8:2) to give the title compound in 3.2 g yield

NMR (¹, CDCl₃) δ 7.55 (s, 1H), 7.45 (d, 1H), 7.25 (d, 1H), 7.17 (d, 1H), 6.45 (m, 2H), 4.66-4.46 (dd, 2H), 3.98 (q, 2H), 3.82 (s, 3H), 3.81 (s, 3H), 3.39 (d, 1H), 2.61 (d, 1H), 1.06 (t, 3H).

### Preparation 21: (1S,5S,6S/1R,5R,6R )-3-{[2,4-bis(methyloxy)phenyl]methyl}-1-(3,4-dichlorophenyl)-2,4-dioxo-3-azabicyclo[3.1.0]hexane-6-carboxylic acid (P21)

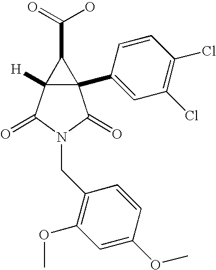

To a solution of ethyl(1S,5S,6S/1R,5R,6R )-3-{[2,4-bis(methyloxy)phenyl]methyl}-1-(3,4-dichlorophenyl)-2,4-dioxo-3-azabicyclo[3.1.0]hexane-6-carboxylate (P20, 1.0 g) in acetic acid (5 mL) 6N HCl (1 mL) was added and the mixture was heated to 90° C. under stirring for 24 h. The reaction mixture was evaporated under reduced pressure to give 0.9 g of the title compound

NMR (¹, CDCl₃) δ 7.57 (s, 1H), 7.46 (d, 1H), 7.26 (d, 1H), 7.17 (d, 1H), 6.46 (m, 2H), 4.66-4.47 (dd, 2H), 3.81 (s, 6H), 3.38 (d, 1H), 2.62 (d, 1H).

### Preparation 22: (1R,2S,5R,6R/1S,2R,5S,6S) or (1R,2R,5R,6R/1S,2S,5S,6S)-3-{[2,4-bis(methyloxy)phenyl]methyl}-1-(3,4-dichlorophenyl)-2-hydroxy-2-methyl-4-oxo-3-azabicyclo[3.1.0]hexane-6-carboxylic acid (P22)

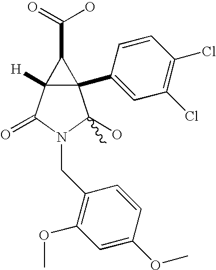

To a solution of (1S,5S,6S/1R,5R,6R)-3-{[2,4-bis(methyloxy)phenyl]methyl}-1-(3,4-dichlorophenyl)-2,4-dioxo-3-azabicyclo[3.1.0]hexane-6-carboxylic acid (P21, 300 mg) in THF (6.5 mL) a solution of methylmagnesium iodide in diethyl ether (3M, 0.44 mL) was added dropwise at −78° C. under a N₂atmosphere. The mixture was stirred 2 h at −78° C. then it was allowed to warm to room temperature and stirred overnight. The mixture was cooled down to 0° C. and methylmagnesium iodide in diethyl ether (3M, 0.44 mL) was added dropwise and the mixture stirred 3 h at rt. The mixture was cooled down to 0° C., HCl 0.5M was added dropwise and the product extracted with DCM. The organic phase was dried over Na₂SO₄, filtered and the solvent evaporated under reduced pressure. The crude material thus obtained was purified by flash chromatography on silica gel (eluent DCM: [DCM:MeOH:AcOH=9:1:0.1]=9:1) to give 205 mg of the title compound.

NMR (¹H, DMSO-d₆) δ 7.85 (s, 1H), 7.68 (d, 1H), 7.55 (d, 1H), 7.02 (d, 1H), 6.54 (s, 1H), 6.44 (d, 1H), 4.30 (d, 2H), 3.77 (s, 3H), 3.73 (s, 3H), 3.57 (d, 1H), 3.14 (d, 1H).

### Preparations 23 and 24: [(1S,2S,5S,6S/1R,2R,5R,6R) or (1S,2R,5S,6S/1R,2S,5R,6R)-3-([2,4-bis(methyloxy)phenyl]methyl)-1-(3,4-dichlorophenyl)-2-methyl-3-azabicyclo[3.1.0]hex-6-yl]methanol (P23) and [(1S,2S,5S,6S/1R,2R,5R,6R ) or (1S,2R,5S,6S/1R,2S,5R,6R )-3-{[2,4-bis(methyloxy)phenyl]methyl}-1-(3,4-dichlorophenyl)-2-methyl-3-azabicyclo[3.1.0]hex-6-yl]methanol (P24)

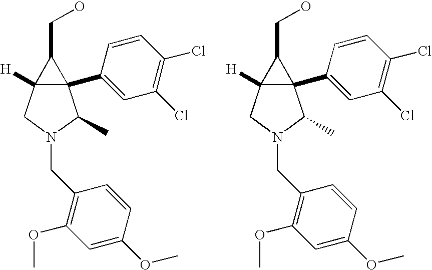

To a solution of (1R,2S,5R,6R/1S,2R,5S,6S) or (1R,2R,5R,6R/1S,2S,5S,6S)-3-{[2,4-bis(methyloxy)phenyl]methyl}-1-(3,4-dichlorophenyl)-2-hydroxy-2-methyl-4-oxo-3-azabicyclo[3.1.0]hexane-6-carboxylic acid (205 mg, P22) in THF (2 mL) at 0° C. BH₃-THF complex in THF (2.64 mL, 1M) was added and the mixture was stirred for 12 h at reflux temperature. The mixture was cooled down to 0° C., and additional BH₃-THF complex in THF (1.5 eq, 1M) was added to the reaction mixture. It was stirred at reflux temperature for 1 h and then cooled down to 0° C. Methanol (1 mL) first and then HCl (5 mL, 1M in Et2O) were added dropwise and the mixture was stirred at 40° C. for 1 h. The solvent was evaporated under reduced pressure and the crude loaded on SCX cartridge, washed with methanol and eluted with ammonia in methanol (1M). The solid recovered after evaporation in vacuo of the solvent was purified by flash chromatography on silica column (eluent DCM:[DCM/MeOH/NH3acq=90/10/0.5] gradient of DCM from 100 to 20%) to give [(1S,2S,5S,6S/1R,2R,5R,6R) or (1S,2R,5S,6S/1R,2S,5R,6R)-3-{[2,4-bis(methyloxy)phenyl]methyl}-1-(3,4-dichlorophenyl)-2-methyl-3-azabicyclo[3.1.0]hex-6-yl]methanol (35 mg) as pale yellow oil (P23).

NMR (¹, CDCl₃) δ 7.37 (m, 2H), 7.26 (d, 1H), 7.14 (d, 1H), 6.51-6.46 (m, 2H), 3.83-3.80 (m, 7H), 3.72 (d, 1H), 3.59 (d, 1H), 3.38 (m, 3H), 3.19 (d, 1H), 2.66 (d, 1H), 2.06 (m, 1H), 1.52 (d, 1H), 1.10 (d, 3H).

and [(1S,2R,5S,6S/1R,2S,5R,6R) or [(1S,2S,5S,6S/1R,2R,5R,6R) -3-{[2,4-bis(methyloxy)phenyl]methyl}-1-(3,4-dichlorophenyl)-2-methyl-3-azabicyclo[3.1.0]hex-6-yl]methanol (35 mg) as pale yellow oil (P24).

NMR (¹, CDCl₃) δ 7.34 (m, 2H), 7.17 (d, 1H), 7.09 (d, 1H), 6.45 (m, 2H), 3.83-3.78 (m, 8H), 3.40 (m, 2H), 3.32 (m, 2H), 2.93 (m, 1H), 2.39 (d, 1H), 2.02 (m, 1H), 1.68 (m, 1H), 1.23 (d, 3H).

### Preparation 25: [(1S,2R,5S,6S/1R,2S,5R,6R ) or (1S,2S,5S,6S/1R,2R,5R,6R)-3-{[2,4-bis(methyloxy)phenyl]methyl}-1-(3,4-dichlorophenyl)-6-[(ethyloxy)methyl]-2-methyl-3-azabicyclo[3.1.0]hexane (P25)

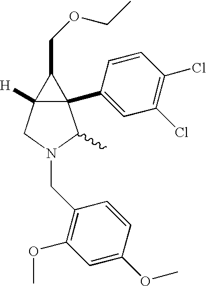

To a solution of [[(1S,2R,5S,6S/1R,2S,5R,6R ) or (1S,2S,5S,6S/1R,2R,5R,6R )-3-{[2,4-bis(methyloxy)phenyl]methyl}-1-(3,4-dichlorophenyl)-2-methyl-3-azabicyclo[3.1.0]hex-6-yl]methanol (35 mg, P23) in dry dichloromethane (1 mL) methansulfonyl chloride (7.1 μl) was added at 0° C. After 10 min the reaction was warmed at room temperature and stirred overnight. Water was added to the reaction mixture and the product was extracted with dichloromethane. The organic phase was dried over Na₂SO₄ and concentrated in vacuo to give [(1S,2R,5S,6S/1R,2S,5R,6R) or (1S,2S,5S,6S/1R,2R,5R,6R)-3-{[2,4-bis(methyloxy)phenyl]methyl}-1-(3,4-dichlorophenyl)-2-methyl-3-azabicyclo[3.1.0]hex-6-yl]methyl methanesulfonate as crude product.

To a suspension of sodium hydride in DMF (0.5 mL) ethanol (14 μL) was added at 0° C. After 30 min at 0° C. the suspension was allowed to warm to rt and it was stirred for 30 min. A solution of [(1S,2R,5S,6S/1R,2S,5R,6R) or (1S,2S,5S,6S/1R,2R,5R,6R)-3-{[2,4-bis(methyloxy)phenyl]methyl}-1-(3,4-dichlorophenyl)-2-methyl-3-azabicyclo[3.1.0]hex-6-yl]methyl methanesulfonate (39 mg, obtained as described above) in DMF (0.5 mL) was added and the mixture was stirred at 60° C. for 6 h and then at rt overnight. It was cooled down to 0° C. and a solution of sodium ethoxide in ethanol (21 % w, 50 μL) was added. The mixture was stirred at 60° C. for 4 h. The mixture was cooled down to rt, chilled water was added and the product extracted with EtOAc. The organic phase was washed with brine, dried over sodium sulfate and concentrated in vacuo. The crude material was purified by flash chromatography on silica column (eluent: EtOAc/cyclohexane=3:7) to give the title compound (15 mg).

NMR (¹, CDCl₃) δ 7.38 (m, 2H), 7.25 (d, 1H), 7.14 (d, 1H), 6.51-6.46 (m, 2H), 3.83 (s, 3H), 3.80 (s, 3H), 3.72 (d, 1H), 3.54 (d, 1H), 3.40 (m, 1H), 3.32-3.17 (m, 5H), 2.66 (d, 1H), 2.20 (m, 1H), 1.55 (d, 1H), 1.28 (m, 3H), 1.09 (t, 3H).

### Preparation 26: [(1S,2R,5S,6S/1R,2S,5R,6R) or (1S,2S,5S,6S/1R,2R,5R,6R)-3-{[2,4-bis(methyloxy)phenyl]methyl}-1-(3,4-dichlorophenyl)-6-[(ethyloxy)methyl]-2-methyl-3-azabicyclo[3.1.0]hexane (P26)

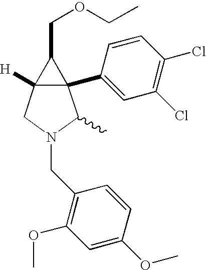

The title compound was prepared as yellow oil in 63 mg yield from [(1S,2R,5S,6S/1R,2S,5R,6R) or (1S,2S,5S,6S/1R,2R,5R,6R)-3-{[2,4-bis(methyloxy)phenyl]methyl}-1-(3,4-dichlorophenyl)-2-methyl-3-azabicyclo[3.1.0]hex-6-yl]methanol (100 mg, prepared with an analogous method to that described for Preparation 24) following an analogous procedure to that described for Preparation 25.

NMR (¹, CDCl₃) δ 7.40 (s, 1H), 7.31 (d, 1H), 7.19 (s, 1H), 7.12 (s, 1H), 6.46-6.43 (m, 2H), 3.81 (s, 3H), 3.77 (s, 3H), 3.37-3.19 (m, 5H), 2.95-2.89 (m, 2H), 2.36 (d, 1H), 2.00 (m, 1H), 1.59 (m, 1H), 1.52 (m, 1H), 1.23 (d, 3H), 1.11 (t, 3H).

### Preparation 27: 1,1-dimethylethyl-(1R,5S,6S/1S,5R,6R)-1-(3,4-dichlorophenyl)-6-ethenyl-3-azabicyclo[3.1.0]hexane-3-carboxylate (P27)

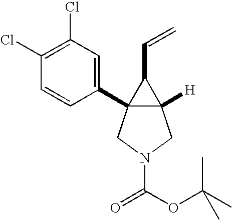

To a stirred solution of 1,1-dimethylethyl(1S,5S,6S/1R,5R,6R)-1-(3,4-dichlorophenyl)-6-(hydroxymethyl)-3-azabicyclo[3.1.0]hexane-3-carboxylate (P12 Method B, 0.5 g) in DCM (15 mL), at 0° C., Dess-Martin periodinane (0.710 g) was added portionwise then the reaction was allowed to reach room temperature. After 2 h an additional amount of Dess-Martin reagent was added (0.1 g) and the reaction was further stirred for 1 h. Aqueous concentrated NaHCO₃ solution (8 mL) and a sodium thiosulfate solution (2 g in 5 mL water) were added, the mixture was stirred for 1 h. The mixture was extracted with DCM; the organic phase was dried over sodium sulphate and evaporated under reduced pressure to give 0.48 g of the corresponding crude 1,1-dimethylethyl (1S,5S,6S/1R,5R,6R)-1-(3,4-dichlorophenyl)-6-formyl-3-azabicyclo[3.1.0]hexane-3-carboxylate as a white foam.

To a stirred suspension of methyl(triphenyl)phosphonium bromide (0.626 g) in THF (3 mL) at 0° C., butyllithium (0.70 mL, 2.5M in hexane) was added dropwise. The dark yellow reaction mixture was allowed to reach room temperature and stirred for 20 min, then cooled to 0° C. and the above described crude 1,1-dimethylethyl(1S,5S,6S/1R,5R,6R)-1-(3,4-dichlorophenyl)-6-formyl-3-azabicyclo[3.1.0]hexane-3-carboxylate (0.48 g) in THF (1.5 mL) was added dropwise. The ice-bath was removed and the reaction mixture stirred for 6 h at room temperature. Diethyl ether and water were added, the organic phase was dried on sodium sulphate, the solvent evaporated under vacuum and the crude product was purified by FC (eluting with cyclohexane/ethyl acetate from 1/0 to 9/1) to give 0.277 g of the title compound as an oil.

1H NMR (¹, CDCl₃) δ ppm 7.36-7.43 (m, 1H) 7.30-7.35 (m, 1H) 7.02-7.12 (m, 1H) 4.98-5.18 (m, 2H) 4.88-4.96 (m, 1H) 3.88-4.09 (m, 1H) 3.72-3.87 (m, 1H) 3.53-3.65 (m, 1H) 3.37 (d, 1H) 1.89-1.96 (m, 1H) 1.72-1.81 (m, 1H) 1.43-1.51 (m, 9H); (MS (m/z): 298 [MH —C₄H₈]⁺).

### Preparation 28: ethyl 2-diazopropanoate (P28)

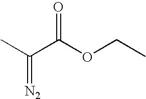

To a stirred solution of Tosyl-Cl (4 g) in acetone (60 mL), at 0° C., a solution of NaN₃ (1.37 g) in water (60.0 mL) was added and the reaction mixture was stirred at 0° C. for 2 h. Acetone was evaporated under reduced pressure and the aqueous phase was extracted twice with diethyl ether. The organic phase was dried over sodium sulphate and the solvent evaporated under vacuum to give 4 g of the crude tosyl azide intermediate as colourless oil. To a stirred suspension of NaH (0.810 g, 60% in oil) in dry diethyl ether (15 mL), at room temperature, a solution of ethyl 2-methyl-3-oxobutanoate (1.5 g) in diethyl ether (3 mL) was added dropwise over a period of 5 min. The reaction mixture was cooled to 0° C. and 1.1 g of the above tosyl azide was added dropwise over 5 mins. The reaction mixture was diluted with diethyl ether (10 mL), stirred for 45 min and the precipitate filtered off. The filtrate was extracted with water (100 mL), the organic phase dried over sodium sulphate and the solvent removed under reduced pressure to give 0.16 g of the crude title compound.

¹H NMR (400 MHz, CHLOROFORM-d) δ ppm: 4.23 (q, 2H) 1.92-2.01 (s, 3H) 1.23-1.34 (t, 3H).

### Preparation 29: 1,1-dimethylethyl-(1R,5R,6R/1S,5S,6S)-1-(3,4-dichlorophenyl)-6-(hydroxymethyl)-6-methyl-3-azabicyclo[3.1.0]hexane-3-carboxylate and 1,1-dimethylethyl-(1R,5R,6S/1S,5S,6R)-1-(3,4-dichlorophenyl)-6-(hydroxymethyl)-6-methyl-3-azabicyclo[3.1.0]hexane-3-carboxylate (P29)

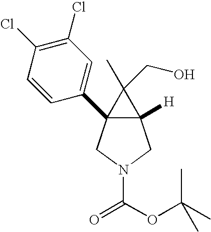

A solution of 3-(3,4-dichlorophenyl)-1H-pyrrole-2,5-dione (obtained following an analogous procedure to that described to obtain P1, 0.1 g) and ethyl 2-diazopropanoate (P28, 0.16 g) in toluene (2.2 mL) was warmed to 100° C. and stirred for 3 h. After this period of time the solvent was removed under reduced pressure and the crude product was dissolved in THF (4 mL), BH₃THF complex (3.80 mL, 1M in THF) was added dropwise and the resulting reaction mixture was refluxed for 4 h. The reaction was cooled to 0° C., 2N HCl was added and the mixture was stirred for 0.5 h at room temperature. The pH of the mixture was taken to ˜9 with saturated sodium carbonate and the reaction mixture was extracted with DCM. The organic phase was washed with brine, dried over sodium sulphate and the solvent evaporated under reduced pressure to give the corresponding crude [(1R,5R/1S,5S)-1-(3,4-dichlorophenyl)-6-methyl-3-azabicyclo[3.1.0]hex-6-yl]methanol (MS(m/z): 272 [MH]⁺). This material was dissolved in DCM (4 mL), bis(1,1-dimethylethyl)dicarbonate (90 mg) was added and the mixture was stirred overnight. The reaction mixture was washed at first with saturated NaHCO₃ and then with brine. The organic phase was dried over sodium sulphate and the solvent was removed under reduced pressure to give 42 mg of the crude title product as a white foam.

MS(m/z): 315.97 [MH —C₄H₈]⁺.

### Preparation 30: 1,1-dimethylethyl(1R,5S,6S/1S,5R,6R)-1-(3,4-dichlorophenyl)-6-(2-hydroxyethyl)-3-azabicyclo[3.1.0]hexane-3-carboxylate (P30)

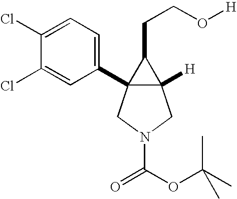

To a stirred solution of 1,1-dimethylethyl-(1R,5S,6S/1S,5R,6R)-1-(3,4-dichlorophenyl)-6-ethenyl-3-azabicyclo[3.1.0]hexane-3-carboxylate (P27) (0.138 g) in THF (2 mL) at 0° C. and under a nitrogen atmosphere, BH₃-THF complex (0.467 mL, 1M in THF) was added dropwise. The ice-bath was removed and the reaction mixture stirred for 3.5 h at room temperature. The mixture was cooled to 0° C. and quenched by adding water (0.6 mL). 3M NaOH (1.4 mL) and 30% H₂O₂ (1.4 ml) were then added and the resulting mixture stirred for 0.5 h. The reaction mixture was diluted with water and extracted with ethyl acetate; the organic phase was dried over sodium sulphate and the solvent removed under reduce pressure to give 77 mg of the title compound.

(MS(m/z): 316 [MH —C₄H₈]⁺).

### Examples 1 and 2 (Method A)

### [(1S,5S,6S/1R,5R,6R)-1-(3,4-dichlorophenyl)-3-azabicyclo[3.1.0]hex-6-yl]methanol (E1) and [(1S,5S,6R/1R,5R,6S)-1-(3,4-dichlorophenyl)-3-azabicyclo[3.1.0]hex-6-yl]methanol (E2)

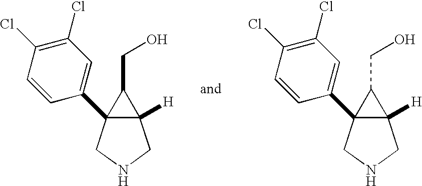

To a stirred solution of ethyl(1S,5S,6S/1R,5R,6R) and (1S,5S,6R/1R,5R,6S)-1-(3,4-dichlorophenyl)-2,4-dioxo-3-azabicyclo[3.1.0]hexane-6-carboxylate (P2, 0.78 g) in 1 mL of dry THF, BH₃-THF complex in THF (1M, 19 mL) was slowly added at 0° C. under N₂. The reaction mixture was refluxed for 6 h then cooled to 0° C. and aqueous HCl (6N, 7.5 mL) was added cautiously and the reaction mixture stirred for 1 h. The solvent was partially removed under vacuum and the residue was loaded on MCX (Mixed-mode strong Cation eXchange) column eluting with NH₃/MeOH (2M). The methanolic phase was evaporated under vacuum and the crude was purified by flash chromatography (eluting with dichloromethane/methanol/ammonia 33% 95:5:0.5) to give:

Example 1 (E1): [(1S,5S,6S/1R,5R,6R)-1-(3,4-dichlorophenyl)-3-azabicyclo[3.1.0]hex-6-yl]methanol (E1) as a White Oil (165 mg)

NMR (¹, CDCl₃): δ 7.40 (m, 2H), 7.38 (d, 1H), 7.14 (dd, 1H), 3.51 (dd, 1H), 3.41 (dd, 1H), 3.32 (d, 1H), 3.12 (m, 2H), 2.91 (d, 1H), 1.69 (m, 1H), 1.39 (m, 1H). MS (m/z): 258 [MH]⁺.

The relative configuration was assessed on the basis of Roesy data.

Example 2 (E2): [(1S,5S,6R/1R,5R,6S)-1-(3,4-dichlorophenyl)-3-azabicyclo[3.1.0]hex-6-yl]methanol (350 mg).

NMR (¹, CDCl₃): δ 7.35 (d, 1H), 7.25 (d, 1H), 7.01 (d, 1H), 4.90 (m, 2H), 3.58 (d, 1H), 3.36 (m, 2H), 3.31 (m, 1H), 1.95 (m, 1H), 1.42 (m, 1H). MS (m/z): 258 [MH]⁺.

The relative configuration was assessed on the basis of Roesy data.

### Example 1 (Method B)

### [(1S,5S,6S/1R,5R,6R)-1-(3,4-dichlorophenyl)-3-azabicyclo[3.1.0]hex-6-yl]methanol (E1)

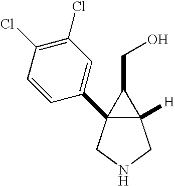

To a solution of ethyl(1S,5S,6S/1R,5R,6R or 1S,5S,6R/1R,5R,6S )-1-(3,4-dichlorophenyl)-2,4-dioxo-3-azabicyclo[3.1.0]hexane-6-carboxylate (P6, 380 mg) in THF (0.6 mL), at room temperature, was added BH₃-THF complex in THF (1M, 9 mL) and the mixture was refluxed for 6 h. MeOH (3 mL) and HCl 1M in Et₂O (15 mL) were then added and the solution was stirred at r.t. for 2 h. Once concentrated in vacuo, the crude was purified by SCX cartridge eluting with NH₃ 2M in MeOH to give the title compound (268 mg).

NMR (¹, CDCl₃): δ 7.40 (m, 2H), 7.16 (d, 1H), 3.54-3.34 (m, 3H), 3.16 (m, 2H), 2.92 (d, 1H), 1.71 (m, 1H), 1.42 (m, 1H). MS (m/z): 258 [MH]⁺.

The relative configuration was assessed on the basis of Roesy data.

### Example 3

### [(1S,5S,6R/1R,5R,6S)-1-(3,4-dichlorophenyl)-3-azabicyclo[3.1.0]hex-6-yl]methanol hydrochloride (E3)

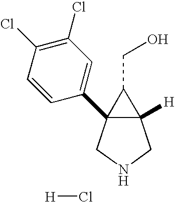

To a solution of [(1S,5S,6R/1R,5R,6S)-1-(3,4-dichlorophenyl)-3-azabicyclo[3.1.0]hex-6-yl]methanol (E2, 15 mg) in DCM (1 mL) was added HCl (55 μL, 1M in Et₂O), the solvent evaporated under vacuum and the material thus obtained triturated with Et₂O to give 15 mg of the title compound as a white slightly hygroscopic solid.

NMR (¹, DMSO-d6): δ 9.15 (bs, 1H), 8.85 (bs, 1H), 7.59 (d, 2H), 7.30 (d, 1H), 4.90 (bs, 1H), 3.69 (m, 2H), 3.62 (m, 2H), 3.25 (m, 2H), 2.45 (m, 1H), 1.65 (m, 1H). MS (m/z): 258 [MH]⁺.

### Example 4

### (1S,5S,6S/1R,5R,6R)-1-(3,4-dichlorophenyl)-6-[(methyloxy)methyl]-3-azabicyclo[3.1.0]hexane (E4)

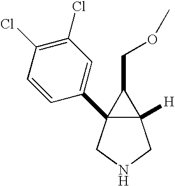

To a stirred solution of (1S,5S,6S/1R,5R,6R)-1-(3,4-dichlorophenyl)-3-azabicyclo[3.1.0]hex-6-yl]methanol (E1, 73 mg) in dichloromethane (3 mL) at room temperature, triethylamine (59 μL) and bis(1,1-dimethylethyl)dicarbonate (68 mg) were added. Stirring was continued for 6 h, then the reaction mixture was concentrated under vacuum and the crude product treated with dichloromethane and bicarbonate. The organic phase was dried over sodium sulfate and the solvent evaporated under vacuum to give a crude product. To a stirred solution of this crude material in dry DMF (3 mL) sodium hydride (18 mg) was added at 0° C. and the reaction mixture stirred at room temperature for 1 h. Methyl iodide (21 μL) was added and the reaction mixture was stirred at room temperature for 4 h. Ethyl acetate and water were added, the organic phase separated, washed with brine, dried over sodium sulfate and the solvent evaporated under vacuum to give a crude product.

To a solution of this crude in DCM (4 mL) TFA (1 ml) was added. The reaction mixture was stirred at room temperature for 1 h, it was concentrated in vacuo and the crude product was loaded on SCX column eluting with MeOH/NH₃ (2M). The crude material obtained was purify by flash chromatography (eluting with dichloromethane/methanol/ammonia acq. 95:5:0.5) to give 5 mg of the title compound as white oil.

NMR (¹, CDCl₃): δ 7.41 (d, 1H), 7.39 (d, 1H), 7.16 (dd, 1H), 3.30 (d, 1H), 3.21 (s, 3H), 3.20-3.10 (m, 4H), 3.90 (d, 1H), 1.74 (m, 1H), 1.37 (m, 1H). MS (m/z): 272 [MH]⁺.

An additional amount of Example 4 (95 mg), prepared with an analogous procedure, was submitted to semi-preparative HPLC to give the separated enantiomers, by using a chiral column chiralpak AD-H, eluent A: n-hexane; B: Ethanol, gradient isocratic 18% B, flow rate 14 mL/min, detection UV at 230 nm. Retention times given were obtained using an analytical HPLC using a chiral column chiralpak AD-H, 25×4.6 cm, eluent A: n-hexane; B: ethanol, gradient isocratic 20% B, flow rate 0.8 mL/min, detection UV at 230 nm.

Example 4A: (1S,5S,6S or 1R,5R,6R)-1-(3,4-dichlorophenyl)-6-[(methyloxy)methyl]-3-azabicyclo[3.1.0]hexane (Enantiomer 1) was recovered as a white solid (30 mg). Rt.=7.19 min.

Example 4B (1R,5R,6R or 1S,5S,6S)-1-(3,4-dichlorophenyl)-6-[(methyloxy)methyl]-3-azabicyclo[3.1.0]hexane (Enantiomer 2) was recovered as a white solid (30 mg). Rt.=8.54 min.

### Example 5

### (1S,5S,6S/1R,5R,6R)-1-(3,4-dichlorophenyl)-6-[(methyloxy)methyl]-3-azabicyclo[3.1.0]hexane hydrochloride (E5)

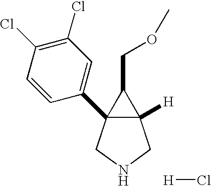

To a solution of (1S,5S,6S/1R,5R,6R)-1-(3,4-dichlorophenyl)-6-[(methyloxy)methyl]-3-azabicyclo[3.1.0]hexane (E4, 5 mg) in dichloromethane (1 mL) was added HCl (18 μL, 1M in Et₂O), the solvent evaporated under vacuum and the material thus obtained triturated with Et₂O to give 5 mg of the title compound as a white slightly hygroscopic solid.

NMR (¹, DMSO-d6): δ 9.30 (acidic proton), 7.72 (d, 1H), 7.61 (d, 1H), 7.39 (dd, 1H), 3.77 (d, 1H), 3.52 (dd, 1H), 3.40 (d, 1H), 3.19 (d, 1H), 3.06 (m, 1H), 3.04 (s, 3H), 2.97 (m, 1H), 2.24 (m, 1H), 1.69 (m, 1H). MS (m/z): 272 [MH]⁺.

### Example 6

### (1S,5S,6R/1R,5R,6S)-1-(3,4-dichlorophenyl)-6-[(methyloxy)methyl]-3-azabicyclo[3.1.0]hexane (E6)

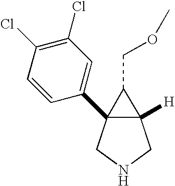

The title compound was prepared as white oil in 30 mg yield from (1S,5S,6R/1R,5R,6S)-1-(3,4-dichlorophenyl)-3-azabicyclo[3.1.0]hex-6-yl]methanol (E2, 80 mg) using a similar procedure as set out earlier in Example 4.

MS (m/z): 272 [MH]⁺.

### Example 7

### (1S,5S,6R/1R,5R,6S)-1-(3,4-dichlorophenyl)-6-[(methyloxy)methyl]-3-azabicyclo[3.1.0]hexane hydrochloride (E7)

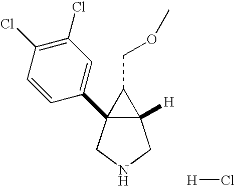

The title compound was prepared as white slightly hygroscopic solid in 30 mg yield from (1S,5S,6R/1R,5R,6S)-1-(3,4-dichlorophenyl)-6-[(methyloxy)methyl]-3-azabicyclo[3.1.0]hexane (E6, 30 mg) using a similar procedure as set out earlier in Example 5.

NMR (¹, DMSO-d6): δ 9.15 (acidic proton), 7.59 (d, 2H), 7.26 (d, 1H), 3.68-3.55 (m, 5H), 3.30 (s, 3H), 3.22 (m, 1H), 2.44 (m, 1H), 1.69 (m, 1H). MS (m/z): 272 [MH]⁺.

### Example 8

### (1S,5S,6S/1R,5R,6R)-6-{[(cyclopropylmethyl)oxy]methyl}-1-(3,4-dichlorophenyl)-3-azabicyclo[3.1.0]hexane (E8)

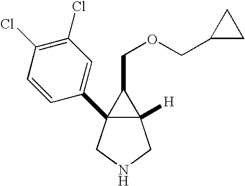

To a stirred solution of (1S,5S,6S/1R,5R,6R)-1-(3,4-dichlorophenyl)-3-azabicyclo[3.1.0]hex-6-yl]methanol (E1, 146 mg), (prepared following an analogous procedure to that described to obtain Example 1, method A), in DCM (5.5 mL) at room temperature, triethylamine (100 μL) and bis(1,1-dimethylethyl)dicarbonate (128.3 mg) were subsequently added. Stirring was continued for 2 h, then the reaction mixture was concentrated under vacuum and the crude product treated with dichloromethane and water. The organic phase was dried over sodium sulfate and the solvent evaporated under vacuum to give a crude product. To a stirred solution of this crude material (200 mg) in dry DMF (3 mL) sodium hydride (14 mg) was added at 0° C. and the reaction mixture stirred for 0.5 h, after which time bromomethylcyclopropane (56 μL) was added and the reaction mixture was stirred at room temperature for 4 h. Additional sodium hydride (14 mg) was added at 0° C. and the reaction mixture stirred for 1 h, then additional bromomethylcyclopropane (56 μL) was added and the reaction mixture was stirred at room temperature overnight. Ethyl acetate and chilly water were added, the organic phase separated, washed with brine, dried over sodium sulfate and the solvent evaporated under vacuum to give a crude product. To a solution of this crude (126 mg) in dichloromethane (5 mL), TFA (1 ml) was added at 0° C. The reaction mixture was stirred at reach room temperature for 1 h, it was concentrated in vacuo and the crude product was purified by flash chromatography (eluting with dichloromethane/methanol/33% aqueous ammonia 95:5:0.5) to give 62 mg of the title compound.

NMR (¹, CDCl₃): δ 7.45 (d, 1H), 7.37 (d, 1H), 7.20-7.17 (dd, 1H), 3.40-3.30 (m, 2H), 3.18-3.14 (m, 3H), 3.07-2.89 (m, 3H), 1.63-1.61 (m, 1H), 1.42-1.36 (m, 1H), 1.00-0.96 (m, 1H), 0.53-0.49 (m, 2H), 0.15-0.13 (m, 2H), NH not observed.

### Example 9

### (1S,5S,6S/1R,5R,6R)-6-{[(cyclopropylmethyl)oxy]methyl}-1-(3,4-dichlorophenyl)-3-azabicyclo[3.1.0]hexane hydrochloride (E9)

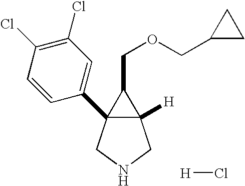

To a solution of (1S,5S,6S/1R,5R,6R)-6-{[(cyclopropylmethyl)oxy]methyl}-1-(3,4-dichlorophenyl)-3-azabicyclo[3.1.0]hexane (E8, 62 mg) in dichloromethane (1.5 mL) was added HCl (0.198 mL, 1M in Et₂O), the solvent evaporated under vacuum and the material thus obtained triturated with Et₂O to give 65 mg of the title compound.

NMR (¹, DMSO): δ 9.86-8.13 (br.s., 2H), 7.73.7.70 (d, 1H), 7.62-7.58 (d, 1H), 7.42-7.36 (dd, 1H), 3.81-3.70 (d, 1H), 3.52-3.45 (dd, 1H), 3.41-3.36 (d, 1H), 3.28-3.22 (dd, 1H), 3.20-3.14 (d, 1H), 3.09-3.02 (dd, 1H), 2.95-2.80 (m, 2H), 2.24-2.14 (m, 1H), 1.68-1.54 (m, 1H), 0.88-0.74 (m, 1H), 0.45-0.20 (m, 2H), -0.06-0.09 (m, 2H).

E9 (61 mg) was then submitted to semi-preparative HPLC to separate the racemic mixture into single enantiomers, by using a chiral column chiralpak AD-H, eluent A: n-hexane; B: Ethanol, gradient isocratic 20% B, flow rate 15 mL/min, detection UV at 225 nm. Retention times given were obtained using an analytical HPLC using a chiral column chiralpak AD-H, 25×4.6 cm, eluent A: n-hexane; B: ethanol, gradient isocratic 20% B, flow rate 0.8 mL/min, detection UV at 235 nm.

E8A: (1S,5S,6S or 1R,5R,6R)-6-{[(cyclopropylmethyl)oxy]methyl}-1-(3,4-dichlorophenyl)-3-azabicyclo[3.1.0] (Enantiomer 1) was recovered as a white solid, Rt.=6.04 min. (16 mg)

E8B: (1R,5R,6R or 1 S,5S,6S)-6-{[(cyclopropylmethyl)oxy]methyl}-1-(3,4-dichlorophenyl)-3-azabicyclo[3.1.0] (Enantiomer 2) was recovered as a white solid, Rt.=7.54 min. (16 mg)

### Example 10

### (1S,5S,6R/1R,5R,6S)-6-{[(cyclopropylmethyl)oxy]methyl}-1-(3,4-dichlorophenyl)-3-azabicyclo[3.1.0]hexane (E10)

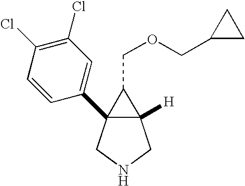

To a stirred solution of (1S,5S,6R/1R,5R,6S)-1-(3,4-dichlorophenyl)-3-azabicyclo[3.1.0]hex-6-yl]methanol (E2, 390 mg), (prepared following an analogous procedure to that described to obtain Example 1, method A), in DCM (15 mL) at room temperature, triethylamine (275 μL) and bis(1,1-dimethylethyl)dicarbonate (346 mg) were subsequently added. Stirring was continued for 2 h, then the reaction mixture was concentrated under vacuum and the crude product treated with dichloromethane and water. The organic phase was dried over sodium sulfate and the solvent evaporated under vacuum to give a crude product. To a stirred solution of this crude material (90 mg) in dry DMF (2.5 mL) sodium hydride (12 mg) was added at 0° C. and the reaction mixture stirred for 0.5 h then bromomethylcyclopropane (47 μL) was added and the reaction mixture was stirred at room temperature for 4 h. Additional sodium hydride (12 mg) was added at 0° C. and the reaction mixture stirred for 1 h, then bromomethylcyclopropane (47 μL) was added and the reaction mixture was stirred at room temperature overnight. Ethyl acetate and icy water were added, the organic phase separated, washed with brine, dried over sodium sulfate and the solvent evaporated under vacuum to give a crude product. To a solution of this crude (107 mg) in dichloromethane (5 mL) TFA (1 ml) was added at 0° C., the reaction mixture was stirred at room temperature for 1 h, it was concentrated in vacuo and the crude product was purified by flash chromatography (eluting with dichloromethane/methanol/33% aqueous ammonia 95:5:0.5) to give 16 mg of the Title compound.

NMR (¹, CDCl₃): δ 7.35 (d, 1H), 7.28 (s, 1H), 7.03-7.01(dd, 1H), 3.84-3.75 (m, 2H), 3.47-3.44 (d, 1H), 3.40-3.36 (dd, 1H), 3.34-3.31 (m, 3H), 3.26-3.23 (d, 1H), 1.97-1.93 (m, 1H), 1.54-1.49 (m, 1H), 1.12-1.07 (m, 1H), 0.58-0.54 (m, 2H), 0.24-0.21 (m, 2H), NH not detected.

### Example 11

### (1S,5S,6R/1R,5R,6S)-6-{[(cyclopropylmethyl)oxy]methyl}-1-(3,4-dichlorophenyl)-3-azabicyclo[3.1.0]hexane hydrochloride (E11)

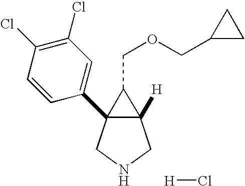

To a solution of (1S,5S,6R/1R,5R,6S)-6-[(cyclopropyloxy)methyl]-1-(3,4-dichlorophenyl)-3-azabicyclo[3.1.0]hexane (E10, 16 mg) in dichloromethane (0.5 mL) was added HCl (0.051 mL, 1M in Et₂O), the solvent evaporated under vacuum and the material thus obtained triturated with Et₂O to give 16 mg of the title compound.

NMR (¹, DMSO): δ 10.00-7.70 (br.s., 2H), 7.47-7.43 (d, 1H), 7.44-7.42 (d, 1H), 7.13-7.08 (dd, 1H), 3.57-3.49 (dd, 1H), 3.49-3.36 (m, 4H), 3.22-3.10 (m, 2H), 3.11-3.03 (d, 1H), 2.34-2.27 (m, 1H), 1.60-1.51 (m, 1H), 0.93-0.81 (m, 1H), 0.35-0.26 (m, 2H), −0.04-0.05 (m, 2H).

### Examples 12

### (1S,5S,6S/1R,5R,6R)-1-(3,4-dichlorophenyl)-6-[(ethyloxy)methyl]-3-azabicyclo[3.1.0]hexane (E12)

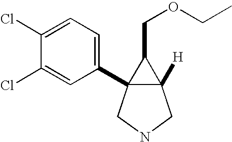

Method A: To a stirred solution of (1S,5S,6S/1R,5R,6R)-1-(3,4-dichlorophenyl)-3-azabicyclo[3.1.0]hex-6-yl]methanol (E1, 47.5 mg), (obtained following an analogous procedure to that described to obtain Example 1, method A), in dry DMF (1 mL), at 0° C., sodium hydride (7 mg, 60% in mineral oil) was added portionwise, followed after 0.5 h by ethyl iodide (16 μL). The reaction mixture was stirred for 4 hours, then water (2 mL) was added and the mixture extracted by ethyl ether (5 mL×2). The organic phase was dried over anhydrous sodium sulphate and evaporated under reduced pressure to give a crude product that was treated with a 3:1 mixture of DCM and trifluoroacetic acid (6:2 mL) at RT for 2 h. The pH of the reaction mixture was taken to ˜9 with sodium carbonate, the mixture concentrated under reduced pressure and the residue was extracted with DCM to give 16 mg of the title compound. MS (m/z): 286 [MH]⁺.

Method B: To a suspension of [(1S,5S,6S/1R,5R,6R)-1-(3,4-dichlorophenyl)-3-azabicyclo[3.1.0]hex-6-yl]methanol (E1, 610 mg) (riprepared following an analogous sequence as reported in Preparations 1, 2 and Example 1, Method A) and triethylamine (0.494 mL) in dry THF (15 mL), was added dropwise di-tert-butyl dicarbonate (567 mg) dissolved in THF (8 mL). The clear reaction mixture thus obtained was allowed to react at room temperature. After 2 h water was added and the mixture was extracted with ethyl acetate. The organic phase was washed with brine, dried over Na₂SO₄ and concentrated in vacuo. The crude product was purified by flash chromatography eluting with cyclohexane/ethyl acetate from 88/12 to 0/100 to give 815 mg of the corresponding N-Boc derivative. Another batch (356 mg) of this compound was prepared according to the same procedure reported above.

The mixture of the two batches (946 mg) was dissolved in dry dichloromethane (26 mL). Triethylamine (0.552 mL) and methanesulfonyl chloride (0.226 mL) were added at 0° C. After 10 min the reaction was warmed at room temperature and stirred overnight. Water was added and the mixture was extracted with dichloromethane. The organic phase was washed with an aqueous saturated NH₄Cl solution, dried over Na₂SO₄ and concentrated in vacuo, to give 1.17 g of crude 1,1-dimethylethyl(1S,5S,6S/1R,5R,6R)-1-(3,4-dichlorophenyl)-6-{[(methylsulfonyl)oxy]methyl}-3-azabicyclo[3.1.0]hexane-3-carboxylate, which was used without further purification.

Ethanol (0.470 mL) was added at 0° C. to a suspension of sodium hydride (0.332 g, 60% in mineral oil) in dry DMF (13 mL). After 30 min the suspension was allowed to warm at room temperature and stirred for an additional 30 min. A solution of 1,1-dimethylethyl (1S,5S,6S/1R,5R,6R)-1-(3,4-dichlorophenyl)-6-{[(methylsulfonyl)oxy]methyl}-3-azabicyclo[3.1.0]hexane-3-carboxylate (1.17 g) in dry DMF (13 mL) was then added and the mixture stirred overnight at 60° C. and at room temperature for another 48 h. An aqueous saturated NH₄Cl solution was added and the mixture was extracted with dichloromethane. The organic phase was washed with brine and chilled water, dried over Na₂SO₄ and concentrated in vacuo. The crude product was purified by flash chromatography (eluent ciclohexane/ethyl acetate from 95/5 to 60/40) to give 781 mg of (1S,5S,6S/1R,5R,6R)-1,1-dimethylethyl 1-(3,4-dichlorophenyl)-6-[(ethyloxy)methyl]-3-azabicyclo[3.1.0]hexane-3-carboxylate.

Trifluoroacetic acid (1.546 mL) was added at 0° C. to a solution of this compound in dry dichloromethane (15 mL). The mixture was then stirred at 25° C. After 1.5 h TFA (0.7 mL) was added. After 2 h the reaction mixture was concentrated in vacuo.

The crude product was purified by a SCX cartridge (10 g, eluent MeOH, then NH₃ 0.5M in MeOH) to give 557 mg the title compound.

NMR (¹, CDCl₃): δ 7.43 (s, 1H), 7.37 (d, 1H), 7.18 (d, 1H), 3.41-3.20 (m, 4H), 3.15 (s, 2H), 3.07 (m, 1H), 2.91 (d, 1H), 1.94 (m, 1H), 1.63 (m, 1H), 1.38 (m, 1H), 1.13 (t, 3H), NH not observed; MS(m/z): 286 [MH]⁺.

The enantiomers of this compound were separated by semi-preparative HPLC using a chiral column Chiralpak AD-H 25 cm×2 cm, particle size 5 micro, eluent A: n-hexane; B: ethanol 85/15 v/v, flow rate 14 mL/min, detection UV at 230 nm, obtaining the enantiomer 1 and 2 of the title compound as free base:

E12A: Enantiomer 1 (Rt.=6.5-7.5 min ) 241 mg (Example 12A)

E12B: (Enantiomer 2 (Rt.=8.0-9.5 min) 261 mg (Example 12B)

### Determination of the Absolute Configuration of 12A and 12B

Another batch of E12A (5 mg) and E12B (5 mg) was submitted for Ab Initio VCD (vibrational circular dichroism) analysis to determine the absolute configuration of these optical isomers.

Example 12A (Enantiomer 1) corresponded to (1S,5S,6S)-1-(3,4-dichlorophenyl)-6-[(ethyloxy)methyl]-3-azabicyclo[3.1.0]hexane

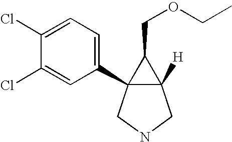

NMR (¹H, CDCl₃): δ 7.43 (s, 1H), 7.37 (d, 1H), 7.18 (d, 1H), 3.41-3.20 (m, 4H), 3.15 (s, 2H), 3.07 (m, 1H), 2.91 (d, 1H), 1.94 (m, 1H), 1.63 (m, 1H), 1.38 (m, 1H), 1.13 (t, 3H), NH not observed.

Example 12B (Enantiomer 2) corresponded to (1R,5R,6R)-1-(3,4-dichlorophenyl)-6-[(ethyloxy)methyl]-3-azabicyclo[3.1.0]hexane

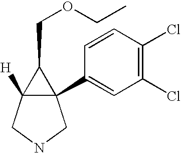

NMR (¹, CDCl₃): δ 7.43 (s, 1H), 7.37 (d, 1H), 7.18 (d, 1H), 3.41-3.20 (m, 4H), 3.15 (s, 2H), 3.07 (m, 1H), 2.91 (d, 1H), 1.94 (m, 1H), 1.63 (m, 1H), 1.38 (m, 1H), 1.13 (t, 3H), NH not observed.

### Examples 13

### (1S,5S,6S/1R,5R,6R)-1-(3,4-dichlorophenyl)-6-[(ethyloxy)methyl]-3-azabicyclo[3.1.0]hexane hydrochloride (E13)

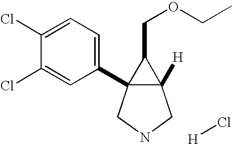

To a solution of (1S,5S,6S/1R,5R,6R)-1-(3,4-dichlorophenyl)-6-[(ethyloxy)methyl]-3-azabicyclo[3.1.0]hexane (E12, Method A, 16 mg) in DCM (0.5 mL) was added 1 equivalent of HCl (1M in Et₂O), the solvent evaporated in vacuo and the material thus obtained triturated with Et₂O to give 16 mg of the Title compound as a white slightly hygroscopic solid.

NMR (¹, CDCl₃): δ 9.24 (br. s 2H), 7.70 (d, 1H), 7.59 (d, 1H), 7.37 (dd, 1H), 3.74 (d, 1H), 3.49 (dd, 1H), 3.38 (d, 1H), 3.22 (m, 1H), 3.17 (d, 1H), 3.16 (m, 1H), 3.07 (m, 1H), 2.91 (dd, 1H), 2.20 (t, 1H), 1.64 (m, 1H), 0.94 (t, 3H); MS(m/z): 286 [MH]⁺.

### Examples 14

### (1S,5S,6S/1R,5R,6R)-1-(3,4-dichlorophenyl)-6-[(propyloxy)methyl]-3-azabicyclo[3.1.0]hexane (E14)

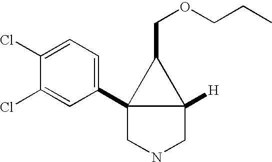

To a stirred solution of (1S,5S,6S/1R,5R,6R)-1-(3,4-dichlorophenyl)-3-azabicyclo[3.1.0]hex-6-yl]methanol (E1, 47.5 mg), (prepared following an analogous procedure to that described to obtain Example 1, method A) in dry DMF (1 mL), at 0° C., sodium hydride (7 mg, 60% in mineral oil) was added portionwise, followed by n-propyl iodide (20 μL) after 0.5 h. The reaction was stirred for 4 hours, then water (2 mL) was added and the mixture extracted with ethyl ether (5 mL×2). The organic phase was dried over anhydrous sodium sulphate and evaporated under reduced pressure to give a crude product that was treated with a 3:1 mixture of DCM and trifluoroacetic acid (6:2 mL) at room temperature for 2 h. The pH of the reaction mixture was brought to ˜9 with sodium carbonate, the mixture concentrated under reduced pressure and the residue was extracted with DCM to give 17 mg of the Title compound.

MS(m/z): 300 [MH]⁺.

### Examples 15

### (1S,5S,6S/1R,5R,6R)-1-(3,4-dichlorophenyl)-6-[(propyloxy)methyl]-3-azabicyclo[3.1.0]hexane hydrochloride (E15)

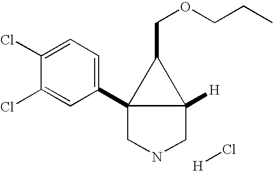

To a solution of (1S,5S,6S/1R,5R,6R)-1-(3,4-dichlorophenyl)-6-[(propyloxy)methyl]-3-azabicyclo[3.1.0]hexane (E14, 17 mg) in DCM (0.5 mL) was added 1 equivalent of HCl (1M in Et₂O), the solvent was evaporated in vacuo and the material thus obtained triturated with Et₂O to give 18 mg of the title compound as a white slightly hygroscopic solid.

NMR (¹, CDCl₃): δ 9.24 (br. s 2H), 7.72 (d, 1H), 7.60 (d, 1H), 7.39 (dd, 1H), 3.78 (d, 1H), 3.50 (dd, 1H), 3.40 (d, 1H), 3.23 (m, 1H), 3.16 (m, 2H), 2.97 (m, 1H), 2.91 (dd, 1H), 2.22 (m, 1H), 1.66 (m, 1H), 1.34 (m, 2H), 0.73 (t, 3H); MS(m/z): 300 [MH]⁺.

Example 15 (16 mg) was separated by semi-preparative HPLC using a chiral column Chiralpak AD-H, 25×0.46 cm, eluent A: n-hexane; B: ethanol+0.1% isopropylamine 85/15 v/v, flow rate 0.8 mL/min, detection UV at 235 nm, obtaining the enantiomer 1 and 2 as free base:

Example 14A: (1S,5S,6S or 1R,5R,6R)-1-(3,4-dichlorophenyl)-6-[(propyloxy)methyl]-3-azabicyclo[3.1.0]hexane (Enantiomer 1) (Rt.=5.494 min ) 4.7 mg

Example 14B: (1R,5R,6R or 1S,5S,6S)-1-(3,4-dichlorophenyl)-6-[(propyloxy)methyl]-3-azabicyclo[3.1.0]hexane (Enantiomer 2) (Rt.=6.876 min) 2.7 mg.

### Example 16

### (1S,5S,6S/1R,5R,6R)-1-(3,4-dichlorophenyl)-6-{[(2,2,2-trifluoroethyl)oxy]methyl}-3-azabicyclo[3.1.0]hexane (E16)

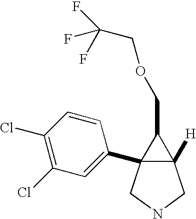

To a solution of [(1S,5S,6S/1R,5R,6R)-1-(3,4-dichlorophenyl)-3-azabicyclo[3.1.0]hex-6-yl]methanol (E1, obtained following an analogous procedure to that described to obtain Example 1, method B) (268 mg) in DCM (10 mL), at room temperature, TEA (217 μL) and Boc₂O (250 mg) were added and the reaction mixture was stirred at room temperature for 4 h. Aqueous saturated NH₄Cl solution was then added and the organic layer was separated, washed with aqueous saturated NaHCO₃ solution, dried and evaporated in vacuo. The crude product was purified by flash chromatography eluting with cyclohexane/ethyl acetate from 9/1 to 1/1 to give 300 mg of white foam.

To a solution of this material (150 mg) in DCM (4 mL), at room temperature, TEA (87 μL) and methanesulphonyl chloride (46 μL) were added and the mixture was stirred at room temperature overnight. Aqueous saturated NH₄Cl solution was added and then the organic layer was separated, washed with aqueous saturated NaCl solution, dried and evaporated in vacuo.

The crude product was dissolved in DMF (1 mL) and it was added to a mixture of 2,2,2-trifluoroethanol (61 μL) and NaH 60% (33 mg) in DMF (3 mL).

The mixture was stirred at room temperature for 2 h, heated to 60° C. for 3 h and then stirred at room temperature overnight. Aqueous saturated NH₄Cl solution was added to the reaction mixture and the aqueous phase was extracted with Et₂O. The organic layer was separated, washed with aqueous saturated NaCl solution, dried and evaporated in vacuo.

The crude product was purified by flash chromatography eluting with cyclohexane/ethyl acetate from 9/1 to 7/3 to give 120 mg as a white foam.

To a solution of this material (120 mg) in DCM (3 mL), at room temperature, was added TFA (0.5 mL) and the mixture was stirred at room temperature for 2 h. The solution was then concentrated in vacuo. The crude product was purified by SCX cartridge eluting with NH₃ 2M in MeOH to give the title compound (80 mg).

NMR (¹, CDCl₃) δ 7.41 (m, 2H), 7.16 (d, 1H), 3.67-3.51 (m, 3H), 3.35 (d, 1H), 3.27 (t, 1H), 3.16 (m, 2H), 2.92 (d, 1H), 1.67 (s, 1H), 1.41 (m, 1H). MS (m/z): 340 [MH]⁺.

Example 16 (78 mg) was separated by semi-preparative HPLC using a chiral column Chiralpak AD-H, 25×0.46 cm, eluent A: n-hexane; B: ethanol 90/10 v/v, flow rate 1 mL/min, detection UV at 230 nm, obtaining the enantiomer 1 and 2 as free base:

Example 16A: (1S,5S,6S or 1R,5R,6R)-1-(3,4-dichlorophenyl)-6-{[(2,2,2-trifluoroethyl)oxy]methyl}-3-azabicyclo[3.1.0]hexane (Enantiomer 1) (Rt.=5.996 min ) 32 mg

Example 16B: (1R,5R,6R or 1S,5S,6S)-1-(3,4-dichlorophenyl)-6-{[(2,2,2-trifluoroethyl)oxy]methyl}-3-azabicyclo[3.1.0]hexane (Enantiomer 2) (Rt.=7.406 min) 35 mg.

### Example 17

### ([(1S,5S,6S/1R,5R,6R)]-6-[(methyloxy)methyl]-1-(2-naphthalenyl)-3-azabicyclo[3.1.0]hexane (E17)

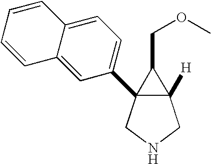

To a stirred solution of [(1S,5S,6S/1R,5R,6R or 1S,5S,6R/1R,5R,6S )-1-(2-naphthalenyl)-3-azabicyclo[3.1.0]hex-6-yl]methanol (P10, 70 mg) in DCM (3 mL) at room temperature, triethylamine (61 μL) and bis(1,1-dimethylethyl)dicarbonate (70 mg) were subsequently added. Stirring was continued for 2 h, then the reaction mixture was concentrated under vacuum and the crude product treated with dichloromethane and water. The organic phase was dried over sodium sulfate and the solvent evaporated under vacuum to give a crude product. To a stirred solution of this crude material in dry DMF (3 mL) sodium hydride (23 mg, 60% in mineral oil) was added at 0° C. and the reaction mixture stirred for 0.5 h, after which time methyl iodide (27 μL) was added and the reaction mixture was stirred at room temperature overnight. Ethyl acetate and icy water were added, the organic phase separated, washed with brine, dried over sodium sulfate and the solvent evaporated under vacuum to give a crude product. To a solution of this crude in dichloromethane (4 mL) TFA (1 ml) was added at 0° C. The reaction mixture was stirred at room temperature for 1 h. It was concentrated in vacuo and the crude product was purified by flash chromatography first using a silica column (eluting with dichloromethane/methanol/33% aqueous ammonia 95:5:0.5) and then using an amine column (eluting with ethyl acetate/cyclohexane from 2/8 to 8/2) to give 52 mg of the title compound.

NMR (¹, CDCl₃): δ 7.86-7.76 (m, 4H), 7.52-7.45 (m, 3H), 3.44 (d, 1H), 3.28-3.25 (m, 3H), 3.19 (s, 3H), 3.15-3.04 (m, 2H), 1.84 (m, 1H), 1.45 (m, 1H), NH not observed.

Example 17 (50 mg) was separated by SFC HPLC using a chiral column Chiralcel OD-H, 25×0.46 cm, eluent ethanol+0.1% isopropylamine 25%, flow rate 2 mL/min, detection UV at 230 nm, obtaining the enantiomer 1 and 2 as free base

Example 17A: ([(1S,5S,6S or 1R,5R,6R)]-6-[(methyloxy)methyl]-1-(2-naphthalenyl)-3-azabicyclo[3.1.0]hexane (Enantiomer 1) (Rt.=5.193 min ) 14 mg

Example 17B: ([(1R,5R,6R or 1S,5S,6S)]-6-[(methyloxy)methyl]-1-(2-naphthalenyl)-3-azabicyclo[3.1.0]hexane (Enantiomer 2) Rt.=12.689 min) 13 mg.

The configuration of the stereogenic centers in Example 17 was assigned on the basis of spectroscopic properties of the respective enantiomers obtained by chiral separation as above reported. The relative configuration was assessed on the basis of Roesy data.

### Example 18

### (1S,5S,6S or 1R,5R,6R)-1-(3,4-dichlorophenyl)-6-[(ethyloxy)methyl]-3-methyl-3-azabicyclo[3.1.0]hexane (E18)

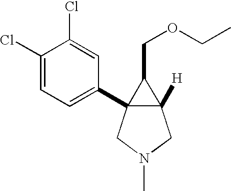

Acetic acid (0.016 mL), formaldehyde (37% solution in water, 0.061 mL) and sodium triacethoxyborohydride (28.9 mg) were added to a solution of (1S,5S,6S or 1R,5R,6R)-1-(3,4-dichlorophenyl)-6-[(ethyloxy)methyl]-3-azabicyclo[3.1.0]hexane (E12A, Enantiomer 1, 26 mg) in methanol (1 mL). The reaction mixture was stirred at 25° C. After 5 h acetic acid (0.016 mL), formaldehyde (0.061 mL) and sodium triacethoxyborohydride (28.9 mg) were added. After 6 h the reaction mixture was concentrated in vacuo, dichloromethane was added and the organic phase was washed with an aqueous saturated NaHCO₃ solution and water, dried over Na₂SO₄ and concentrated in vacuo. The crude was purified by flash chromatography eluting with dichloromethane/methanol from 100/0 to 96/4 to give 27 mg of the title compound as a colourless oil.

NMR (¹, CDCl₃): δ 7.42 (s, 1H), 7.35 (d, 1H), 7.15 (d, 1H), 3.38-3.28 (m, 2H), 3.26-3.12 (m, 3H), 3.05-2.97 (m, 1H), 2.54 (m, 1H), 2.33 (s, 3H), 2.32 (m, 1H), 1.98 (m, 1H), 1.61 (m, 1H), 1.07 (t, 3H); MS(m/z): 300 [MH]⁺.

### Example 19

### (1S,5S,6S or 1R,5R,6R)-1-(3,4-dichlorophenyl)-6-[(ethyloxy)methyl]-3-methyl-3-azabicyclo[3.1.0]hexane hydrochloride (E19)

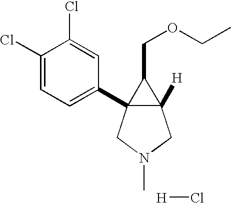

HCl (1M in diethyl ether, 93 μL) was added to a solution of (1S,5S,6S or 1R,5R,6R)-1-(3,4-dichlorophenyl)-6-[(ethyloxy)methyl]-3-methyl-3-azabicyclo[3.1.0]hexane (E18, 27 mg) in dichloromethane (4 mL). The mixture was evaporated and the residue triturated with diethyl ether to give the title compound as a white slightly hygroscopic solid (24 mg).

### Example 20

### (1R,5R,6R/1S,5S,6S)-1-(3,4-dichlorophenyl)-6-{[(1-methylethyl)oxy]methyl}-3-azabicyclo[3.1.0]hexane (E20)

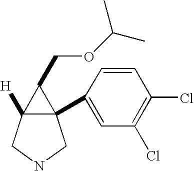

Method A:

To a solution of trifluoromethyl(1R,5R,6R/1S,5S,6S)-1-(3,4-dichlorophenyl)-6-(hydroxymethyl)-3-azabicyclo[3.1.0]hexane-3-carboxylate (P11, 290 mg) in CH₂Cl₂ (5 mL), at 0° C., TEA (0.126 mL) and methansulfonyl chloride (0.089 mL) were added. The reaction mixture was stirred at room temperature overnight and then it was quenched with NH₄Cl sat (aq) and diluted with CH₂Cl₂. The organic layer was dried and concentrated in vacuo. The crude material was purified by chromatography on silica (gradient cyclohexane/ethyl acetate from 90/10 to 70/30) to afford [(1R,5R,6R/1S,5S,6S)-1-(3,4-dichlorophenyl)-3-(trifluoroacetyl)-3-azabicyclo[3.1.0]hex-6-yl]methyl methanesulfonate as crude material (316 mg).

2-Propanol (0.113 mL) was added to a suspension of NaH (60% in mineral oil, 58.5 mg) in dry DMF (3 mL) at 0° C. After 20 min the suspension was allowed to warm to room temperature and stirred for an additional 20 min. A solution of [(1R,5R,6R/1S,5S,6S)-1-(3,4-dichlorophenyl)-3-(trifluoroacetyl)-3-azabicyclo[3.1.0]hex-6-yl]methyl methanesulfonate previously obtained (316 mg) in dry DMF (2 mL) was then added and the reaction was heated to 60° C. for 2 h. After cooling to room temperature, MeOH was added to the solution. The reaction mixture was passed through SCX cartridge eluting first with MeOH and then with NH₃ 2M in MeOH. The MeOH/NH₃ fractions were concentrated under reduced pressure and the residue was purified by flash chromatography on silica (gradient CH₂Cl₂/MeOH/NH₄OH from 99/1/0.1 to 9/1/0.1). The compound thus obtained (44 mg) was further purified by LC chromatography (Column XBridge Prep C18, Mobile phase: A: NH₄HCO₃ 10 mM aq. sol, pH=10; B: CH₃CN; gradient: 25% (B) for 1 min, 25% (B)→45 in 12.5 min, 45% (B)→100% (B) in 1.5 min, flow rate: 17 ml/min, UV range 210-350 nm). 10 mg of (1R,5R,6R/1S,5S,6S)-1-(3,4-dichlorophenyl)-6-{[(1-methylethyl)oxy]methyl}-3-azabicyclo[3.1.0]hexane were obtained.

Rt: 4.70 min, MS (m/z): 300 [MH]+.

Method B:

2-Propanol (0.064 mL) was added to a suspension of NaH (60% in mineral oil, 34 mg) in dry DMF (4 mL) at 0° C. After 30 min the suspension was allowed to warm to room temperature and a solution of 1,1-dimethylethyl(1R,5R,6R/1S,5S,6S)-1-(3,4-dichlorophenyl)-6-{[(methylsulfonyl)oxy]methyl}-3-azabicyclo[3.1.0]hexane-3-carboxylate (obtained following an analogous procedure to that described to obtain P14, 183 mg) in dry DMF (2 mL) was added. The reaction was heated to 60° C. for 6 h. After cooling to room temperature, it was quenched with NH₄Cl sat (aq) and diluted with Et₂O. The organic phase was washed with brine, dried and concentrated in vacuo. The crude product was purified by column chromatography (gradient cyclohexane/ethyl acetate from 90/10 to 80/20) to give still impure 1,1-dimethylethyl(1R,5R,6R/1S,5S,6S)-1-(3,4-dichlorophenyl)-6-{[(1-methylethyl)oxy]methyl}-3-azabicyclo[3.1.0]hexane-3-carboxylate (120 mg). It was dissolved in dry dichloromethane (3 mL) and trifluoroacetic acid (0.5 mL) was added at 0° C. After 2 h the reaction mixture was concentrated in vacuo. The crude product was purified by a SCX cartridge eluting first with MeOH and then NH₃ 2.0M in MeOH, and then by flash chromatography on silica (gradient from 100% DCM to DCM/MeOH/NH₄OH 9/1/0.1) to give 8 mg of the title compound.

NMR (¹H, CDCl₃): δ ppm 7.48 (1H, d), 7.39 (1H, d), 7.19 (1H, dd), 3.36-3.46 (2H, m), 3.24-3.35 (3H, m), 2.94-3.06 (2H, m), 1.65-1.71 (1H, m), 1.44-1.53 (1H, m), 1.08 (3H, d), 1.01 (3H, d); MS (m/z): 300 [MH]+.

The racemic mixture (E20 Method A, 10 mg) was submitted to chiral chromatography for separation into single enantiomers (Analitical conditions SFC; column Chiralcel OD-H, modifier: ethanol+0.1% isopropylamine 12%, flow rate 2.0 ml/min, pressure 100 bar, temperature 35° C., DAD 210-340 nm).

Example 20A (1R,5R,6R or 1S,5S,6S)-1-(3,4-dichlorophenyl)-6-{[(1-methylethyl)oxy]methyl}-3-azabicyclo[3.1.0]hexane: Enantiomer 1: Rt: 6.081 min, 1.3 mg.,

Example 20B (1R,5R,6R or 1S,5S,6S)-1-(3,4-dichlorophenyl)-6-{[(1-methylethyl)oxy]methyl}-3-azabicyclo[3.1.0]hexane: Enantiomer 2: Rt: 8.612 min, 1.3 mg.

### Example 21

### (1R,5R,6R/1S,5S,6S)-1-(3,4-dichlorophenyl)-6-{[(1-methylethyl)oxy]methyl}-3-azabicyclo[3.1.0]hexane hydrochloride (E21)

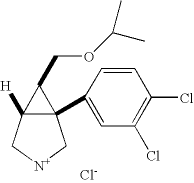

To (1R,5R,6R/1S,5S,6S)-1-(3,4-dichlorophenyl)-6-{[(1-methylethyl)oxy]methyl}-3-azabicyclo[3.1.0]hexane (E20, Method B, 6 mg) 1.0 equivalent of HCl 1M in diethyl ether was added at 25° C. The volatiles were evaporated to give the title compound (6 mg).

### Example 22

### (1R,5R,6R/1S,5S,6S)-6-[(cyclobutyloxy)methyl]-1-(3,4-dichlorophenyl)-3-azabicyclo[3.1.0]hexane (E22)

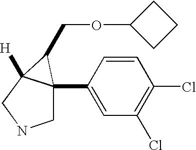

Cyclobutanol (0.026 mL) was added to a suspension of NaH (60% in mineral oil, 13.30 mg) in dry DMF (1 mL) at 0° C. After 20 min the suspension was allowed to warm at room temperature and stirred for 20 min. A solution of 1,1-dimethylethyl(1R,5R,6R/1S,5S,6S)-6-(bromomethyl)-1-(3,4-dichlorophenyl)-3-azabicyclo[3.1.0]hexane-3-carboxylate (P13, 70 mg) in dry DMF (1 mL) was then added and the reaction was stirred at room temperature for 3 h. The reaction was quenched with saturated NH4Cl (aq) solution and the aqueous phase was extracted twice with Et₂O. The combined organic phases were washed with brine, dried and concentrated in vacuo. The crude 1,1-dimethylethyl (1R,5R,6R/1S,5S,6S)-6-[(cyclobutyloxy)methyl]-1-(3,4-dichlorophenyl)-3-azabicyclo[3.1.0]hexane-3-carboxylate thus obtained (52 mg) was dissolved in CH₂Cl₂ (2 mL), at 0° C. and TFA (0.100 mL) was added. The reaction mixture was stirred at r.t. for 2 h and then the solution was concentrated in vacuo. The residue was purified by SCX cartridge eluting first with MeOH and then with NH₃ 2M in MeOH. The MeOH/ammonia fractions were concentrated in vacuo affording the title compound (E22, 35 mg).

NMR (¹, CDCl₃): δ ppm 7.42-7.49 (m, J=1.26 Hz, 1H), 7.16 (dd, J=8.15, 1.45 Hz, 1 H), 3.65-3.78 (m, 1H), 3.33 (d, J=11.49 Hz, 1H), 3.13-3.23 (m, 3H), 2.87-3.04 (m, 2H), 1.97-2.20 (m, 4H), 1.80-1.92 (m, 1H), 1.58-1.72 (m, 3H), 1.33-1.51 (m, 2H); MS (m/z): 312 [MH]+.

### Examples E22A and E22B

### (1R,5R,6R or 1S,5S,6S)-6-[(cyclobutyloxy)methyl]-1-(3,4-dichlorophenyl)-3-azabicyclo[3.1.0]hexane hydrochloride (E22A) and (1R,5R,6R or 1S,5S,6S)-6-[(cyclobutyloxy)methyl]-1-(3,4-dichlorophenyl)-3-azabicyclo[3.1.0]hexane hydrochloride (E22B)

31 mg of the racemic mixture (E22) were submitted for semi preparative chiral separation. (Chromatography conditions: column Chiralcel OD-H, mobile phase: n-Hexane/Isopropanol 90/10% v/v, flow rate 1.0 ml/min, DAD 210-340 nm).

Enantiomer 1, Rt: 7.93 min, 10 mg.

Enantiomer 2: Rt: 10.02 min, 10 mg,

To a solution of Enantiomer 1 in dichloromethane was added HCl (1 eq, 1M in Et₂O); the solvent was evaporated under vacuum to give 10 mg of the corresponding hydrochloride salt, E22A.

To a solution of Enantiomer 2 in dichloromethane was added HCl (1 eq, 1M in Et₂O); the solvent was evaporated under vacuum to give 6 mg of the corresponding hydrochloride salt, E22B.

### Example 23

### (1R,5R,6R/1S,5S,6S)-6-[(cyclopentyloxy)methyl]-1-(3,4-dichlorophenyl)-3-azabicyclo[3.1.0]hexane (E23)

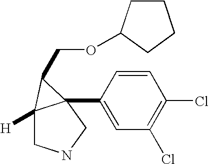

Cyclopentanol (28.6 mg) was added to a suspension of NaH (60% in mineral oil, 13.30 mg) in dry DMF (1 mL) at 0° C. After 20 min the suspension was allowed to warm at room temperature and stirred for 20 min. A solution of 1,1-dimethylethyl(1R,5R,6R/1S,5S,6S)-6-(bromomethyl)-1-(3,4-dichlorophenyl)-3-azabicyclo[3.1.0]hexane-3-carboxylate (P13, 70 mg) in dry DMF (1 mL) was added and the reaction was stirred at room temperature for 3 h and then 3 h at 60° C. Aqueous NH4Cl saturated solution was added and the aqueous phase was extracted twice with Et₂O. The combined organic phases were washed with brine, dried and concentrated in vacuo. The crude material 1,1-dimethylethyl (1R,5R,6R/1S,5S,6S)-6-[(cyclopentyloxy)methyl]-1-(3,4-dichlorophenyl)-3-azabicyclo[3.1.0]hexane-3-carboxylate thus obtained (70 mg) was dissolved in CH₂Cl₂ (2 mL), at 0° C. and TFA (0.126 mL) was added. The reaction mixture was stirred at r.t. for 2 h and then it was concentrated under reduced pressure. The residue was purified by SCX cartridge eluting first with MeOH and then with NH₃ 2M in MeOH. The MeOH/ammonia fractions were concentrated in vacuo affording the title compound (22 mg).

NMR (¹H, CDCl₃): δ ppm 7.46 (d, 1H), 7.37 (d, 1H), 7.17 (dd, 1H), 3.60-3.68 (m, 1H), 3.30-3.39 (m, 2H), 3.13-3.17 (m, 2H), 2.87-2.97 (m, 2H), 1.30-1.86 (m, 11H); MS (m/z): 326 [MH]+.

### Examples 23A and 23B

### (1R,5R,6R or 1S,5S,6S)-6-[(cyclopentyloxy)methyl]-1-(3,4-dichlorophenyl)-3-azabicyclo[3.1.0]hexane hydrochloride (E23A) and (1R,5R,6R or 1S,5S,6S)-6-[(cyclopentyloxy)methyl]-1-(3,4-dichlorophenyl)-3-azabicyclo[3.1.0]hexane hydrochloride (E23B)

20 mg of the racemic mixture (E23) were submitted for semi preparative chiral separation (chromatography conditions: column Chiralpak AD-H, mobile phase: n-Hexane/Ethanol+0.1% isopropylamine 80/20% v/v, flow rate 0.8 ml/min, DAD 210-340 nm).

Enantiomer 1 (6 mg), Rt: 5.24 min, 100% ee,

Enantiomer 2 (6 mg) Rt: 6.60 min, >99% ee,

To a solution of Enantiomer 1 in dichloromethane was added HCl (1 eq, 1M in Et₂O); the solvent was evaporated under vacuum to give 7 mg of the corresponding hydrochloride salt, E23A.

To a solution of Enantiomer 2 in dichloromethane was added HCl (1 eq, 1M in Et₂O); the solvent was evaporated under vacuum to give 7 mg of the corresponding hydrochloride salt E23B.

### Example 24

### (1R,5R,6R/1S,5S,6S)-6-[(cyclohexyloxy)methyl]-1-(3,4-dichlorophenyl)-3-azabicyclo[3.1.0]hexane trifluoroacetate (E24)

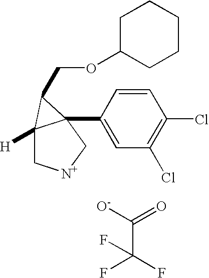

Cyclohexanol (0.040 mL) was added to a suspension of NaH (60% in mineral oil, 15.22 mg) in dry DMF (1 mL) at 0° C. After 20 min the suspension was allowed to warm at room temperature and stirred for additional 20 min. A solution of 1,1-dimethylethyl (1R,5R,6R/1S,5S,6S)-1-(3,4-dichlorophenyl)-6-{[(methylsulfonyl)oxy]methyl}-3-azabicyclo[3.1.0]hexane-3-carboxylate (P14, 80 mg) in dry DMF (1 mL) was then added and the reaction was stirred 4 h at 60° C. Aqueous NH₄Cl saturated solution was added and aqueous phase was extracted twice with Et₂O. The combined organic phases were washed with brine, dried and concentrated in vacuo. The crude material was purified by chromatography on silica gel (Gradient cyclohexane/EtOAc from 90/10 to 80/20) to give 1,1-dimethylethyl(1R,5R,6R/1S,5S,6S)-6-[(cyclohexyloxy)methyl]-1-(3,4-dichlorophenyl)-3-azabicyclo[3.1.0]hexane-3-carboxylate (24 mg). The product thus obtained (24 mg) was dissolved in CH₂Cl₂ (2 mL), at 0° C. and TFA (0.042 mL) was added. The reaction mixture was stirred at r.t. for 2 h and then it was concentrated in vacuo. The residue was purified by SCX cartridge eluting first with MeOH and then with NH₃ 2M in MeOH. The MeOH/ammonia fractions were concentrated in vacuo affording still impure (1R,5R,6R/1S,5S,6S)-6-[(cyclohexyloxy)methyl]-1-(3,4-dichlorophenyl)-3-azabicyclo[3.1.0]hexane (20 mg). The product was further purified by LC/MS-FractionLynx Autopurification System to afford 3 mg of the title compound. Preparative conditions: column: XBridge PREP C18, 100×19 mm, 5 μm; mobile phase: A: H2O+0.1% TFA; B: CH3CN, gradient: 10% (B) for 1 min, 10% to 95% (B) in 12 min, 95% (B) for 1.5 min; flow rate: 17 ml/min; UV range: 210-350 nm; ionisation: ES+.

NMR (¹H, CDCl₃): δ ppm 10.43 (br. s., 1H), 9.72 (br. s., 1H), 7.51 (d, 1H), 7.44 (d, 1H), 7.20 (dd, 1H), 3.69-3.79 (m, 1H), 3.54-3.69 (m, 2H), 3.31-3.42 (m, 2H), 2.95-3.09 (m, 2H), 1.98 (t, 1H), 1.57-1.86 (m, 5H), 1.44-1.53 (m, 1H), 1.11-1.23 (m, 5H); MS (m/z):340 [MH]+.

### Example 26

### [(1S,2S,5S,6S/1R,2R,5R,6R)-1-(3,4-dichlorophenyl)-6-[(ethyloxy)methyl]-2-methyl-3-azabicyclo[3.1.0]hexane (E26)

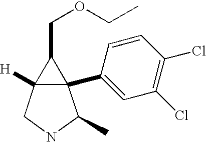

To a solution of [(1S,2R,5S,6S/1R,2S,5R,6R ) or (1S,2S,5S,6S/1R,2R,5R,6R)-3-{[2,4-bis(methyloxy)phenyl]methyl}-1-(3,4-dichlorophenyl)-6-[(ethyloxy)methyl]-2-methyl-3-azabicyclo[3.1.0]hexane (15 mg, P25) in 1,2-dichloroethane (1 mL) 1-chloroethyl chloroformate (36 μl) was added and the mixture was heated to reflux for 6 h. The reaction was cooled to RT and methanol (0.5 mL) was added. The mixture was refluxed for 2 h, cooled to RT and the residue purified by SCX cartridge eluting first with methanol and then with 2.0N NH3 in methanol. Further purification by FC on silica column (eluent DCM:MeOH=95:5) afforded the title compound (1 mg).

1H NMR (600 MHz, CHLOROFORM-d) δ ppm 7.43 (d, 1H) 7.36 (d, 1H) 7.15 (dd, 1H) 3.39 (q, 1H) 3.30-3.37 (m, 1H) 3.16-3.28 (m, 3H) 3.04 (dd, 1H) 2.98 (d, 1H) 1.44 (d, 1H) 1.31-1.35 (m, 1H) 1.19 (d, 3H) 1.12 (t, 3H).

MS (m/z): 300 [MH]⁺.

### Example 27

### [(1S,2R,5S,6S/1R,2S,5R,6R)-1-(3,4-dichlorophenyl)-6-[(ethyloxy)methyl]-2-methyl-3-azabicyclo[3.1.0]hexane (E27)

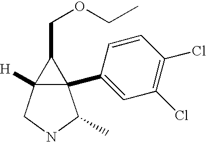

The title compound was prepared in 30 mg yield from [(1S,2R,5S,6S/1R,2S,5R,6R) or (1S,2S,5S,6S/1R,2R,5R,6R)-3-{[2,4-bis(methyloxy)phenyl]methyl}-1-(3,4-dichlorophenyl)-6-[(ethyloxy)methyl]-2-methyl-3-azabicyclo[3.1.0]hexane (63 mg, P26) following the method described in Example 26.

NMR (¹H 600 MHz, CDCl₃) δ ppm 7.44 (d, 1H) 7.35 (d, 1H) 7.14 (dd, 1H) 3.51-3.60 (m, 1H) 3.31-3.42 (m, 2H) 3.27 (d, 1H) 3.16-3.25 (m, 1H) 2.90-3.00 (m, 2H) 1.53-1.56 (m, 1H) 1.36-1.41 (m, 1H) 1.19 (d, 3H) 1.11 (t, 3H) MS (m/z): 300 [MH]⁺.

### Examples 28 and 29

### [(1S,2R,5S,6S or 1R,2S,5R,6R)-1-(3,4-dichlorophenyl)-6-[(ethyloxy)methyl]-2-methyl-3-azabicyclo[3.1.0]hexane hydrochloride (E28) and [(1S,2R,5S,6S or 1R,2S,5R,6R)-1-(3,4-dichlorophenyl)-6-[(ethyloxy)methyl]-2-methyl-3-azabicyclo[3.1.0]hexane hydrochloride (E29)

[(1S,2R,5S,6S/1R,2S,5R,6R)-1-(3,4-dichlorophenyl)-6-[(ethyloxy)methyl]-2-methyl-3-azabicyclo[3.1.0]hexane) (30 mg, E27) was submitted to semi-preparative chiral HPLC to give single enantiomers, (Conditions: chiral column chiralpak AD-H, eluent A: n-hexane; B: Ethanol, gradient isocratic 5% B, flow rate 14 mL/min, detection UV at 230 nm). Retention times given were obtained using an analytical HPLC using a chiral column chiralpak AD-H, 25×4.6 cm, eluent A: n-hexane; B: ethanol, gradient isocratic 5% B, flow rate 1 mL/min, detection UV at 230 nm.

Enantiomer 1, Rt.=8.92 min.

Enantiomer 2, Rt.=11.16 min.

To a solution of Enantiomer 1 in dichloromethane (1 mL) was added HCl (1 eq, 1M in Et₂O); the solvent was evaporated under vacuum and the material thus obtained triturated with Et₂O to give 8 mg of the corresponding hydrochloride salt as a white slightly hygroscopic solid, E28.

To a solution of Enantiomer 2 in dichloromethane (1 mL) was added HCl (1 eq, 1M in Et₂O); the solvent was evaporated under vacuum and the material thus obtained triturated with Et₂O to give 8 mg of the corresponding hydrochloride salt as a white slightly hygroscopic solid, E29.

NMR (¹H 400 MHz, DMSO-d₆) ppm 8.14 (m, 1H), 8.04 (d, 1H), 7.82 (dd, 1H), 4.51 (m, 1H), 4.15 (d, 2H), 3.70-3.62 (m, 3H), 3.55 (m, 1H), 3.36 (m, 1H), 2.64 (m, 1H), 2.19 (m, 1H), 1.75 (d, 3H), 1.39 (t, 3H); MS (m/z): 300 [MH]⁺.

### Example 30

### (1S,5S/1R,5R)-1-(3,4-dichlorophenyl)-6-[(1R/1S)-1-(methyloxy)ethyl]-3-azabicyclo[3.1.0]hexane (E30)

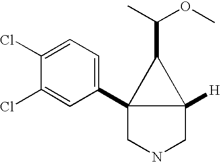

To a stirred solution of mercuric (II) acetate (0.124 g) in water (0.4 mL), at RT, THF (0.4 mL) was added followed by a solution of 1,1-dimethylethyl-(1R,5S,6S/1S,5R,6R)-1-(3,4-dichlorophenyl)-6-ethenyl-3-azabicyclo[3.1.0]hexane-3-carboxylate (P27, 0.138 g) in THF (0.4 mL). The mixture was stirred for 0.5 h then 3M NaOH (0.4 mL) was added dropwise, followed by 61 mg of a 12 wt % solution of NaBH₄ in 14M NaOH. The reaction mixture was stirred for 15 min then it was diluted with ethyl acetate. The organic phase washed with brine, dried over sodium sulphate and the solvent removed under reduce pressure to give a crude mixture containing the (1S,5S/1R,5R) 1,1-dimethylethyl(1-(3,4-dichlorophenyl)-6-[(1R/1S) 1-hydroxyethyl]-3-azabicyclo[3.1.0]hexane-3-carboxylate (MS (m/z): 316 [MH —C₄H₈]⁺).

This crude product was dissolved in THF (1.5 mL), NaH (0.031 g, 60% in oil) was added portionwise at RT and the reaction mixture was stirred for 0.5 h. After this period of time methyl iodide (0.061 mL) was added and the mixture stirred for 4 h; an additional amount of methyl iodide (0.061 mL) was added and the reaction mixture warmed to 70° C. and stirred for 4 h. The reaction mixture was then allowed to reach room temperature and stirred overnight. Saturated NH₄Cl was added, the mixture extracted with ethyl acetate, the organic phase was washed with brine, dried over sodium sulphate and evaporated obtaining a crude product that was purified by FC (eluting with cyclohexane/ethyl acetate from 1/0 to 8/2) to give 25 mg of the N-Boc intermediate. This was dissolved in 2 mL of DCM and TFA (0.1 mL) was added at RT and stirred for 1 h. The mixture was diluted with additional DCM (5 mL) and 1M NaOH was added up to basic pH. The organic phase was separated through a separator phase cartridge and the solvent was then removed under reduced pressure to give 14 mg of an oil. This product was dissolved in MeOH and purified through a SCX cartridge, eluting first with methanol and then with 2M NH₃ in methanol, obtaining 12 mg of the title compound as an oil.

1H NMR, CDCl₃: δ ppm 7.36 (d, 1H) 7.28 (d, 1H) 7.03 (dd, 1H) 3.32 (d, 1H) 3.23 (s, 3H) 3.15 (d, 1H) 3.08 (dd, 1H) 2.88 (d, 1H) 2.65-2.74 (m, 1H) 1.93-1.97 (m, 1H) 1.05 (dd, 1H) 0.98 (d, 3H); MS(m/z): 286 [MH]⁺.

### Example 31

### (1R,5R,6R/1S,5S,6S) and (1R,5R,6S/1S,5S,6R) -1-(3,4-dichlorophenyl)-6-[(ethyloxy)methyl]-6-methyl-3-azabicyclo[3.1.0]hexane (E31)

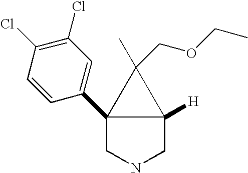

To a stirred solution of 1,1-dimethylethyl-(1R,5R,6R/1S,5S,6S)-1-(3,4-dichlorophenyl)-6-(hydroxymethyl)-6-methyl-3-azabicyclo[3.1.0]hexane-3-carboxylate and 1,1-dimethylethyl-(1R,5R,6S/1S,5S,6R)-1-(3,4-dichlorophenyl)-6-(hydroxymethyl)-6-methyl-3-azabicyclo[3.1.0]hexane-3-carboxylate (P29, 42 mg) in THF (2 mL), at room temperature, NaH (6.77 mg, 60% in oil) was added portionwise. After 20 min, ethyl iodide (0.027 mL) was added and the reaction mixture stirred at room temperature for 3 h. An additional amount of NaH (6.77 mg, 60% in oil) and ethyl iodide (0.027 mL) were added and the reaction mixture was stirred at room temperature overnight. Diethyl ether and saturated NH₄Cl were added to the reaction mixture; the organic phase was washed with brine, dried over sodium sulphate and evaporated under reduced pressure. The crude product was purified by FC (eluting with cyclohexane/ethyl acetate from 1/0 to 8/2) to give 24 mg of the corresponding ethyl derivative. This product was dissolved in DCM (0.5 mL), TFA (0.05 mL) was added and the reaction mixture was stirred for 1 h. Volatiles were removed under reduced pressure, the residue treated with DCM and aqueous saturated Na₂CO₃. The organic phase separated through a separation phase cartridge and then evaporated under reduced pressure. The crude product was purified by FC (eluting with DCM/MeOH from 1/0 to 47/3) to give 5 mg of the title compound.

¹H NMR CDCl₃: δ ppm: 7.39-7.43 (m, 1H) 7.35 (d, 1H) 7.10-7.15 (m, 1H) 3.22-3.61 (m, 4H) 2.99-3.18 (m, 4H) 1.29-1.31 (s, 3H) 1.26 (t, 1H) 1.07 (t, 3H). MS(m/z): 300 [MH]⁺.

### Example 32

### [(1R,5S,6S/1S,5R,6R)-1-(3,4-dichlorophenyl)-3-azabicyclo[3.1.0]hex-6-yl]ethanol (E32)

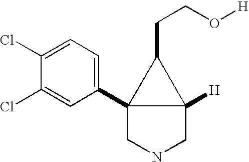

To a solution of 1,1-dimethylethyl(1R,5S,6S/1S,5R,6R)-1-(3,4-dichlorophenyl)-6-(2-hydroxyethyl)-3-azabicyclo[3.1.0]hexane-3-carboxylate (P30, 4 mg) in DCM (0.3 mL), TFA (0.050 mL) was added at room temperature. After 1 h the reaction mixture was evaporated under vacuum, the residue dissolved in methanol (0.3 mL) and 1 M NaOH (0.3 mL) was added. After 0.5 h the solvent was removed under vacuum and the residue dissolved in DCM. The organic phase was separated through a separator phase cartridge and the solvent evaporated under reduced pressure to give 1.2 mg of the title compound.

¹H NMR CDCl₃: δ ppm 7.38 (d, 1H) 7.32-7.34 (m, 1H) 7.08 (dd, 1H) 3.59-3.63 (m, 2H) 3.31 (d, 1H) 3.10-3.13 (m, 2H) 2.93 (d, 1H) 1.68 (none, 18H) 1.50-1.61 (m, 2H) 1.08-1.30 (m, 3H); MS(m/z): 272 [MH]⁺.

### Example 33

### (1R,5S,6S/1S,5R,6R)-1-(3,4-dichlorophenyl)-6-[2-(methyloxy)ethyl]-3-azabicyclo[3.1.0]hexane (E33)

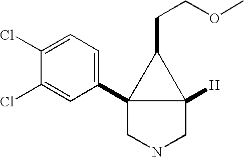

To a stirred solution of 1,1-dimethylethyl(1R,5S,6S/1S,5R,6R)-1-(3,4-dichlorophenyl)-6-(2-hydroxyethyl)-3-azabicyclo[3.1.0]hexane-3-carboxylate (P30, 73 mg) in THF (1.5 mL), at room temperature, NaH (10.98 mg, 60% in oil) was added portionwise. After 20 min, methyl iodide (0.270 mL) was added dropwise and the resulting reaction mixture was stirred overnight. Diethyl ether and aqueous saturated NH₄Cl were added, the organic phase washed with brine, dried on sodium sulphate and evaporated under reduced pressure to give the crude N-Boc intermediated. This product was dissolved in DCM (1.5 mL), TFA (0.2 mL) was added and the reaction mixture was stirred at room temperature for 1 h. After this period of time the mixture was evaporated under reduced pressure and the residue dissolved in DCM (3 mL), washed with 1M NaOH (1 mL) and separated through a phase separator cartridge. The organic phase was evaporated under reduced pressure to give 36 mg of the title compound as an oil.

¹H NMR CDCl₃ δ ppm 7.37 (dd, 1H) 7.31-7.34 (m, 1H) 7.07 (dd, 1H) 3.25-3.36 (m, 6H) 3.06-3.12 (m, 2H) 2.91 (d, 1H) 1.49-1.59 (m, 2H) 1.23-1.30 (m, 2H); MS(m/z): 286 [MH]⁺.

### Examples 34 and 35

### (1R,5S,6S or 1S,5R,6R)-1-(3,4-dichlorophenyl)-6-[2-(methyloxy)ethyl]-3-azabicyclo[3.1.0]hexane (E34) and (1R,5S,6S or 1S,5R,6R)-1-(3,4-dichlorophenyl)-6-[2-(methyloxy)ethyl]-3-azabicyclo[3.1.0]hexane (E35)

33 mg of (1R,5S,6S/1S,5R,6R)-1-(3,4-dichlorophenyl)-6-[2-(methyloxy)ethyl]-3-azabicyclo[3.1.0]hexane (E33) were submitted to semi-preparative chiral chromatography to give 7.4 mg of the Enantiomer 1 (E34) (Rt 18.82 min) and 8.4 mg of the Enantiomer 2 (E35) (Rt 22.04 min).

Chromatographic conditions:

Column: Chiralcel OD-H (25×0.46 cm)

Mobile phase: n-Hexane/2-Propanol 95/5 % v/v

Flow rate: 1.0 ml/min

DAD: 210-340 nm

CD: 230 nm

### Examples 34A and 35A

### (1R,5S,6S or 1S,5R,6R)-1-(3,4-dichlorophenyl)-6-[2-(methyloxy)ethyl]-3-azabicyclo[3.1.0]hexane hydrochloride (E34A) and (1R,5S,6S or 1S,5R,6R)-1-(3,4-dichlorophenyl)-6-[2-(methyloxy)ethyl]-3-azabicyclo[3.1.0]hexane hydrochloride (E35A)

Enantiomer 1 was dissolved in DCM (0.3 mL) and 1N HCl in ether (26 μL) was added. The solvent was evaporated under reduced pressure to give the corresponding hydrochloridric salt (8.3 mg) as a pale yellow solid (E34A).

1H NMR (500 MHz, DMSO-d₆) δ ppm 9.72 (br. s., 1H) 9.21 (br. s., 1H) 7.69 (d, 1H) 7.61 (d, 1H) 7.36 (dd, 1H) 3.66-3.86 (m, 1H) 3.40-3.54 (m, 1H) 3.30-3.42 (m, 1H) 3.12 (s, 3H) 3.08-3.26 (m, 3H) 2.13-2.24 (m, 1H) 1.37-1.48 (m, 1H) 1.27-1.39 (m, 1H) 1.02-1.13 (m, 1H). MS(m/z): 286 [MH]⁺.

Enantiomer 2 was dissolved in DCM (0.3 mL) and 1N HCl in ether (30 μL) was added. The solvent was evaporated under reduced pressure to give the corresponding hydrochloridric salt (9.4 mg) as a white solid (E35A).

### Example 36

### (1R,5S,6S/1S,5R,6R)-1-(3,4-dichlorophenyl)-6-[2-(ethyloxy)ethyl]-3-azabicyclo[3.1.0]hexane (E36)

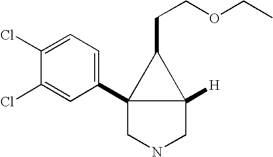

To a stirred solution of 1,1-dimethylethyl(1R,5S,6S/1S,5R,6R)-1-(3,4-dichlorophenyl)-6-(2-hydroxyethyl)-3-azabicyclo[3.1.0]hexane-3-carboxylate (obtained following an analogous procedure to that described to obtain P30, 80 mg) in THF (1.5 mL), at room temperature, NaH (15.47 mg, 60% in oil) was added portionwise. After 20 mins, ethyl iodide (0.035 mL) was added dropwise and the resulting reaction mixture was stirred overnight. Diethyl ether and a saturated solution of ammonium chloride were added. The organic phase was washed with brine, dried over sodium sulphate and evaporated under reduced pressure to give the crude N-Boc intermediate. This product was dissolved in DCM (1.5 mL), TFA (0.1 mL) was added and the reaction mixture was stirred at room temperature for 1 h. The mixture was then concentrated under reduced pressure and the residue was dissolved in DCM (3 mL), washed with 1M NaOH (1 mL) and dried through a phase separator cartridge. The organic phase was evaporated under reduced pressure to give 5.6 mg of the title compound as an oil.

¹H NMR CDCl₃ δ ppm 7.37 (d, 1H) 7.32 (d, 1H) 7.04-7.10 (m, 1H) 3.39-3.47 (m, 2H) 3.28-3.39 (m, 3H) 3.10-3.15 (m, 2H) 2.93 (d, 1H) 1.55-1.60 (m, 2H) 1.15-1.21 (m, 4H) 0.89-0.94 (m, 1H); MS(m/z): 300 [MH]⁺.

Hydrochloride salts of Examples 4A, 4B, 8A, 8B, 14A, 14B, 16A, 16B, 17A and 17B were prepared using a similar procedure as set out earlier in Example 3 (E3); amounts obtained for each compounds are summarized in the table below:

| | | | Starting | | |
|---|---|---|---|---|---|
| | | | material | Product | Analytical |
| Ex nr | SM | Chemical Name | (mg) | (mg) | Data* |
| 4A.1 | 4A | (1S,5S,6S or 1R,5R,6R)-1-(3,4- | 30 | 30 | Rt = 7.19 min |
| | | dichlorophenyl)-6-[(methyloxy)methyl]-3- | | | |
| | | azabicyclo[3.1.0]hexane hydrochloride | | | |
| | | (Enantiomer 1) | | | |
| 4B.1 | 4B | (1R,5R,6R or 1S,5S,6S)-1-(3,4- | 30 | 30 | Rt = 8.54 min |
| | | dichlorophenyl)-6-[(methyloxy)methyl]-3- | | | |
| | | azabicyclo[3.1.0]hexane hydrochloride | | | |
| | | (Enantiomer 2) | | | |
| 8A.1 | 8A | (1S,5S,6S or 1R,5R,6R)-6- | 16 | 16 | MS (m/z): |
| | | {[(cyclopropylmethyl)oxy]methyl}-1-(3,4- | | | 312 [MH]⁺ |
| | | dichlorophenyl)-3-azabicyclo[3.1.0]hydrochloride | | | |
| | | (Enantiomer 1) | | | |
| 8B.1 | 8B | (1R,5R,6R or 1S,5S,6S)-6- | 16 | 16 | MS (m/z): |
| | | {[(cyclopropylmethyl)oxy]methyl}-1-(3,4- | | | 312 [MH]⁺ |
| | | dichlorophenyl)-3-azabicyclo[3.1.0]hydrochloride | | | |
| | | (Enantiomer 2) | | | |
| 14A.1 | 14A | (1S,5S,6S or 1R,5R,6R)-1-(3,4- | 4.7 | 5.3 | |
| | | dichlorophenyl)-6-[(propyloxy)methyl]-3- | | | |
| | | azabicyclo[3.1.0]hexane hydrochloride | | | |
| | | (Enantiomer 1) | | | |
| 14B.1 | 14B | (1R,5R,6R or 1S,5S,6S)-1-(3,4- | 2.7 | 3.0 | |
| | | dichlorophenyl)-6-[(propyloxy)methyl]-3- | | | |
| | | azabicyclo[3.1.0]hexane hydrochloride | | | |
| | | (Enantiomer 2) | | | |
| 16A.1 | 16A | (1S,5S,6S or 1R,5R,6R)-1-(3,4- | 32 | 33 | NMR (¹H 400 MHz, |
| | | dichlorophenyl)-6-{[(2,2,2- | | | DMSO-d₆) δ |
| | | trifluoroethyl)oxy]methyl}-3- | | | ppm 7.72 (1H, d), |
| | | azabicyclo[3.1.0]hexane hydrochloride | | | 7.61 (1H, d), |
| | | (Enantiomer 1) | | | 7.39 (1H, dd), |
| | | | | | 3.72-3.97 (3H, m), |
| | | | | | 3.53 (1H, d), |
| | | | | | 3.35-3.47 (2H, m), |
| | | | | | 3.18-3.27 (2H, m), |
| | | | | | 2.33 (1H, t), |
| | | | | | 1.68-1.75 (1H, m); |
| 16B.1 | 16B | (1R,5R,6R or 1S,5S,6S)-1-(3,4- | 35 | 36 | NMR (¹H 400 MHz, |
| | | dichlorophenyl)-6-{[(2,2,2- | | | DMSO-d₆) δ |
| | | trifluoroethyl)oxy]methyl}-3- | | | ppm 7.72 (1H, d), |
| | | azabicyclo[3.1.0]hexane hydrochloride | | | 7.61 (1H, d), |
| | | (Enantiomer 2) | | | 7.39 (1H, dd), |
| | | | | | 3.72-3.97 (3H, m), |
| | | | | | 3.53 (1H, d), |
| | | | | | 3.35-3.47 (2H, m), |
| | | | | | 3.18-3.27 (2H, m), |
| | | | | | 2.33 (1H, t), |
| | | | | | 1.68-1.75 (1H, m); |
| 17A.1 | 17A | ([(1S,5S,6S or 1R,5R,6R)]-6- | 14 | 13 | MS (m/z): |
| | | [(methyloxy)methyl]-1-(2-naphthalenyl)-3- | | | 254 [MH]⁺ |
| | | azabicyclo[3.1.0]hexane hydrochloride | | | |
| | | (Enantiomer 1) | | | |
| 17B.1 | 17B | ([(1R,5R,6R or 1S,5S,6S)]-6- | 13 | 13 | MS (m/z): |
| | | [(methyloxy)methyl]-1-(2-naphthalenyl)-3- | | | 254 [MH]⁺ |
| | | azabicyclo[3.1.0]hexane hydrochloride | | | |
| | | (Enantiomer 2). | | | |

*HPLC conditions providing Rt are those described for obtaining the two enantiomers of the compound from the corresponding racemate.

### Example 37

### (1S,5S,6S/1R,5R,6R)-1-(3,4-dichlorophenyl)-6-{[(4-fluorophenyl)oxy]methyl}-3-azabicyclo[3.1.0]hexane (E37)

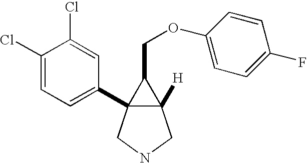

To a solution of 1,1-dimethylethyl(1S,5S,6S/1R,5R,6R)-1-(3,4-dichlorophenyl)-6-{[(4-fluorophenyl)oxy]methyl}-3-azabicyclo[3.1.0]hexane-3-carboxylate (P15, 123 mg) in dry dichloromethane (2 mL) trifluoroacetic acid (0.209 mL) was added at room temperature. After 50 min the reaction mixture was concentrated in vacuo. The crude product was purified by a SCX cartridge (2 g, eluting first with MeOH 7CV, then NH₃ 0.5M in MeOH 7CV), and then by flash chromatography (Biotage Si 25S column, eluant A: dichloromethane, B: dichloromethane/methanol 9/1, isocratic 5% B 1CV, gradient from 5% to 60% B in 10CV, isocratic 60% B 1CV, from 60% to 80% B in 3CV) to give 73 mg of the title compound as a colourless oil.

NMR (¹, CDCl₃): δ ppm 7.43 (s, 1H), 7.36 (d, 1H), 7.18 (d, 1H), 6.92 (m, 2H), 6.70 (m, 2H), 3.82 (m, 1H), 3.60 (m, 1H), 3.36 (d, 1H), 3.19 (s, 2H), 2.95 (d, 1H), 2.15 (bs, 1H) 1.75 (m, 1H), 1.58 (m, 1H). MS (m/z): 352 [MH]+.

### Example 38

### (1S,5S,6S/1R,5R,6R)-1-(3,4-dichlorophenyl)-6-{[(4-fluorophenyl)oxy]methyl}-3-azabicyclo[3.1.0]hexane hydrochloride (E38)

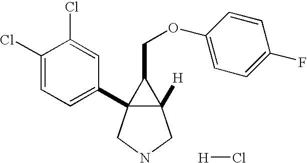

To a solution of (1S,5S,6S/1R,5R,6R)-1-(3,4-dichlorophenyl)-6-{[(4-fluorophenyl)oxy]methyl}-3-azabicyclo[3.1.0]hexane (E37, 73 mg) in dichloromethane (4 mL) HCl 1M in diethyl ether (0.20 mL) was added at 25° C. The mixture was evaporated and the residue triturated with diethyl ether to give the title compound as white solid (77 mg).

NMR (¹, DMSO): δ ppm 9.25 (bs, 2H), 7.73 (d, 1H), 7.58 (d, 1H), 7.43 (dd, 1H), 7.04 (m, 2H), 6.77 (m, 2H), 3.78-3.86 (m, 2H), 3.58-3.50 (m, 2H), 3.47-3.43 (m, 1H), 3.25 (m, 1H), 2.40 (m, 1H), 1.90 (m, 1H). MS (m/z): 352 [MH]+.

### Example 39

### {[(1S,5S,6S/1R,5R,6R)-1-(3,4-dichlorophenyl)-3-azabicyclo[3.1.0]hex-6-yl]methyl}dimethylamine (E39)

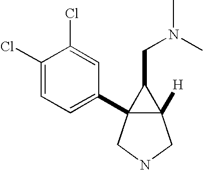

To a solution of 1,1-dimethylethyl(1R,5S,6S/1R,5R,6R)-1-(3,4-dichlorophenyl)-6-[(dimethylamino)methyl]-3-azabicyclo[3.1.0]hexane-3-carboxylate (P17, 31 mg) in dry dichloromethane (1 mL), trifluoroacetic acid (0.062 mL) was added at room temperature. After 1 h the reaction mixture was concentrated in vacuo and the residue was purified by a SCX cartridge (1 g, MeOH 10CV then NH₃ 0.5M in MeOH 10CV), to give 22 mg of the title compound as a pale yellow oil.

NMR (¹, CDCl₃): δ ppm 7.40-7.32 (m, 2H), 7.09 (m, 1H), 3.30 (d, 1H), 3.15 (s, 2H), 2.96 (d, 1H), 2.40 (m, 1H), 2.20 (s, 6H), 2.19 (bs, 1H), 1.75-1.62 (m, 2H), 1.20 (m, 1H). MS (m/z): 285 [MH]+.

### Example 40

### (1S,5S,6S/1R,5R,6R)-1-(3,4-dichlorophenyl)-6-[(methylthio)methyl]-3-azabicyclo[3.1.0]hexane (E40)

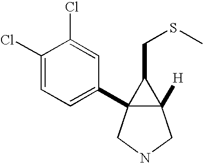

To a solution of 1,1-dimethylethyl(1S,5S,6S/1R,5R,6R)-1-(3,4-dichlorophenyl)-6-[(methylthio)methyl]-3-azabicyclo[3.1.0]hexane-3-carboxylate (P4, 16 mg) in dry dichloromethane (1 mL), trifluoroacetic acid (0.032 mL) was added at room temperature. After 30 min the reaction mixture was concentrated in vacuo and the residue was purified by a SCX cartridge (1 g, MeOH 10CV then NH₃ 0.5M in MeOH 10CV) to give 11 mg of the title compound.

NMR (¹, CDCl₃): δ ppm 7.42-7.32 (m, 2H), 7.11 (m, 1H), 3.32 (d, 1H), 3.17 (s, 2H), 2.95 (d, 1H), 2.40 (m, 1H), 2.20-2.10 (m, 1H), 2.10 (s, 3H), 2.06 (bs, 1H), 1.68 (m, 1H), 1.32 (m, 1H). MS (m/z): 288 [MH]+.

The enantiomers of the title compound were separated by chiral HPLC by using a chiral column Chiralcel OJ-H (25×0.46 cm), eluent A: n-hexane; B: 2-propanol+0.1% isopropylamine, isocratic 4% B, 10% B from 27 min, flow rate 1 mL/min, detection UV at 210-340 nm, CD 230 nm. Retention times given were obtained using an analytical HPLC using a chiral column Chiralcel OJ-H (25×0.46 cm), eluent A: n-hexane; B: 2-propanol+0.1% isopropylamine, isocratic 4% B, flow rate 1 mL/min, detection UV at 210-340 nm, CD 230 nm.

Example 41: (1S,5S,6S or 1R,5R,6R)-1-(3,4-dichlorophenyl)-6-[(methylthio)methyl]-3-azabicyclo[3.1.0]hexane (E41) (Enantiomer 1) was recovered as a colourless oil, Rt.=17.18 min (3.7 mg).

NMR (¹, CDCl₃): δ ppm 7.42-7.32 (m, 2H), 7.11 (m, 1H), 3.32 (d, 1H), 3.17 (s, 2H), 2.95 (d, 1H), 2.40 (m, 1H), 2.20-2.10 (m, 1H), 2.10 (s, 3H), 1.68 (m, 1H), 1.32 (m, 1H), NH not observed.

Example 42: (1R,5R,6R or 1S,5S,6S)-1-(3,4-dichlorophenyl)-6-[(methylthio)methyl]-3-azabicyclo[3.1.0]hexane (E42) (Enantiomer 2) was recovered as a colourless oil, Rt.=21.46 min (3.8 mg).

NMR (¹, CDCl₃): δ ppm 7.42-7.32 (m, 2H), 7.11 (m, 1H), 3.32 (d, 1H), 3.17 (s, 2H), 2.95 (d, 1H), 2.40 (m, 1H), 2.20-2.10 (m, 1H), 2.10 (s, 3H), 1.68 (m, 1H), 1.32 (m, 1H), NH not observed.

### Example 43

### (1S,5S,6S or 1R,5R,6R)-1-(3,4-dichlorophenyl)-6-[(methylthio)methyl]-3-azabicyclo[3.1.0]hexane hydrochloride (E43)

HCl 1M in diethyl ether (0.013 mL) was added at 25° C. to a solution of (1S,5S,6S or 1R,5R,6R)-1-(3,4-dichlorophenyl)-6-[(methylthio)methyl]-3-azabicyclo[3.1.0]hexane (E41, 3.7 mg) in dichloromethane (2 mL). The mixture was evaporated and the residue triturated with diethyl ether (2×) to give the title compound as a white solid (4 mg).

MS (m/z): 288 [MH]+.

### Example 44

### (1R,5R,6R or 1S,5S,6S)-1-(3,4-dichlorophenyl)-6-[(methylthio)methyl]-3-azabicyclo[3.1.0]hexane hydrochloride (E44)

HCl 1M in diethyl ether (0.013 mL) was added at 25° C. to a solution of (1R,5R,6R or 1S,5,6S)-1-(3,4-dichlorophenyl)-6-[(methylthio)methyl]-3-azabicyclo[3.1.0]hexane hydrochloride (E42, 3.8 mg) in dichloromethane (2 mL). The mixture was evaporated and the residue triturated with diethyl ether (2×) to give the title compound as a white solid (4 mg).

MS (m/z): 288 [MH]+.

1. A compound of formula (I)′ or a pharmaceutically acceptable salt thereof: 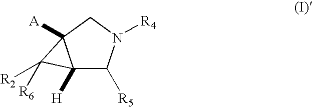
wherein
A is 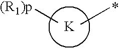
and
K is a mono or bicyclic aryl group;
R₁ is selected from the group consisting of: halogen, C₁₋₄alkyl and C₁₋₄alkoxy, and such R₁ may assume different meanings on the basis of p value;
p is an integer from 0 to 5;
R₂ is a group P, wherein P is 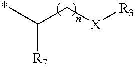
and
R₃ is hydrogen, C₁₋₄alkyl, C₃-₆cycloalkyl, C₃-₆cycloalkylC₁₋₃alkyl, haloC₁₋₂alkyl or an optionally substituted phenyl group;
X is oxygen, —NR₈— or sulphur;
n is 0 or 1;
R₇ is hydrogen or methyl;
R₈ is hydrogen or C₁₋₄alkyl;
R₄ is hydrogen or methyl;
R₅ is hydrogen or C₁₋₄alkyl; and
R₆ is hydrogen or C₁₋₄alkyl.

2-10. (canceled)

11. The compound of claim 1, wherein K is a phenyl group, p is 2 and R₁ is chloro.

12. The compound of claim 1, wherein the compound of formula (I)′ is selected from:
[(1S,5S,6R/1R,5R,6S)-1-(3,4-dichlorophenyl)-3-azabicyclo[3.1.0]hex-6-yl]methanol;
(1S,5S,6S/1R,5R,6R)-1-(3,4-dichlorophenyl)-6-[(methyloxy)methyl]-3-azabicyclo[3.1.0]hexane;
(1S,5S,6S/1R,5R,6R)-1-(3,4-dichlorophenyl)-6-[(methyloxy)methyl]-3-azabicyclo[3.1.0]hexane;
(1S,5S,6R/1R,5R,6S)-1-(3,4-dichlorophenyl)-6-[(methyloxy)methyl]-3-azabicyclo[3.1.0]hexane;
(1S,5S,6S/1R,5R,6R)-6-{[(cyclopropylmethyl)oxy]methyl}1-(3,4-dichlorophenyl)-3-azabicyclo[3.1.0]hexane;
(1S,5S,6S/1R,5R,6R)-6-{[(cyclopropylmethyl)oxy]methyl}1-(3,4-dichlorophenyl)-3-azabicyclo[3.1.0]hexane;
(1S,5S,6R/1R,5R,6S)-6-{[(cyclopropylmethyl)oxy]methyl}1-(3,4-dichlorophenyl)-3-azabicyclo[3.1.0]hexane;
(1S,5S,6S)-1-(3,4-dichlorophenyl)-6-[(ethyloxy)methyl]-3-azabicyclo[3.1.0]hexane;
(1R,5R,6R)-1-(3,4-dichlorophenyl)-6-[(ethyloxy)methyl]-3-azabicyclo[3.1.0]hexane;
(1S,5S,6S/1R,5R,6R)-1-(3,4-dichlorophenyl)-6-[(ethyloxy)methyl]-3-azabicyclo[3.1.0]hexane;
(1S,5S,6S/1R,5R,6R)-1-(3,4-dichlorophenyl)-6-[(propyloxy)methyl]-3-azabicyclo[3.1.0]hexane;
(1S,5S,6S/1R,5R,6R)-1-(3,4-dichlorophenyl)-6-{[(2,2,2-trifluoroethyl)oxy]methyl}3-azabicyclo[3.1.0]hexane;
([(1S,5S,6S/1R,5R,6R)]-6-[(methyloxy)methyl]-1-(2-naphthalenyl)-3-azabicyclo[3.1.0]hexane;
(1S,5S,6S or 1R,5R,6R)-1-(3,4-dichlorophenyl)-6-[(ethyloxy)methyl]-3-methyl-3-azabicyclo[3.1.0]hexane;
(1R,5R,6R/1S,5S,6S)-1-(3,4-dichlorophenyl)-6-{[(l -methylethyl)oxy]methyl}-3-azabicyclo[3.1.0]hexane;
(1R,5R,6R or 1S,5S,6S)-1-(3,4-dichlorophenyl)-6-{[(1-methylethyl)oxy]methyl}-3-azabicyclo[3.1.0]hexane;
(1R,5R,6R or 1S,5S,6S)-1-(3,4-dichlorophenyl)-6-{[(1-methylethyl)oxy]methyl}-3-azabicyclo[3.1.0]hexane;
(1R,5R,6R/1S,5S,6S)-1-(3,4-dichlorophenyl)-6-{[(1-methylethyl)oxy]methyl}-3-azabicyclo[3.1.0]hexane;
(1R,5R,6R/1S,5S,6S)-6-[(cyclobutyloxy)methyl]-1-(3,4-dichlorophenyl)-3-azabicyclo[3.1.0]hexane;
(1R,5R,6R or 1S,5S,6S)-6-[(cyclobutyloxy)methyl]-1-(3,4-dichlorophenyl)-3-azabicyclo[3.1.0]hexane;
(1R,5R,6R or 1S,5S,6S)-6-[(cyclobutyloxy)methyl]-1-(3,4-dichlorophenyl)-3-azabicyclo[3.1.0]hexane;
(1R,5R,6R/1S,5S,6S)-6-[(cyclopentyloxy)methyl]-1-(3,4-dichlorophenyl)-3-azabicyclo[3.1.0]hexane;
(1R,5R,6R or 1S,5S,6S)-6-[(cyclopentyloxy)methyl]-1-(3,4-dichlorophenyl)-3-azabicyclo[3.1.0]hexane;
(1R,5R,6R or 1S,5S,6S)-6-[(cyclopentyloxy)methyl]-1-(3,4-dichlorophenyl)-3-azabicyclo[3.1.0]hexane;
(1R,5R,6R/1S,5S,6S)-6-[(cyclohexyloxy)methyl]-1-(3,4-dichlorophenyl)-3-azabicyclo[3.1.0]hexane trifluoroacetate;
(1S,5R,6R/1R,5S,6S)-1-(3,4-dichlorophenyl)-6-propyl-3-azabicyclo[3.1.0]hexane trifluoroacetate;
[(1S,2S,5S,6S/1R,2R,5R,6R )-1-(3,4-dichlorophenyl)-6-[(ethyloxy)methyl]-2-methyl-3-azabicyclo[3.1.0]hexane;
[(1S,2R,5S,6S/1R,2S,5R,6R)-1-(3,4-dichlorophenyl)-6-[(ethyloxy)methyl]-2-methyl-3-azabicyclo[3.1.0]hexane;
[(1S,2R,5S,6S or 1 R,2S,5R,6R)-1-(3,4-dichlorophenyl)-6-[(ethyloxy)methyl]-2-methyl-3-azabicyclo[3.1.0]hexane;
[(1S,2R,5S,6S or 1R,2S,5R,6R)-1-(3,4-dichlorophenyl)-6-[(ethyloxy)methyl]-2-methyl-3-azabicyclo[3.1.0]hexane;
(1S,5S/1R,5R)-1-(3,4-dichlorophenyl)-6-[(1R/1S)-1-(methyloxy)ethyl]-3-azabicyclo[3.1.0]hexane;
(1R,5R,6R/1S,5S,6S) and (1R,5R,6S/1S,5S,6R)-1-(3,4-dichlorophenyl)-6-[(ethyloxy)methyl]-6-methyl-3-azabicyclo[3.1.0]hexane;
[(1R,5S,6S/1S,5R,6R)-1-(3,4-dichlorophenyl)-3-azabicyclo[3.1.0]hex-6-yl]ethanol;
(1R,5S,6S/1S,5R,6R)-1-(3,4-dichlorophenyl)-6-[2-(methyloxy)ethyl]-3-azabicyclo[3.1.0]hexane;
(1R,5S,6S or 1S,5R,6R)-1-(3,4-dichlorophenyl)-6-[2-(methyloxy)ethyl]-3-azabicyclo[3.1.0]hexane;
(1R,5S,6S or 1S,5R,6R)-1-(3,4-dichlorophenyl)-6-[2-(methyloxy)ethyl]-3-azabicyclo[3.1.0]hexane;
(1R,5S,6S/1S,5R,6R)-1-(3,4-dichlorophenyl)-6-[2-(ethyloxy)ethyl]-3-azabicyclo[3.1.0]hexane(1S,5S,6S or 1R,5R,6R)-1-(3,4-dichlorophenyl)-6-[(methyloxy)methyl]-3-azabicyclo[3.1.0]hexane;
(1R,5R,6R or 1S,5S,6S)-1-(3,4-dichlorophenyl)-6-[(methyloxy)methyl]-3-azabicyclo[3.1.0]hexane;
(1S,5S,6S or 1R,5R,6R)-6-{[(cyclopropylmethyl)oxy]methyl}-1-(3,4-dichlorophenyl)-3-azabicyclo[3.1.0];
(1R,5R,6R or 1S,5S,6S)-6-{[(cyclopropylmethyl)oxy]methyl}-1-(3,4-dichlorophenyl)-3-azabicyclo[3.1.0]; ([(1S,5S,6S or 1R,5R,6R)]-6-[(methyloxy)methyl]-1-(2-naphthalenyl)-3-azabicyclo[3.1.0]hexane;
([(1R,5R,6R or 1S,5S,6S)]-6-[(methyloxy)methyl]-1-(2-naphthalenyl)-3-azabicyclo[3.1.0]hexane;
(1S,5S,6S/1R,5R,6R)-1-(3,4-dichlorophenyl)-6-{[(4-fluorophenyl)oxy]methyl}-3-azabicyclo[3.1.0]hexane;
(1S,5S,6S/1R,5R,6R)-1-(3,4-dichlorophenyl)-6-{[(4-fluorophenyl)oxy]methyl}-3-azabicyclo[3.1.0]hexane;
{[(1S,5S,6S/1R,5R,6R)-1-(3,4-dichlorophenyl)-3-azabicyclo[3.1.0]hex-6-yl]methyl}dimethylamine;
(1S,5S,6S/1R,5R,6R)-1-(3,4-dichlorophenyl)-6-[(methylthio)methyl]-3-azabicyclo[3.1.0]hexane;
(1S,5S,6S or 1R,5R,6R)-1-(3,4-dichlorophenyl)-6-[(methylthio)methyl]-3-azabicyclo[3.1.0]hexane;
(1R,5R,6R or 1S,5S,6S)-1-(3,4-dichlorophenyl)-6-[(methylthio)methyl]-3-azabicyclo[3.1.0]hexane;
(1S,5S,6S or 1R,5R,6R)-1-(3,4-dichlorophenyl)-6-[(methylthio)methyl]-3-azabicyclo[3.1.0]hexane; and
(1R,5R,6R or 1S,5S,6S)-1-(3,4-dichlorophenyl)-6-[(methylthio)methyl]-3-azabicyclo[3.1.0]hexane.

13. A method of treating a condition for which inhibition of serotonin (5-HT), dopamine (DA) and norepinephrine (NE), is beneficial, which comprises administering to a mammal in need thereof an effective amount of the compound of claim 1.

14. The method as claimed in claim 13, wherein the condition to be treated is depression.

15. The method as claimed in claim 13, wherein the condition to be treated is an eating disorder.

16. A pharmaceutical composition comprising the compound of claim 1 and a pharmaceutically acceptable carrier.

17. A method of treating a condition for which inhibition of serotonin (5-HT), dopamine (DA) and norepinephrine (NE), is beneficial, which comprises administering to a mammal in need thereof an effective amount of the compound of claim 11.

18. The method as claimed in claim 17, wherein the condition to be treated is depression.

19. The method as claimed in claim 17, wherein the condition to be treated is an eating disorder.

20. A pharmaceutical composition comprising the compound of claim 11 and a pharmaceutically acceptable carrier.

21. A method of treating a condition for which inhibition of serotonin (5-HT), dopamine (DA) and norepinephrine (NE), is beneficial, which comprises administering to a mammal in need thereof an effective amount of the compound of claim 12.

22. The method as claimed in claim 21, wherein the condition to be treated is depression.

23. The method as claimed in claim 21, wherein the condition to be treated is an eating disorder.

24. A pharmaceutical composition comprising the compound of claim 12 and a pharmaceutically acceptable carrier.
